(19) United States
(12) Patent Application Publication
(10) Pub. No.: US 2023/0076395 A1
(43) Pub. Date: Mar. 9, 2023

(54) CELL-TO-CELL DELIVERY OF RNA CIRCUITS

(71) Applicant: California Institute of Technology, Pasadena, CA (US)

(72) Inventors: Richard F. Horns, Pasadena, CA (US); Michael B. Elowitz, Pasadena, CA (US); Joe A. Martinez, Pasadena, CA (US)

(21) Appl. No.: 17/820,232

(22) Filed: Aug. 16, 2022

Related U.S. Application Data
(60) Provisional application No. 63/234,087, filed on Aug. 17, 2021.
(60) Provisional application No. 63/234,085, filed on Aug. 17, 2021.

(51) Int. Cl. C12N 15/88; C12N 15/62; C12N 15/86; C07K 14/005

(52) U.S. Cl. CPC C12N 15/88; C12N 15/625; C12N 15/86; C07K 14/005; C12N 2740/16022; C12N 2740/16042; C07K 2319/85; C12N 2740/13022; C12N 2740/13042

(57) ABSTRACT

Disclosed herein include methods, compositions, and kits suitable for use in the delivery of polyribonucleotides and circuits. There are provided, in some embodiments, RNA exporter proteins comprising an RNA-binding domain, a membrane-binding domain, and an interaction domain capable of nucleating self-assembly. Disclosed herein include polynucleotides encoding cargo RNA molecule(s). In some embodiments, a plurality of RNA exporter proteins are capable of self-assembling into lipid-enveloped nanoparticles (LNs) secreted from a sender cell in which the RNA exporter proteins are expressed, thereby generating a population of LNs comprising a fusogen and exported cargo RNA molecule(s).

### RELATED APPLICATIONS

The present application claims priority to U.S. Provisional Application No. 63/234,087, filed Aug. 17, 2021; and U.S. Provisional Application No. 63/234,085, filed Aug. 17, 2021. The entire contents of these applications are hereby expressly incorporated by reference in their entireties.

### STATEMENT REGARDING FEDERALLY SPONSORED R&D

This invention was made with government support under Grant No. 2021552 awarded by the National Science Foundation. The government has certain rights in the invention.

### REFERENCE TO SEQUENCE LISTING

The present application is being filed along with a Sequence Listing in electronic format. The Sequence Listing is provided as a file entitled Sequence_Listing_30KJ-302451-US, created Aug. 12, 2022, which is 36.0 kilobytes in size. The information in the electronic format of the Sequence Listing is incorporated herein by reference in its entirety.

### BACKGROUND

### Field

The present disclosure relates generally to the fields of synthetic biology, cellular engineering, and polyribonucleotide delivery.

### Description of the Related Art

The ability to deploy complex genetic programs in cells within the human body could revolutionize biomedicine. For example, circuits that selectively activate cell killing or immune recruitment to tumor cells could improve the efficacy and safety of cancer therapies. Circuits that manipulate cell states, types, and behaviors, such as reprogramming cells from one type to another, in their native context within tissue (in situ) could enable new regenerative medicines. However, current gene and cell therapies are each fundamentally limited with respect to delivery. Gene therapies typically rely on viral capsids or lipid nanoparticles to deliver DNA or mRNA to cells. These methods are limited in the range and specificity of cell types they can access and the amount of genetic cargo (payload) they can deliver. Cell therapies are based on engineering of patient specific or allogeneic cells that are then introduced into a patient, but cannot deliver complex genetic programs to endogenous, non-engineered cells. There is a need for compositions, methods, systems, and kits for the delivery of polyribonucleotides and circuits.

### SUMMARY

Disclosed herein include compositions. In some embodiments, the composition is or comprises a nucleic acid composition. The nucleic acid composition can comprise: one or more first polynucleotide(s) encoding an RNA exporter protein; one or more second polynucleotide(s) each encoding one or more cargo RNA molecule(s); and/or one or more third polynucleotide(s) encoding a fusogen. In some embodiments, the RNA exporter protein comprises: an RNA-binding domain, a membrane-binding domain, and an interaction domain capable of nucleating self-assembly. In some embodiments, a plurality of RNA exporter proteins are capable of self-assembling into lipid-enveloped nanoparticles (LNs) secreted from a sender cell in which the RNA exporter proteins are expressed, thereby generating a population of LNs comprising the fusogen and exported cargo RNA molecule(s).

Disclosed herein include compositions. In some embodiments, the composition is or comprises a population of sender cells. In some embodiments, the composition comprises: a population of sender cells comprising a nucleic acid composition disclosed herein. The population of sender cells can comprise: one or more first polynucleotide(s) encoding an RNA exporter protein; one or more second polynucleotide(s) each encoding one or more cargo RNA molecule(s); and/or one or more third polynucleotide(s) encoding a fusogen. In some embodiments, the RNA exporter protein comprises: an RNA-binding domain, a membrane-binding domain, and an interaction domain capable of nucleating self-assembly. In some embodiments, a plurality of RNA exporter proteins are capable of self-assembling into lipid-enveloped nanoparticles (LNs) secreted from a sender cell in which the RNA exporter proteins are expressed, thereby generating a population of LNs comprising the fusogen and exported cargo RNA molecule(s).

In some embodiments, the population of LNs are capable of fusing with receiver cells, thereby delivering the cargo RNA molecule(s) to said receiver cells. In some embodiments, the fusogen is capable of mediating the fusion of the lipid envelope of the LN and a lipid bilayer of a receiver cell. In some embodiments, the fusogen comprises or is derived from a SNARE protein, dynamin, an FF protein, a FAST protein, a viral fusogenic glycoprotein, or any combination thereof. In some embodiments, the viral fusogenic glycoprotein is a glycoprotein from retroviridae, herpesviridae, poxviridae, hepadnaviridae, flaviviridae, togavoridae, coronaviridae, hepatitis D virus, orthomyxoviridae, paramyxoviridae, filoviridae, rhabdoviridae, bunyaviridae, orthopoxivridae, or any combination thereof. In some embodiments, the viral fusogenic glycoprotein is selected from the group comprising HBsAg of HBV (e.g., M-, S- or L-HBsAg), E1 and/or E2 proteins of HCV, HA (hemaglutinin) and NA (neuraminidase) of Influenza, glycoprotein G of VSV, glycoprotein GP of Ebola or Marburg virus, glycoproteins Gp120 (or its CD4-binding domain) and Gp41 of lentiviruses, envelop protein (DENV E) and pre-membrane protein (prM DENV) of Dengue virus, two envelope glycoproteins of Hantaan virus, glycoprotein E2 of Chikungunya virus, glycoproteins HN and F of Newcastle virus, gp85 and gp37 of Rous sarcoma virus, and protein E and prM of Murray Valley encephalitis virus.

In some embodiments, the cargo RNA molecule(s) comprise packing signal(s). In some embodiments, the cargo RNA molecule(s) are mRNA. In some embodiments, the packing signal(s) are situated in the 5′UTR and/or 3′UTR. In some embodiments, the cargo RNA molecule(s) are transcribed from a RNA polymerase I promoter, a RNA polymerase II promoter, a RNA polymerase III promoter, a T7 promoter, a T3 promoter, or any combination thereof.

In some embodiments, the RNA exporter protein is: a chimeric fusion protein; or a multi-subunit protein. In some embodiments, the RNA exporter protein comprises two or more components configured to dimerize via dimerization domain(s). In some embodiments, the RNA binding domain is capable of binding the packing signal(s), and wherein the cargo RNA molecule(s) is specifically packaged into the LNs via interaction of the packing signal(s) with the RNA-binding domain of the RNA exporter protein. In some embodiments, the abundance of cargo RNA molecule(s) exported to the exterior of a sender cell is at least about 2-fold higher as compared to a sender cell wherein (i) the packing signal(s) are absent from the cargo RNA molecule(s) and/or (ii) the RNA exporter protein does not comprise an RNA binding domain. In some embodiments, the packing signal(s) comprise an array of at least about 2, 3, 4, 5, 6, 7, 8, 9, 10, 11, 12, 13, or 14, tandem repeats of an aptamer (e.g., a MS2 stem-loop aptamer). In some embodiments, the rate of export of cargo RNA molecule(s) is capable of being configured by varying the number of tandem repeats.

In some embodiments, the RNA exporter protein is configured to be minimally perturbative to cellular physiology and/or minimally immunogenic. In some embodiments, the RNA exporter protein comprises or is derived from one or more components of viral origin, one or more components of a non-viral compartmentalization and secretion system, and/or one or more components of de novo designed proteins. In some embodiments, the RNA exporter protein comprises a capsid protein of viral origin, optionally fused with an RNA binding protein. In some embodiments, the one or more first polynucleotide(s) encoding the RNA exporter protein comprise packing signal(s), and wherein LNs thereby comprise RNA molecules encoding the RNA exporter protein. In some embodiments, the RNA binding domain comprises or is derived from an RNA binding protein. In some embodiments, the packing signal(s) comprise a Ku binding hairpin and the RNA binding protein is Ku. In some embodiments, the packing signal(s) comprise a telomerase Sm7 binding motif and the RNA binding protein is Sm7. In some embodiments, the packing signal(s) comprise an MS2 phage operator stem-loop and the RNA binding protein is MS2 Coat Protein (MCP). In some embodiments, the packing signal(s) comprise a PP7 phage operator stem-loop and the RNA binding protein is PP7 Coat Protein (PCP). In some embodiments, the packing signal(s) comprise an SfMu phage Com stem-loop and the RNA binding protein is Com RNA binding protein. In some embodiments, the packing signal(s) comprise a PUF binding site (PBS) and the RNA binding protein is Pumilio/fem-3 mRNA binding factor (PUF). In some embodiments, the packing signal(s) comprise an MMLV packing signal (Psi) and the RNA binding protein is MMLV. In some embodiments, the RNA binding domain comprises or is derived from a catalytically inactivated programmable nuclease configured to bind the packing signal(s) (e.g., catalytically inactivated CRISPR-Cas9 or -Cas13). In some embodiments, the RNA exporter protein comprises at least a portion of a viral capsid protein (e.g., a retroviral Gag protein) and in some embodiments at least a portion of the nucleocapsid domain, matrix domain, a zinc finger domain, a p1 domain, capsid domain and/or a p1-p6 domain is removed from said viral capsid protein. In some embodiments, the nucleocapsid domain is replaced with a leucine zipper (e.g., the leucine zipper of GCN4). In some embodiments, the interaction domain comprises or is derived from a viral capsid protein (e.g., a retroviral Gag protein).

In some embodiments, the interaction domain comprises dimerization domain(s) (e.g., a leucine zipper dimerization domain from GCN4 (Zip)). In some embodiments, the dimerization domain(s) comprises or is derived from SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, a PDZ domain ligand, an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof. In some embodiments, the dimerization domain(s) is a homodimerization domain or a multimerization domain, such as, for example, a homo- or hetero-dimerizing or multimerizing leucine zipper, a PDZ domains, a SH3 domain, aGBD domain, or any combination thereof.

In some embodiments, the RNA exporter protein self-assembles to form nanocages, and wherein the LNs comprise a plurality of said nanocages. In some embodiments, the interaction domain comprises I3-01 and/or wherein the membrane binding domain comprises rat phospholipase C delta Pleckstrin Homology domain. In some embodiments, the RNA exporter protein comprises enveloped protein nanocage domain EPN24 fused with an RNA binding protein. In some embodiments, the RNA exporter protein comprises: a myristoylation motif of HIV-1 NL4-3 Gag, optionally amino acid residues 2-6; a myristoylation/palmitoylation motif of Lyn kinase, optionally amino acid residues 2-13; a Pleckstrin Homology domain of rat phospholipase Cδ; and/or a p6 domain of HIV-1 NL4-3 Gag. In some embodiments, the RNA exporter protein is configured to operate in a cell type and/or species different than the cell type and/or species in which at least one parental component of said RNA exporter protein operates. In some embodiments, the RNA exporter protein is a species-chimeric RNA exporter protein. In some embodiments, the RNA exporter protein comprises a chimeric viral capsid protein wherein the matrix domain is replaced with the matrix domain of another virus. In some embodiments, the RNA exporter protein comprises an amino acid sequence that is at least 80%, 85%, 90%, 95%, 98%, 99%, or 100% identical to any one of SEQ ID NOS: 1-24. In some embodiments, the expression of the RNA exporter protein in the sender cell(s) does not alter the endogenous transcriptome, morphology, and/or physiology of said sender cell(s).

In some embodiments, the receiver cell or sender cell is: a cell of a subject, such as a subject suffering from a disease or disorder (e.g., a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof); a cell derived from a donor; and/or an in vivo cell, an ex vivo cell, or an in situ cell. In some embodiments, upon secretion from a sender cell, the LNs are capable of distributing within one or more tissues of a subject. In some embodiments, the receiver cells are situated within one or more tissues of a subject. In some embodiments, the one or more tissues comprise adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue, fat tissue, or any combination thereof.

In some embodiments, the sender cell and/or receiver cell is a eukaryotic cell, such as a mammalian cell. In some embodiments, the mammalian cell comprises an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. In some embodiments, the stem cell comprises an embryonic stem cell, an induced pluripotent stem cell (iPSC), a hematopoietic stem/progenitor cell (HSPC), or any combination thereof.

In some embodiments, the receiver cell comprises a unique cell type and/or a unique cell state (e.g., a first cell type and/or a first cell state prior to fusing with the LNs). In some embodiments, a unique cell type and/or a unique cell state comprises a unique gene expression pattern. In some embodiments, the unique cell type and/or unique cell state comprises a unique anatomic location. In some embodiments, the unique cell type and/or the unique cell state comprises anatomically locally unique gene expression. In some embodiments, a unique cell type and/or a unique cell state is caused by hereditable, environmental, and/or idiopathic factors. In some embodiments, the unique cell type and/or the cell in the unique cell state (i) causes and/or aggravates a disease or disorder and/or (ii) is associated with the pathology of a disease or disorder. In some embodiments, the unique cell state comprises a senescent cell state induced by a tumor microenvironment. In some embodiments, the senescent cell state induced by a tumor microenvironment comprises expression of CD57, KRLG1, TIGIT, or any combination thereof. In some embodiments, the unique cell state and/or unique cell type is characterized by aberrant signaling of one or more signal transducer(s). In some embodiments, the unique cell state comprises: a physiological state (e.g., a cell cycle state, a differentiation state, a development state a metabolic state, or a combination thereof); and/or a pathological state (e.g., a disease state, a human disease state, a diabetic state, an immune disorder state, a neurodegenerative disorder state, an oncogenic state, or a combination thereof).

In some embodiments, the unique cell state and/or unique cell type is characterized by one or more of cell proliferation, stress pathways, oxidative stress, stress kinase activation, DNA damage, lipid metabolism, carbohydrate regulation, metabolic activation including Phase I and Phase II reactions, Cytochrome P-450 induction or inhibition, ammonia detoxification, mitochondrial function, peroxisome proliferation, organelle function, cell cycle state, morphology, apoptosis, DNA damage, metabolism, signal transduction, cell differentiation, cell-cell interaction and cell to non-cellular compartment.

In some embodiments, the unique cell state and/or unique cell type is characterized by one or more of acute phase stress, cell adhesion, AH-response, anti-apoptosis and apoptosis, antimetabolism, anti-proliferation, arachidonic acid release, ATP depletion, cell cycle disruption, cell matrix disruption, cell migration, cell proliferation, cell regeneration, cell-cell communication, cholestasis, differentiation, DNA damage, DNA replication, early response genes, endoplasmic reticulum stress, estogenicity, fatty liver, fibrosis, general cell stress, glucose deprivation, growth arrest, heat shock, hepatotoxicity, hypercholesterolemia, hypoxia, immunotox, inflammation, invasion, ion transport, liver regeneration, cell migration, mitochondrial function, mitogenesis, multidrug resistance, nephrotoxicity, oxidative stress, peroxisome damage, recombination, ribotoxic stress, sclerosis, steatosis, teratogenesis, transformation, disrupted translation, transport, and tumor suppression.

In some embodiments, the unique cell state and/or unique cell type is characterized by one or more of nutrient deprivation, hypoxia, oxidative stress, hyperproliferative signals, oncogenic stress, DNA damage, ribonucleotide depletion, replicative stress, and telomere attrition, promotion of cell cycle arrest, promotion of DNA-repair, promotion of apoptosis, promotion of genomic stability, promotion of senescence, and promotion of autophagy, regulation of cell metabolic reprogramming, regulation of tumor microenvironment signaling, inhibition of cell stemness, survival, and invasion.

In some embodiments, the cell type is: an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. In some embodiments, the stem cell comprises an embryonic stem cell, an induced pluripotent stem cell (iPSC), a hematopoietic stem/progenitor cell (HSPC), or any combination thereof.

In some embodiments, the receiver cell is characterized by aberrant signaling of one or more signal transducers. In some embodiments, the aberrant signaling involves an overactive signal transducer; a constitutively active signal transducer over a period of time; an active signal transducer repressor and an active signal transducer; an inactive signal transducer activator and an active signal transducer; an inactive signal transducer; an underactive signal transducer; a constitutively inactive signal transducer over a period of time; an inactive signal transducer repressor and an inactive signal transducer; and/or an active signal transducer activator and an inactive signal transducer. In some embodiments, the aberrant signaling comprises an aberrant signal of at least one signal transduction pathway regulating cell survival, cell growth, cell proliferation, cell adhesion, cell migration, cell metabolism, cell morphology, cell differentiation, apoptosis, or any combination thereof. In some embodiments, the signal transducer(s) is AKT, PI3K, MAPK, p44/42 MAP kinase, TYK2, p38 MAP kinase, PKC, PKA, SAPK, ELK, JNK, cJun, RAS, Raf, MEK 1/2, MEK 3/6, MEK 4/7, ZAP-70, LAT, SRC, LCK, ERK 1/2, Rsk 1, PYK2, SYK, PDK1, GSK3, FKHR, AFX, PLCγ, PLCy, NF-kB, FAK, CREB, αIIIβ3, FcεRI, BAD, p70S6K, STAT1, STAT2, STATS, STATS, STATE, or any combination thereof. In some embodiments, the disease or disorder is characterized by an aberrant signaling of the first transducer.

In some embodiments, one or more cargo RNA molecule(s) encode one or more payload protein(s), and wherein said payload proteins are capable of being translated upon delivery to the receiver cell(s). In some embodiments, the cargo RNA molecule(s) and/or payload protein(s) encoded by said cargo RNA molecule(s) constitute two or more components of a receiver circuit. In some embodiments, the cargo RNA(s) and/or payload(s) are capable of forming one or more receiver circuit(s) in a receiver cell. In some embodiments, the receiver circuit comprises cargo RNA molecules having molecular activity (e.g., a microRNA, an antagomir, an aptamer, and a ribozyme). In some embodiments, the receiver circuit(s) are capable of modulating cell states, cell types, and/or cell behaviors. In some embodiments, the receiver circuit(s) are configured to selectively activate cell death and/or immune recruitment to tumor cells. In some embodiments, the receiver circuit(s) are configured to detect the intracellular state of the receiver cell and classifying it as tumor or normal based on the levels or activities of relevant molecules or pathways. In some embodiments, the receiver circuit is configured to reprogram a receiver cell type and/or cell state, such as, for example, via expression of transcription factors and/or epigenetic modifiers. In some embodiments, the receiver circuit is capable of directly or indirectly inducing cell death in the presence of the aberrant signaling of one or more signal transducer(s). In some embodiments, the receiver circuit is capable of detecting aberrant signaling, an activity of a signal transducer, an activity of a signal transducer activator and/or an activity of a signal transducer repressor. In some embodiments, the detecting comprises detecting a modification selected from the group comprising phosphorylation, dephosphorylation, acetylation, methylation, acylation, glycosylation, glycosylphosphatidylinositol (GPI) anchoring, sulfation, disulfide bond formation, deamidation, ubiquitination, sumoylation, nitration of tyrosine, hydrolysis of ATP or GTP, binding of ATP or GTP, cleavage, or any combination thereof. In some embodiments, the receiver circuit(s) are capable of reprogramming a receiver cell from a first cell type and/or first cell state to a second cell type and/or second cell state. In some embodiments, the sender cell comprises one or more fourth polynucleotide(s) encoding two or more components of a sender circuit.

In some embodiments, the receiver circuit and/or sender circuit comprises one or more effector protein(s) and one or more modulator protein(s). In some embodiments, the modulator protein(s) are capable of regulating the expression, concentration, localization, stability, and/or activity the effector protein(s). In some embodiments, said regulating is based on sensing of the cell type and/or cell state of said receiver cell. In some embodiments, a modulator protein comprises a first protease, and wherein an effector protein comprises a cut site the first protease in the first protease active state is capable of cutting. In some embodiments, the first protease comprises tobacco etch virus (TEV) protease, tobacco vein mottling virus (TVMV) protease, hepatitis C virus protease (HCVP), derivatives thereof, or any combination thereof. In some embodiments, the effector protein changes from an effector inactive state to an effector active state when the first protease in the first protease active state cuts the first cut site of the effector. In some embodiments, the effector protein is changed into a first effector destabilized state, a first effector delocalized state, and/or a first effector inactivate state after the first protease in the first protease active state cuts the cut site of the effector protein. In some embodiments, the effector protein comprises a degron, wherein the first protease in the first protease active state is capable of cutting the second cut site of the effector protein to expose the degron, and wherein the degron of the effector protein being exposed changes the effector protein to an effector destabilized state. In some embodiments, the effector protein comprises a degron, wherein the first protease in the first protease active state is capable of cutting the second cut site of the effector protein to hide the degron, and wherein the degron of the effector protein being hidden changes the effector protein to an effector stabilized state. In some embodiments, the degron comprises an N-degron, a dihydrofolate reductase (DHFR) degron, a FKB protein (FKBP) degron, derivatives thereof, or any combination thereof. In some embodiments, the substrate of the effector protein(s) comprises a nucleic acid, a protein, a lipid, or any combination thereof. In some embodiments, the effector protein is capable of changing a synthetic protein circuit component of the synthetic protein circuit to (i) a synthetic protein circuit component active state; or (ii) a synthetic protein circuit component inactive state. In some embodiments, the effector protein in an effector active state is capable of: activating an endogenous signal transduction pathway; inactivating an endogenous signal transduction pathway; and/or rendering a receiver cell sensitive to a prodrug. In some embodiments, the expression, concentration, localization, stability, and/or activity the effector protein(s) is related to a number of molecules of the signal transducer in a signal transducer active state. In some embodiments, the expression and/or activity of the one or more payloads in the receiver cell is conditional on the receiver cell type and/or receiver cell state. In some embodiments, the receiver circuit and/or sender circuit is configured to be responsive to changes in: (i) cell environment, optionally cell environment comprises location relative to a target site of a subject and/or changes in the presence and/or absence of cell(s) of interest, optionally said cell(s) of interest comprise target-specific antigen(s); (ii) one or more signal transduction pathways regulating cell survival, cell growth, cell proliferation, cell adhesion, cell migration, cell metabolism, cell morphology, cell differentiation, apoptosis, or any combination thereof; (iii) input(s) of a synthetic receptor system, optionally Synthetic Notch (SynNotch) receptor, a Modular Extracellular Sensor Architecture (MESA) receptor, a synthekine, Tango, dCas9-synR, a chimeric antigen receptor, or any combination thereof; and/or (iv) T cell activity, optionally T cell activity comprises one or more of T cell simulation, T cell activation, cytokine secretion, T cell survival, T cell proliferation, CTL activity, T cell degranulation, and T cell differentiation.

In some embodiments, the effector protein in the effector active state, or the effector inactive state, is capable of changing a state of the cell, thereby treating a disease or disorder characterized by the aberrant signaling of a signal transducer. In some embodiments, the sender cell is not capable of releasing the LNs when the sender cell does not induce expression of the RNA exporter protein. In some embodiments, a sender circuit is capable of modulating the expression, concentration, localization, stability, and/or activity of the RNA exporter protein and/or the fusogen. In some embodiments, first promoter(s) are operably linked to each of the one or more first polynucleotide(s), and wherein a first promoter is capable of inducing transcription of a first polynucleotide to generate an RNA exporter transcript. In some embodiments, one or more first promoter(s) comprises a heterologous promoter element and/or an endogenous promoter element, and wherein: a heterologous promoter element is capable of being bound by an effector protein of the sender circuit (e.g., a transcription factor); and/or an endogenous promoter element is capable of being bound by an endogenous protein of a cell. In some embodiments, the RNA exporter protein is constitutively expressed by the sender cell(s). In some embodiments, the sender cell is configured such that the activation and/or degree of: fusogen expression; RNA exporter protein expression: and/or LN secretion from a sender cell is dependent on: (i) endogenous signals, optionally the local environment of the sender cell, further optionally cell-surface or soluble molecules, extracellular structures, physical or chemical properties, or combinations thereof; and/or (ii) exogenous signals, optionally light, heat, ultrasound, small molecule (e.g., transactivator), or a combination thereof. In some embodiments, one or more first polynucleotide(s) comprise one or more silencer effector binding sequences, and: wherein the silencer effector comprises a microRNA (miRNA), a precursor microRNA (pre-miRNA), a small interfering RNA (siRNA), a short-hairpin RNA (shRNA), precursors thereof, derivatives thereof, or a combination thereof, wherein said silencer effector is capable of binding the one or more silencer effector binding sequences, thereby reducing the stability of RNA exporter transcript(s); and/or wherein the expression and/or activity of the silencer effector configured to be responsive to changes in endogenous signals, and/or exogenous signals. In some embodiments, one or more first promoter(s) comprise one or more copies of a transactivator recognition sequence that a transactivator is capable of binding and wherein, in the presence of the transactivator and a transactivator-binding compound, the first promoter is capable of inducing transcription of the first polynucleotides(s). In some embodiments, the transactivator recognition sequence comprises a Tet3G binding site (TRE3G) or a ERT2-Gal4 binding site (UAS). In some embodiments, the transactivator-binding compound comprises 4-hydroxy-tamoxifen (4-OHT), Dox, derivatives thereof, or any combination thereof. In some embodiments, a transactivator recognition sequence comprises an element of an inducible promoter. In some embodiments, the inducible promoter is a tetracycline responsive promoter, a TRE promoter, a Tre3G promoter, an ecdysone responsive promoter, a cumate responsive promoter, a glucocorticoid responsive promoter, and estrogen responsive promoter, a PPAR-γ promoter, or an RU-486 responsive promoter. In some embodiments, the cargo RNA molecule(s) are capable of inducing the receiver cell to express bystander-modulating molecules, wherein bystander-modulating molecules are molecules modulating bystander cells that have not been delivered the cargo RNA molecule(s), and wherein the cargo RNA molecule(s) encode bystander-modulating molecules; or wherein the cargo RNA molecule(s), payload protein(s), and/or receiver circuit(s) are capable of inducing the receiver cell to express endogenous bystander-modulating molecules. In some embodiments, the bystander-modulating molecules comprise cytokines, morphogens, ligands, cell-surface molecules, or any combination thereof. In some embodiments, the cargo RNA molecule(s) are covalently and/or noncovalently attached to companion molecule(s). In some embodiments, the companion molecule(s) is a nucleic acid, an antibody or a portion thereof, an antibody-like molecule, an enzyme, a cell, an antigen, a small molecule, a protein, a peptide, a peptidomimetic, a sugar, a carbohydrate, a lipid, a glycan, a glycoprotein, an aptamer, or any combination thereof.

In some embodiments, a payload protein is capable of modulating the expression, concentration, localization, stability, and/or activity of the one or more endogenous proteins of a receiver cell. In some embodiments, the payload protein is a therapeutic protein or a variant thereof. In some embodiments, the therapeutic protein is configured to prevent or treat a disease or disorder of a subject, and, in some embodiments, the subject suffers from a deficiency of said therapeutic protein. In some embodiments, a payload protein comprises fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. In some embodiments, a payload protein comprises nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. In some embodiments, a payload protein comprises a CRE recombinase, GCaMP, a cell therapy component, a knock-down gene therapy component, a cell-surface exposed epitope, or any combination thereof. In some embodiments, a payload protein comprises a bispecific T cell engager (BiTE). In some embodiments, a payload protein comprises a cytokine. In some embodiments, the cytokine is selected from the group consisting of interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF, or receptor or ligand thereof. In some embodiments, a payload protein comprises a member of the TGF-β/BMP family selected from the group consisting of TGF-β1, TGF-β2, TGF-β3, BMP-2, BMP-3a, BMP-3b, BMP-4, BMP-5, BMP-6, BMP-7, BMP-8a, BMP-8b, BMP-9, BMP-10, BMP-11, BMP-15, BMP-16, endometrial bleeding associated factor (EBAF), growth differentiation factor-1 (GDF-1), GDF-2, GDF-3, GDF-5, GDF-6, GDF-7, GDF-8, GDF-9, GDF-12, GDF-14, mullerian inhibiting substance (MIS), activin-1, activin-2, activin-3, activin-4, and activin-5. In some embodiments, a payload protein comprises a member of the TNF family of cytokines selected from the group consisting of TNF-alpha, TNF-beta, LT-beta, CD40 ligand, Fas ligand, CD 27 ligand, CD 30 ligand, and 4-1 BBL. In some embodiments, a payload protein comprises a member of the immunoglobulin superfamily of cytokines selected from the group consisting of B7.1 (CD80) and B7.2 (B70). In some embodiments, a payload protein comprises an interferon. In some embodiments, the interferon is selected from interferon alpha, interferon beta, or interferon gamma. In some embodiments, a payload protein comprises a chemokine. In some embodiments, the chemokine is selected from CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13, or CXCL15. In some embodiments, a payload protein comprises an interleukin. In some embodiments, the interleukin is selected from IL-10 IL-12, IL-1, IL-6, IL-7, IL-15, IL-2, IL-18 or IL-21. In some embodiments, a payload protein comprises a tumor necrosis factor (TNF). In some embodiments, the TNF is selected from TNF-alpha, TNF-beta, TNF-gamma, CD252, CD154, CD178, CD70, CD153, or 4-1BBL. In some embodiments, a payload protein comprises a factor locally down-regulating the activity of endogenous immune cells. In some embodiments, a payload protein is capable of remodeling a tumor microenvironment and/or reducing immunosuppression at a target site of a subject.

In some embodiments, a payload protein comprises a chimeric antigen receptor (CAR) or T-cell receptor (TCR). In some embodiments, the CAR and/or TCR comprises one or more of an antigen binding domain, a transmembrane domain, and an intracellular signaling domain. In some embodiments, the intracellular signaling domain comprises a primary signaling domain, a costimulatory domain, or both of a primary signaling domain and a costimulatory domain. In some embodiments, the primary signaling domain comprises a functional signaling domain of one or more proteins selected from the group consisting of CD3 zeta, CD3 gamma, CD3 delta, CD3 epsilon, common FcR gamma (FCER1G), FcR beta (Fc Epsilon R1b), CD79a, CD79b, Fcgamma RIIa, DAP10, and DAP12, or a functional variant thereof. In some embodiments, the costimulatory domain comprises a functional domain of one or more proteins selected from the group consisting of CD27, CD28, 4-1BB (CD137), OX40, CD28-OX40, CD28-4-1BB, CD30, CD40, PD-1, ICOS, lymphocyte function-associated antigen-1 (LFA-1), CD2, CD7, LIGHT, NKG2C, B7-H3, a ligand that specifically binds with CD83, CD5, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D, or a functional variant thereof.

In some embodiments, the antigen binding domain binds a tumor antigen (e.g., a solid tumor antigen). In some embodiments, the tumor antigen is selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDG1cp (1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDG1cp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In some embodiments, the tumor antigen is selected from the group comprising CD150, 5T4, ActRIIA, B7, BMCA, CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRvIII, EGP-2, EGP-4, EPHa2, ErbB2, ErbB3, ErbB4, FBP, GD2, GD3, HER1-HER2 in combination, HER2-HER3 in combination, HERV-K, HIV-1 envelope glycoprotein gp120, HIV-1 envelope glycoprotein gp41, HLA-DR, HM1.24, HMW-MAA, Her2, Her2/neu, IGF-1R, IL-11 Ralpha, IL-13R-alpha2, IL-2, IL-22R-alpha, IL-6, IL-6R, Ia, Ii, L1-CAM, L1-cell adhesion molecule, Lewis Y, L1-CAM, MAGE A3, MAGE-A1, MART-1, MUC1, NKG2C ligands, NKG2D Ligands, NY-ESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-1, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D1), ephrinB2, epithelial tumor antigen, estrogen receptor, fetal acethycholine e receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, β2-Microglobulin, Fc Receptor-like 5 (FcRL5), or molecules expressed by HIV, HCV, HBV, or other pathogens.

In some embodiments, the antigen binding domain comprises an antibody, an antibody fragment, an scFv, a Fv, a Fab, a (Fab′)2, a single domain antibody (SDAB), a VH or VL domain, a camelid VHH domain, a Fab, a Fab′, a F(ab′)2, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof. In some embodiments, the antigen binding domain is connected to the transmembrane domain by a hinge region. In some embodiments, the transmembrane domain comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C, or a functional variant thereof. In some embodiments, the CAR or TCR further comprises a leader peptide. In some embodiments, the TCR further comprises a constant region and/or CDR4.

In some embodiments, a payload protein is an activity regulator. In some embodiments, the activity regulator is capable of reducing T cell activity. In some embodiments, the activity regulator comprises a ubiquitin ligase involved in TCR/CAR signal transduction selected from the group comprising c-CBL, CBL-B, ITCH, R F125, R F128, WWP2, or any combination thereof. In some embodiments, the activity regulator comprises a negative regulatory enzyme selected from the group comprising SHP1, SHP2, SHTP1, SHTP2, CD45, CSK, CD148, PTPN22, DGKalpha, DGKzeta, DRAK2, HPK1, HPK1, STS1, STS2, SLAT, or any combination thereof. In some embodiments, the activity regulator is a negative regulatory scaffold/adapter protein selected from the group comprising PAG, LIME, NTAL, LAX31, SIT, GAB2, GRAP, ALX, SLAP, SLAP2, DOK1, DOK2, or any combination thereof. In some embodiments, the activity regulator is a dominant negative version of an activating TCR signaling component selected from the group comprising ZAP70, LCK, FYN, NCK, VAV1, SLP76, ITK, ADAP, GADS, PLCgammal, LAT, p85, SOS, GRB2, NFAT, p50, p65, API, RAPl, CRKII, C3G, WAVE2, ARP2/3, ABL, ADAP, RIAM, SKAP55, or any combination thereof. In some embodiments, the activity regulator comprises the cytoplasmic tail of a negative co-regulatory receptor selected from the group comprising CD5, PD1, CTLA4, BTLA, LAG3, B7-H1, B7-1, CD160, TFM3, 2B4, TIGIT, or any combination thereof. In some embodiments, the activity regulator is targeted to the plasma membrane with a targeting sequence derived from LAT, PAG, LCK, FYN, LAX, CD2, CD3, CD4, CD5, CD7, CD8a, PD1, SRC, LYN, or any combination thereof. In some embodiments, the activity regulator reduces or abrogates a pathway and/or a function selected from the group comprising Ras signaling, PKC signaling, calcium-dependent signaling, NF-kappaB signaling, NFAT signaling, cytokine secretion, T cell survival, T cell proliferation, CTL activity, degranulation, tumor cell killing, differentiation, or any combination thereof.

In some embodiments, a payload protein comprises a programmable nuclease. In some embodiments, the receiver cell circuit senses correction of an aberrant locus by said programmable nuclease and reduces effector protein localization and/or activity. In some embodiments, the programmable nuclease is selected from the group comprising: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions. In some embodiments, the programmable nuclease comprises a zinc finger nuclease (ZFN) and/or transcription activator-like effector nuclease (TALEN). In some embodiments, the programmable nuclease comprises Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cash, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof. In some embodiments, the cargo RNA molecule(s) further comprise a polynucleotide encoding (i) a targeting molecule and/or (ii) a donor nucleic acid. In some embodiments, the targeting molecule is capable of associating with the programmable nuclease. In some embodiments, wherein the targeting molecule comprises single strand DNA or single strand RNA. In some embodiments, the targeting molecule comprises a single guide RNA (sgRNA).

In some embodiments, the payload protein comprises a pro-death protein capable of halting cell growth and/or inducing cell death. In some embodiments, the pro-death protein comprises cytosine deaminase, thymidine kinase, Bax, Bid, Bad, Bak, BCL2L11, p53, PUMA, Diablo/SMAC, S-TRAIL, Cas9, Cas9n, hSpCas9, hSpCas9n, HSVtk, cholera toxin, diphtheria toxin, alpha toxin, anthrax toxin, exotoxin, pertussis toxin, Shiga toxin, shiga-like toxin Fas, TNF, caspase 2, caspase 3, caspase 6, caspase 7, caspase 8, caspase 9, caspase 10, caspase 11, caspase 12, purine nucleoside phosphorylase, or any combination thereof. In some embodiments, the pro-death protein is capable of halting cell growth and/or inducing cell death in the presence of a pro-death agent. In some embodiments, the pro-death protein comprises Caspase-9 and the pro-death agent comprises AP1903. In some embodiments, the pro-death protein comprises HSV thymidine kinase (TK) and the pro-death agent Ganciclovir (GCV), Ganciclovir elaidic acid ester, Penciclovir (PCV), Acyclovir (ACV), Valacyclovir (VCV), (E)-5-(2-bromovinyl)-2′-deoxyuridine (BVDU), Zidovuline (AZT), and/or 2′-exo-methanocarbathymidine (MCT). In some embodiments, the pro-death protein comprises Cytosine Deaminase (CD) and the pro-death agent comprises 5-fluorocytosine (5-FC). In some embodiments, the pro-death protein comprises Purine nucleoside phosphorylase (PNP) and the pro-death agent comprises 6-methylpurine deoxyriboside (MEP) and/or fludarabine (FAMP). In some embodiments, the pro-death protein comprises a Cytochrome p450 enzyme (CYP) and the pro-death agent comprises Cyclophosphamide (CPA), Ifosfamide (IFO), and/or 4-ipomeanol (4-IM). In some embodiments, the pro-death protein comprises a Carboxypeptidase (CP) and the pro-death agent comprises 4-[(2-chloroethyl)(2-mesyloxyethyl)amino]benzoyl-L-glutamic acid (CMDA), Hydroxy- and amino-aniline mustards, Anthracycline glutamates, and/or Methotrexate α-peptides (MTX-Phe). In some embodiments, the pro-death protein comprises Carboxylesterase (CE) and the pro-death agent comprises Irinotecan (IRT), and/or Anthracycline acetals. In some embodiments, the pro-death protein comprises Nitroreductase (NTR) and the pro-death agent comprises dinitroaziridinylbenzamide CB1954, dinitrobenzamide mustard SN23862, 4-Nitrobenzyl carbamates, and/or Quinones. In some embodiments, the pro-death protein comprises Horse radish peroxidase (HRP) and the pro-death agent comprises Indole-3-acetic acid (IAA) and/or 5-Fluoroindole-3-acetic acid (FIAA). In some embodiments, the pro-death protein comprises Guanine Ribosyltransferase (XGRTP) and the pro-death agent comprises 6-Thioxanthine (6-TX). In some embodiments, the pro-death protein comprises a glycosidase enzyme and the pro-death agent comprises HM1826 and/or Anthracycline acetals. In some embodiments, the pro-death protein comprises Methionine-α,γ-lyase (MET) and the pro-death agent comprises Selenomethionine (SeMET). In some embodiments, the pro-death protein comprises thymidine phosphorylase (TP) and the pro-death agent comprises 5′-Deoxy-5-fluorouridine (5′-DFU).

In some embodiments, a payload protein is a cellular reprogramming factor capable of converting an at least partially differentiated cell to a less differentiated cell (e.g., Oct-3, Oct-4, Sox2, c-Myc, Klf4, Nanog, Lin28, ASCL1, MYT1L, TBX3b, SV40 large T, hTERT, miR-291, miR-294, miR-295, or any combinations thereof). In some embodiments, a payload protein is a cellular reprogramming factor capable of differentiating a given cell into a desired differentiated state (e.g., nerve growth factor (NGF), fibroblast growth factor (FGF), interleukin-6 (IL-6), bone morphogenic protein (BMP), neurogenin3 (Ngn3), pancreatic and duodenal homeobox 1 (Pdx1), Mafa, or any combination thereof). In some embodiments, a payload protein comprises an agonistic or antagonistic antibody or antigen-binding fragment thereof specific to a checkpoint inhibitor or checkpoint stimulator molecule (e.g., PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, and/or TIM-3). In some embodiments, the one or more payloads comprise a secretion tag. In some embodiments, the secretion tag is selected from the group comprising AbnA, AmyE, AprE, BglC, BglS, Bpr, Csn, Epr, Ggt, GlpQ, HtrA, LipA, LytD, MntA, Mpr, NprE, OppA, PbpA, PbpX, Pel, PelB, PenP, PhoA, PhoB, PhoD, PstS, TasA, Vpr, WapA, WprA, XynA, XynD, YbdN, Ybxl, YcdH, YclQ, YdhF, YdhT, YfkN, YflE, YfmC, Yfnl, YhcR, YlqB, YncM, YnfF, YoaW, YocH, YolA, YqiX, Yqxl, YrpD, YrpE, YuaB, Yurl, YvcE, YvgO, YvpA, YwaD, YweA, YwoF, YwtD, YwtF, YxaLk, YxiA, and YxkC. In some embodiments, a payload protein comprises a constitutive signal peptide for protein degradation (e.g., PEST). In some embodiments, a payload protein comprises a nuclear localization signal (NLS) or a nuclear export signal (NES). In some embodiments, a payload protein comprises a dosage indicator protein. In some embodiments, the dosage indicator protein is detectable. In some embodiments, the dosage indicator protein comprises green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof.

In some embodiments, the payload protein(s) are configured to reduce their expression, localization, stability, and/or activity in sender cells. In some embodiments, the sender cells express a second protease, wherein payload protein(s) comprises a cut site the second protease in the second protease active state is capable of cutting to reduce the stability, localization, and/or activity of the payload protein(s). In some embodiments, receiver cells does not comprise the second protease. In some embodiments, the second protease comprises tobacco etch virus (TEV) protease, tobacco vein mottling virus (TVMV) protease, hepatitis C virus protease (HCVP), derivatives thereof, or any combination thereof. In some embodiments, the second protease cutting the cut site exposes a degron. In some embodiments, the degron comprises an N-degron, a dihydrofolate reductase (DHFR) degron, a FKB protein (FKBP) degron, derivatives thereof, or any combination thereof. In some embodiments, one or more of the payload protein(s) is a degron fusion protein comprising a degron capable of binding a degron stabilizing molecule, and wherein the degron fusion protein changes from a destabilized state to a stabilized state when the degron binds to the degron stabilizing molecule, and wherein the degron stabilizing molecule is: an endogenous molecule of a receiver cell; a molecule absent in a sender cell; is a molecule specific to a cell type; is a molecule specific to a disease or disorder; and/or is a synthetic protein circuit component. In some embodiments, one or more of the payload protein(s) is a conditionally stable fusion protein comprising a stabilizing molecule binding domain capable of binding a stabilizing molecule, and wherein the conditionally stable fusion protein changes from a destabilized state to a stabilized state when the stabilizing molecule binding domain binds to the stabilizing molecule, and wherein the stabilizing molecule is: an endogenous molecule of a receiver cell; a molecule absent in a sender cell; is a molecule specific to a cell type; is a molecule specific to a disease or disorder; and/or is a synthetic protein circuit component. In some embodiments, the stabilizing molecule binding domain comprises a Camel Ig, Ig NAR, Fab fragments, Fab′ fragments, F(ab)′2 fragments, F(ab)′3 fragments, Fv, single chain Fv antibody (“scFv”), bis-scFv, (scFv)2, minibody, diabody, triabody, tetrabody, disulfide stabilized Fv protein (“dsFv”), single-domain antibody (sdAb), or any combination thereof.

In some embodiments, a payload protein comprises one or more receptors and/or a targeting moiety configured to bind a component of a target site of a subject. In some embodiments, the sender sends are configured to travel to and/or accumulate at a target site of a subject. In some embodiments, the LNs comprise one or more targeting moieties configured to bind: (i) a target site of a subject; and/or (ii) a first antigen of target receiver cells. In some embodiments, a target site of a subject: comprises target receiver cells; and/or is a site of disease or disorder or is proximate to a site of a disease or disorder. In some embodiments, the one or more targeting moieties are selected from the group comprising mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, and an RGD peptide or RGD peptide mimetic. In some embodiments, the one or more targeting moieties comprise one or more of the following: an antibody or antigen-binding fragment thereof, a peptide, a polypeptide, an enzyme, a peptidomimetic, a glycoprotein, a lectin, a nucleic acid, a monosaccharide, a disaccharide, a trisaccharide, an oligosaccharide, a polysaccharide, a glycosaminoglycan, a lipopolysaccharide, a lipid, a vitamin, a steroid, a hormone, a cofactor, a receptor, a receptor ligand, and analogs and derivatives thereof.

In some embodiments, the one or more targeting moieties are configured to bind one or more of the following: CD3, CD4, CD5, CD6, CD7, CD8, CD9, CD10, CD11a, CD11b, CD11c, CD12w, CD14, CD15, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD46, CD47, CD48, CD49b, CD49c, CD51, CD52, CD53, CD54, CD55, CD56, CD58, CD59, CD61, CD62E, CD62L, CD62P, CD63, CD66, CD68, CD69, CD70, CD72, CD74, CD79, CD79a, CD79b, CD80, CD81, CD82, CD83, CD86, CD87, CD88, CD89, CD90, CD91, CD95, CD96, CD98, CD100, CD103, CD105, CD106, CD109, CD117, CD120, CD125, CD126, CD127, CD133, CD134, CD135, CD137, CD138, CD141, CD142, CD143, CD144, CD147, CD151, CD147, CD152, CD154, CD156, CD158, CD163, CD166, CD168, CD174, CD180, CD184, CDw186, CD194, CD195, CD200, CD200a, CD200b, CD209, CD221, CD227, CD235a, CD240, CD262, CD271, CD274, CD276 (B7-H3), CD303, CD304, CD309, CD326, 4-1BB, 5 AC, 5T4 (Trophoblast glycoprotein, TPBG, 5T4, Wnt-Activated Inhibitory Factor 1 or WAIF1), Adenocarcinoma antigen, AGS-5, AGS-22M6, Activin receptor like kinase 1, AFP, AKAP-4, ALK, Alpha integrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), B-lymphoma cell, bcr-abl, Bombesin, BORIS, C5, C242 antigen, CA125 (carbohydrate antigen 125, MUC16), CA-IX (CAIX, carbonic anhydrase 9), CALLA, CanAg, Canis lupus familiaris IL31, Carbonic anhydrase IX, Cardiac myosin, CCL11 (C—C motif chemokine 11), CCR4 (C—C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, CRIPTO, FCSF1R (Colony stimulating factor 1 receptor, CD 115), CSF2 (colony stimulating factor 2, Granulocyte-macrophage colony-stimulating factor (GM-CSF)), CTLA4 (cytotoxic T-lymphocyte-associated protein 4), CTAA16.88 tumor antigen, CXCR4 (CD 184), C—X—C chemokine receptor type 4, cyclic ADP ribose hydrolase, Cyclin B1, CYP1B1, Cytomegalovirus, Cytomegalovirus glycoprotein B, Dabigatran, DLL4 (delta-like—ligand 4), DPP4 (Dipeptidyl-peptidase 4), DR5 (Death receptor 5), E. coli Shiga toxin type-1, E. coli Shiga toxin type-2, ED-B, EGFL7 (EGF-like domain-containing protein 7), EGFR, EGFRII, EGFRvIII, Endoglin (CD 105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen 1.F protein of respiratory syncytial virus, Frizzled receptor, Fucosyl GM1, GD2 ganglioside, G-28 (a cell surface antigen glycolipid), GD3 idiotype, GloboH, Glypican 3, N-glycolylneuraminic acid, GM3, GMCSF receptor a-chain, Growth differentiation factor 8, GP100, GPNMB (Transmembrane glycoprotein NMB), GUCY2C (Guanylate cyclase 2C, guanylyl cyclase C (GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-γ, Influenza hemagglutinin, IgE, IgE Fc region, IGHE, IL-1, IL-2 receptor (interleukin 2 receptor), IL-4, IL-5, IL-6, IL-6R (interleukin 6 receptor), IL-9, IL-10, IL-12, IL-13, IL-17, IL-17A, IL-20, IL-22, IL-23, IL31RA, ILGF2 (Insulin-like growth factor 2), Integrins (α4, αₙβ₃, ανβ3, α₄β₇, α5β1, α6β4, α7β7, α11β3, α5β5, ανβ5), Interferon gamma-induced protein, ITGA2, ITGB2, KIR2D, LCK, Le, Legumain, Lewis-Y antigen, LFA-l (Lymphocyte function-associated antigen 1, CD11a), LHRH, LINGO-1, Lipoteichoic acid, LIV1A, LMP2, LTA, MAD-CT-1, MAD-CT-2, MAGE-1, MAGE-2, MAGE-3, MAGE A1, MAGE A3, MAGE 4, MARTI, MCP-1, MIF (Macrophage migration inhibitory factor, or glycosylation inhibiting factor (GIF)), MS4A1 (membrane-spanning 4-domains subfamily A member 1), MSLN (mesothelin), MUCl (Mucin 1, cell surface associated (MUC1) or polymorphic epithelial mucin (PEM)), MUC1-KLH, MUC16 (CA125), MCPl (monocyte chemotactic protein 1), MelanA/MART1, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES 1, P21, p53 nonmutant, P97, Page 4, PAP, Paratope of anti-(N-glycolylneuraminic acid), PAX3, PAX5, PCSK9, PDCD1 (PD-1, Programmed cell death protein 1, CD279), PDGF-Ra (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-a, TGF-β (Transforming growth factor beta), TGF-βI, TGF-β2 (Transforming growth factor-beta 2), Tie (CD202b), Tie2, TIM-1 (CDX-014), Tn, TNF, TNF-a, TNFRSF8, TNFRSF10B (tumor necrosis factor receptor superfamily member 10B), TNFRSF13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TRP-2, Tyrosinase, VCAM-1 (CD 106), VEGF, VEGF-A, VEGF-2 (CD309), VEGFR-1, VEGFR2, or vimentin, WT1, XAGE 1, or cells expressing any insulin growth factor receptors, or any epidermal growth factor receptors. In some embodiments, the fusogen is configured to bind one or more receiver cells of a subject.

The composition can comprise: a bridge protein. In some embodiments, a bridge protein comprises a fusogen-binding domain and a first antigen-binding domain. In some embodiments, a first antigen-binding moiety of the bridge protein is capable of binding a first antigen on a surface of a target receiver cell. In some embodiments, a fusogen-binding domain of the bridge protein is capable of binding a fusogen on a surface of a LN. In some embodiments, the LN is capable of fusing with a receiver cell when the first antigen-binding moiety is bound to the first antigen and the fusogen-binding domain is bound to the fusogen. In some embodiments, the one or more of one or more of the first polynucleotide(s), the second polynucleotide(s), the third polynucleotide(s), and the fourth polynucleotide(s), is between about 30 and 100000 nucleotides in length. In some embodiments, the payload protein(s) is between about 30 amino acids and 3000 amino acids in length.

In some embodiments, one or more of one or more of the first polynucleotide(s), the second polynucleotide(s), the third polynucleotide(s), and the fourth polynucleotide(s), comprise: a 5′UTR and/or a 3′UTR; a tandem gene expression element selected from the group an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof; and/or a transcript stabilization element (e.g., woodchuck hepatitis post-translational regulatory element (WPRE), bovine growth hormone polyadenylation (bGH-polyA) signal sequence, human growth hormone polyadenylation (hGH-polyA) signal sequence, or any combination thereof).

In some embodiments, one or more of one or more of the first polynucleotide(s), the second polynucleotide(s), the third polynucleotide(s), and the fourth polynucleotide(s), are operably connected to a promoter selected from the group comprising: a minimal promoter, optionally TATA, miniCMV, and/or miniPromo; a ubiquitous promoter; a tissue-specific promoter and/or a lineage-specific promoter; and/or a ubiquitous promoter (e.g., a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof).

In some embodiments, the nucleic acid composition and/or cargo RNA molecule(s) is configured to enhance stability, durability, and/or expression level, optionally a 5′ untranslated region (UTR), a 3′ UTR, and/or a 5′ cap; optionally one or more modified nucleotides, further optionally selected from the group comprising pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine; and/or optionally a modified nucleotide in place of one or more uridines, optionally the modified nucleoside is selected from pseudouridine (Ψ), N1-methyl-pseudouridine (m 1Ψ), and 5-methyl-uridine (m5U).

In some embodiments, the nucleic acid composition is complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the nucleic acid composition is, comprises, or further comprises, one or more vectors. In some embodiments, at least one of the one or more vectors is a viral vector, a plasmid, a transposable element, a naked DNA vector, a lipid nanoparticle (LNP), or any combination thereof. In some embodiments, the viral vector is an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. In some embodiments, the transposable element is piggybac transposon or sleeping beauty transposon. In some embodiments, the polynucleotide(s) encoding the RNA exporter protein, the fusogen, and/or the cargo RNA molecule(s) are comprised in the one or more vectors. In some embodiments, the polynucleotide(s) encoding the RNA exporter protein, the fusogen, and/or the cargo RNA molecule(s) are comprised in the same vector and/or different vectors. In some embodiments, the polynucleotide(s) encoding the RNA exporter protein, the fusogen, and/or the cargo RNA molecule(s) are situated on the same nucleic acid and/or different nucleic acids. In some embodiments, the payload protein(s), fusogen, and/or RNA exporter protein are configured to exhibit minimal immunogenicity in a subject, and in some embodiments can be derived from commensal viruses or endogenous viruses and/or can be novo designed proteins and/or and humanized.

Disclosed herein include pharmaceutical compositions. In some embodiments, the pharmaceutical composition comprises: a composition provided herein (e.g., a nucleic acid composition, a population of sender cells), wherein the pharmaceutical composition further comprises one or more pharmaceutically acceptable carriers, diluents and/or excipients.

Disclosed herein include systems for delivery of RNA molecules from sender cells to receiver cells. In some embodiments, the system comprises: an RNA exporter protein provided herein; cargo RNA molecule(s) provided herein; and a fusogen provided herein.

Disclosed herein include populations of lipid-enveloped nanoparticles (LNs). In some embodiments, the population of LNs comprises: an RNA exporter protein provided herein; cargo RNA molecule(s) provided herein; and a fusogen provided herein. The population of the LNs can be derived from expression of a nucleic acid composition provided herein.

Disclosed herein include methods of treating or preventing a disease or disorder in a subject in need thereof. In some embodiments, the method comprises: administering to the subject an effective amount of a nucleic acid composition disclosed herein, a pharmaceutical composition disclosed herein, or the sender cells disclosed herein, thereby treating or preventing the disease or disorder in the subject.

In some embodiments, administering comprises: (i) isolating one or more cells from the subject; (ii) contacting (e.g,. transfecting) said one or more cells with a nucleic acid composition disclosed herein, thereby generating sender cells; and (iii) administering the one or more sender cells into a subject after the contacting step. The method can comprise: administering to the subject an effective amount of a transactivator, a bridge protein, a pro-death agent, or any combination thereof. In some embodiments, the sender sends are configured to travel to and/or accumulate at a target site of a subject. In some embodiments, method comprises administering a bridge protein to a subject. In some embodiments, the sender cell is capable of expressing a bridge protein. The method can comprise: applying a stimulus to a target site of the subject configured to induce RNA exporter expression, fusogen expression, and/or LN secretion at said target site (e.g., applying thermal energy to a target site of the subject sufficient to increase the local temperature of the target site). In some embodiments, applying thermal energy to a target site of the subject comprises the application of one or more of focused ultrasound (FUS), magnetic hyperthermia, microwaves, infrared irradiation, liquid-based heating (e.g., intraperitoneal chemotherapy (HIPEC)), and contact heating.

In some embodiments, the target site comprises a site of disease or disorder or is proximate to a site of a disease or disorder. In some embodiments, the target site a tissue. In some embodiments: the tissue comprises adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue and/or fat tissue; tissue is inflamed tissue; and/or the tissue comprises (i) grade I, grade II, grade III or grade IV cancerous tissue; (ii) metastatic cancerous tissue; (iii) mixed grade cancerous tissue; (iv) a sub-grade cancerous tissue; (v) healthy or normal tissue; and/or (vi) cancerous or abnormal tissue.

In some embodiments, at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the plurality of LNs and/or payload molecules are released at the target site. In some embodiments, less than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the plurality of LNs and/or payload molecules are released at a location other than the target site. In some embodiments, the ratio of the concentration of sender cells, receiver cells, LNs, and/or payload molecules at the subject's target site to the concentration of sender cells, receiver cells, LNs, and/or payload molecules in subject's blood, serum, or plasma is about 2:1 to about 3000:1, about 2:1 to about 2000:1, about 2:1 to about 1000:1, or about 2:1 to about 600:1. In some embodiments, the disease or disorder is a blood disease, an immune disease, a neurological disease or disorder, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, a solid tumor, or any combination thereof.

In some embodiments, the disease or disorder is an infectious disease selected from the group consisting of an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-CoV-2), Creutzfeldt-Jacob Disease/transmissible spongiform encephalopathy, Cryptosporidiosis (Crypto), Cyclosporiasis, Dengue 1,2,3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru (EV-D68), Giardiasis, Glanders, Gonococcal Infection, Granuloma inguinale, Haemophilus influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Meliodosis, Meningitis (Viral), Meningococcal Disease (Meningitis (Bacterial)), Middle East Respiratory Syndrome Coronavirus (MERS-CoV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphylococcal Infection Methicillin-resistant (MRSA), Staphylococcal Food Poisoning Enterotoxin B Poisoning (Staph Food Poisoning), Staphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichinosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection.

In some embodiments, the disease is associated with expression of a tumor-associated antigen. In some embodiments, the disease associated with expression of a tumor antigen-associated is selected from the group consisting of a proliferative disease, a precancerous condition, a cancer, and a non-cancer related indication associated with expression of the tumor antigen. In some embodiments, the cancer is selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments, the cancer is a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

In some embodiments, administering comprises aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof. The method can comprise: administering one or more additional agents to the subject. In some embodiments, the one or more additional agents comprise: a protein phosphatase inhibitor, a kinase inhibitor, a cytokine, an inhibitor of an immune inhibitory molecule, and/or or an agent that decreases the level or activity of a TREG cell; an immune modulator, an anti-metastatic, a chemotherapeutic, a hormone or a growth factor antagonist, an alkylating agent, a TLR agonist, a cytokine antagonist, a cytokine antagonist, or any combination thereof; and/or an agonistic or antagonistic antibody specific to a checkpoint inhibitor or checkpoint stimulator molecule (e.g., PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, TIM-3).

Disclosed herein include methods for determining sender cell-receiver cell dynamics. In some embodiments, the method comprises: contacting a population of sender cell(s) disclosed herein with a population of receiver cells, wherein the exported cargo RNA molecule(s) are thereby delivered to the receiver cells, and wherein the exported cargo RNA molecule(s) comprise one or more cell barcodes identifying the sender cell from which the exported cargo RNA molecule(s) are derived; and obtaining sequence information of the plurality of exported cargo RNA molecule(s), or products thereof, in each of the receiver cells to determine sender cell-receiver cell dynamics.

In some embodiments, the contacting is in vivo (e.g., within a tissue). In some embodiments, obtaining sequence information comprises sequencing or in situ hybridization In some embodiments, obtaining sequence information comprises, for each receiver cell, detecting the presence and/or amount of cell barcodes associated with each sender cell. In some embodiments, sender cell-receiver cell dynamics comprises: detecting of sender cell-receiver cell proximity; determining the relative positions of sender cells and receiver cells within a tissue and/or determining probing molecular transport rates within said tissue; and/or determining a three-dimensional physical map of the receiver cell population, sender cell population, and/or tissue.

### BRIEF DESCRIPTION OF THE DRAWINGS

FIGS. 1A-1H depict non-limiting exemplary schematics and data related to engineered viral RNA exporters efficiently and specifically packaging, secreting, and protecting RNA. FIG. 1A depicts a non-limiting exemplary schematic of RNA export via secretion of LNs. FIG. 1B depicts non-limiting exemplary schematics of viral RNA exporter designs. Left, exporter based on Moloney Murine Leukemia Virus (MMLV) Gag capsid protein. FIG. 1C depicts data related to negative stain transmission electron microscopy showing secretion of enveloped particles from HEK293T cells transfected with plasmids encoding RNA exporters. FIG. 1D depicts data related to the efficiency and specificity of RNA export to supernatant determined by reverse transcription followed by quantitative PCR (RT-qPCR). FIG. 1E depicts a non-limiting exemplary schematic of an experiment to characterize RNA exporters by sequencing. FIG. 1F depicts data related to the specificity of RNA export determined by sequencing. FIG. 1G depicts data related to the quantification of endogenous cellular RNA among supernatant RNA. FIG. 1H depicts data related to RNA exporters packaging and protecting RNA from degradation.

FIGS. 2A-2D depict non-limiting exemplary schematics and data related to the design and characterization of engineered viral RNA exporters. FIG. 2A depicts non-limiting exemplary domain architectures of viral RNA exporters. Left to right, N- to C-terminus. Colors indicate distinct protein domains. FIG. 2B depicts data related to the quantification of RNA export by viral RNA exporters with various architectures using reverse transcription followed by quantitative PCR (RT-qPCR). FIG. 2C depicts data related to the quantification of RNA export system components in cellular RNA by RT-qPCR, using the same specimens as (B). FIG. 2D depicts data related to technical characteristics of RT-qPCR assay for RNA export to supernatant, indicating that assay faithfully and reproducibly measures RNA, not DNA, abundance.

FIGS. 3A-3G depict non-limiting exemplary schematics and data related to synthetic non-viral RNA exporters efficiently and specifically packaging, secreting, and protecting RNA. FIG. 3A depicts a non-limiting exemplary schematic of synthetic non-viral RNA exporter design. FIG. 3B depicts a non-limiting exemplary protein design model of an RNA-exporting nanocage. FIG. 3C depicts data related to negative stain transmission electron microscopy showing secretion of enveloped particles from HEK293T cells transfected with plasmids encoding RNA exporter. FIG. 3D depicts data related to the efficiency and specificity of RNA export to supernatant determined by reverse transcription followed by quantitative PCR (RT-qPCR). FIG. 3E depicts data related to the specificity of RNA export determined by sequencing. FIG. 3F depicts data related to the quantification of endogenous cellular RNA among supernatant RNA showing specific RNA export, similar to engineered viral RNA exporters. FIG. 3G depicts data related to the RNA exporters packaging and protecting RNA from degradation.

FIGS. 4A-4C depict non-limiting exemplary schematics and data related to the design and characterization of synthetic RNA exporters. FIG. 4A depicts non-limiting exemplary domain architectures of synthetic RNA exporters. FIG. 4B depicts a non-limiting exemplary schematic of assay for RNA export using stable reporter cell line. FIG. 4C depicts data related to the quantification of RNA export by synthetic RNA exporters with various architectures using RT-qPCR.

FIGS. 5A-5E depict data related to physical and functional features of RNA export systems. FIG. 5A depicts data related to the quantification of RNA export by RT-qPCR revealing that the rate of RNA export depends on copy number of the packaging signal, which is a tandem array of MS2 aptamers. FIG. 5B depicts data related to the quantification of RNA export by RT-qPCR reveals that RNA exporters can package their own RNA. FIG. 5C depicts data related to negative stain transmission electron microscopy showing the lack of enveloped particles in supernatant collected from HEK293T cells transfected with plasmids encoding the reporter, but not the exporter. FIG. 5D depicts data related to the physical size of RNA export particles, as determined by dynamic light scattering. FIG. 5E depicts data related to the quantification of RNA export by RT-qPCR from mouse embryonic stem cells (mESCs).

FIGS. 6A-6C depict data showing RNA exporters are non-toxic and do not perturb cellular morphology and transcriptome. FIG. 6A depicts non-limiting exemplary images of HEK293T cells transfected with RNA exporters and reporters. FIG. 6B depicts data related to the quantification of toxicity of RNA exporter expression in HEK293T cells using flow cytometry with dead stain (ethidium homodimer-1). FIG. 6C depicts data related to differential expression analysis of cellular transcriptomes of cells transfected with and without exporters.

FIGS. 7A-7C depict non-limiting exemplary schematics and data related to RNA export enabling cell-to-cell delivery of therapeutic gene circuits. FIG. 7A depicts a non-limiting exemplary schematic of in situ control of living cells by cell-to-cell delivery of RNA. FIG. 7B depicts a non-limiting exemplary design of synthetic RNA delivery system. FIG. 7C depicts a demonstration of cell-to-cell delivery of functional mRNA.

FIG. 8 depicts data related to characterization and optimization of cell-to-cell RNA delivery system.

FIG. 9 depicts a non-limiting exemplary schematic of export systems provided herein.

FIGS. 10A-10B depict non-limiting exemplary schematics and data related to demonstration of cell-to-cell transfer of RNA circuits. FIG. 10A depicts a non-limiting exemplary schematic of one exemplary system provided herein. FIG. 10B depicts data related to flow cytometry revealing expression of RNA circuits transferred from sender to receiver cells.

FIGS. 11A-11B depict data demonstrating the prevention of cargo expression in sender cells. Engineered HEK293T sender cells that constitutively express TEV Protease (TEVP) were transfected with DNA plasmid encoding RNA exporter (Gag-MCP), fusogen (VSVG), and RNA cargo either having (FIG. 11A) or lacking (FIG. 11B) a protease-regulatable N-terminal degron (N-Deg-mCherry-MS2×12 and mCherry-MS2×12, respectively).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include compositions. In some embodiments, the composition is or comprises a nucleic acid composition. The nucleic acid composition can comprise: one or more first polynucleotide(s) encoding an RNA exporter protein; one or more second polynucleotide(s) each encoding one or more cargo RNA molecule(s); and/or one or more third polynucleotide(s) encoding a fusogen. In some embodiments, the RNA exporter protein comprises: an RNA-binding domain, a membrane-binding domain, and an interaction domain capable of nucleating self-assembly. In some embodiments, a plurality of RNA exporter proteins are capable of self-assembling into lipid-enveloped nanoparticles (LNs) secreted from a sender cell in which the RNA exporter proteins are expressed, thereby generating a population of LNs comprising the fusogen and exported cargo RNA molecule(s).

Disclosed herein include compositions. In some embodiments, the composition is or comprises a population of sender cells. In some embodiments, the composition comprises: a population of sender cells comprising a nucleic acid composition disclosed herein. The population of sender cells can comprise: one or more first polynucleotide(s) encoding an RNA exporter protein; one or more second polynucleotide(s) each encoding one or more cargo RNA molecule(s); and/or one or more third polynucleotide(s) encoding a fusogen. In some embodiments, the RNA exporter protein comprises: an RNA-binding domain, a membrane-binding domain, and an interaction domain capable of nucleating self-assembly. In some embodiments, a plurality of RNA exporter proteins are capable of self-assembling into lipid-enveloped nanoparticles (LNs) secreted from a sender cell in which the RNA exporter proteins are expressed, thereby generating a population of LNs comprising the fusogen and exported cargo RNA molecule(s). The fusogen and/or targeting moieties provided herein can be passively incorporated into the LNs. The fusogen and/or targeting moieties can be specifically configured to be present in the LNs (e.g., via binding the RNA exporter protein).

Disclosed herein include pharmaceutical compositions. In some embodiments, the pharmaceutical composition comprises: a composition provided herein (e.g., a nucleic acid composition, a population of sender cells), wherein the pharmaceutical composition further comprises one or more pharmaceutically acceptable carriers, diluents and/or excipients.

Disclosed herein include systems for delivery of RNA molecules from sender cells to receiver cells. In some embodiments, the system comprises: an RNA exporter protein provided herein; cargo RNA molecule(s) provided herein; and a fusogen provided herein.

Disclosed herein include populations of lipid-enveloped nanoparticles (LNs). In some embodiments, the population of LNs comprises: an RNA exporter protein provided herein; cargo RNA molecule(s) provided herein; and a fusogen provided herein. The population of the LNs can be derived from expression of a nucleic acid composition provided herein.

Disclosed herein include methods of treating or preventing a disease or disorder in a subject in need thereof. In some embodiments, the method comprises: administering to the subject an effective amount of a nucleic acid composition disclosed herein, a pharmaceutical composition disclosed herein, or the sender cells disclosed herein, thereby treating or preventing the disease or disorder in the subject.

Disclosed herein include methods for determining sender cell-receiver cell dynamics. In some embodiments, the method comprises: contacting a population of sender cell(s) disclosed herein with a population of receiver cells, wherein the exported cargo RNA molecule(s) are thereby delivered to the receiver cells, and wherein the exported cargo RNA molecule(s) comprise one or more cell barcodes identifying the sender cell from which the exported cargo RNA molecule(s) are derived; and obtaining sequence information of the plurality of exported cargo RNA molecule(s), or products thereof, in each of the receiver cells to determine sender cell-receiver cell dynamics.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). For purposes of the present disclosure, the following terms are defined below.

As used herein, the terms “nucleic acid” and “polynucleotide” are interchangeable and refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The term “vector” as used herein, can refer to a vehicle for carrying or transferring a nucleic acid. Non-limiting examples of vectors include plasmids and viruses (for example, AAV viruses).

The term “construct,” as used herein, refers to a recombinant nucleic acid that has been generated for the purpose of the expression of a specific nucleotide sequence(s), or that is to be used in the construction of other recombinant nucleotide sequences.

As used herein, the term “plasmid” refers to a nucleic acid that can be used to replicate recombinant DNA sequences within a host organism. The sequence can be a double stranded DNA.

The term “element” refers to a separate or distinct part of something, for example, a nucleic acid sequence with a separate function within a longer nucleic acid sequence. The term “regulatory element” and “expression control element” are used interchangeably herein and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see, e.g., Lewin, “Genes V” (Oxford University Press, Oxford) pages 847-873). Exemplary regulatory elements in prokaryotes include promoters, operator sequences and a ribosome binding sites. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription, regardless of the distance or orientation of the enhancer relative to the start site of transcription.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

As used herein, a “subject” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human.

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

As used herein, the term “effective amount” refers to an amount sufficient to effect beneficial or desirable biological and/or clinical results.

“Pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™. Auxiliary, stabilizer, emulsifier, lubricant, binder, pH adjustor controller, isotonic agent and other conventional additives may also be added to the carriers.

The term “autologous” shall be given its ordinary meaning, and shall also refer to any material derived from the same individual to whom it is later to be re-introduced into the individual.

The term “allogeneic” shall be given its ordinary meaning, and shall also refer to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “stimulation,” shall be given its ordinary meaning, and shall also refer to a primary response induced by binding of a stimulatory molecule (e.g., a TCR/CD3 complex or CAR) with its cognate ligand (or tumor antigen in the case of a CAR) thereby mediating a signal transduction event, such as, but not limited to, signal transduction via the TCR/CD3 complex or signal transduction via the appropriate NK receptor or signaling domains of the CAR. Stimulation can mediate altered expression of certain molecules.

### Cell-to-Cell Delivery of RNA Circuits

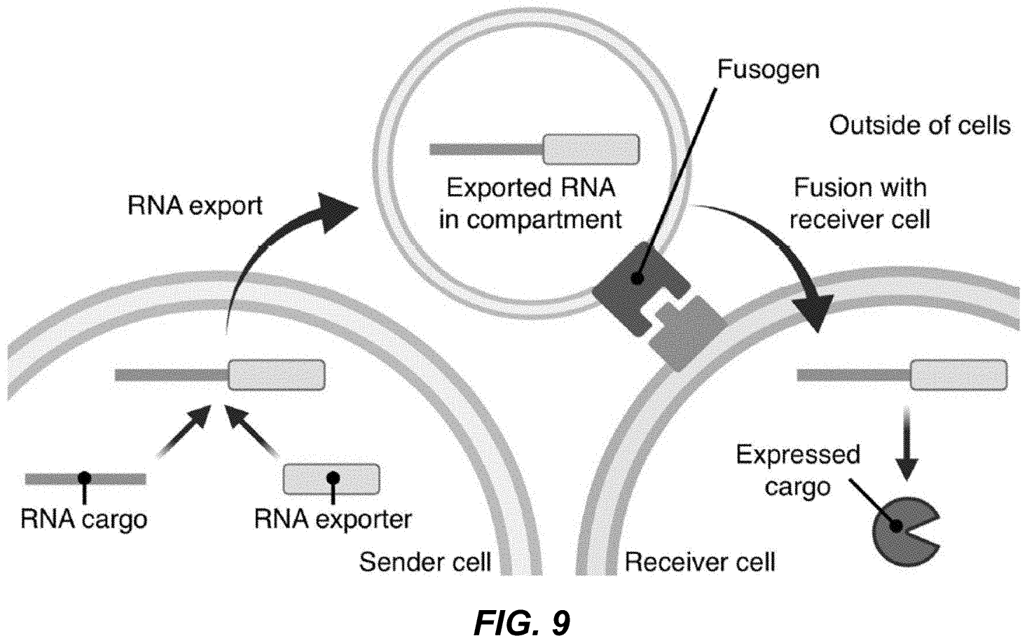

Provided herein include compositions, methods, systems, and kits for the delivery of polyribonucleotides and circuits. Disclosed herein include systems that use an engineered therapeutic sender cell to deliver a genetic program to a non-engineered receiver cell. The system can composed of several components (See, e.g., FIG. 9) such as: (1) An engineered RNA cargo, which can implement a designed molecular program both through its own molecular activity as well as through its translation as an mRNA to produce engineered protein products within the target cell. (2) An RNA exporter, which can export the RNA cargo from the sender cell in a form that can be taken up by other cells. (3) The exported RNA-containing particle can also include a cell-fusion protein (fusogen), which enables it to enter a receiver cell. (4) A control system, which can optionally enable controlled production and export of the RNA. With two or more of these components, the system as a whole can enable an engineered sender cell to controllably produce RNA cargo in a format that can be taken up and expressed in non-engineered receiver cells.

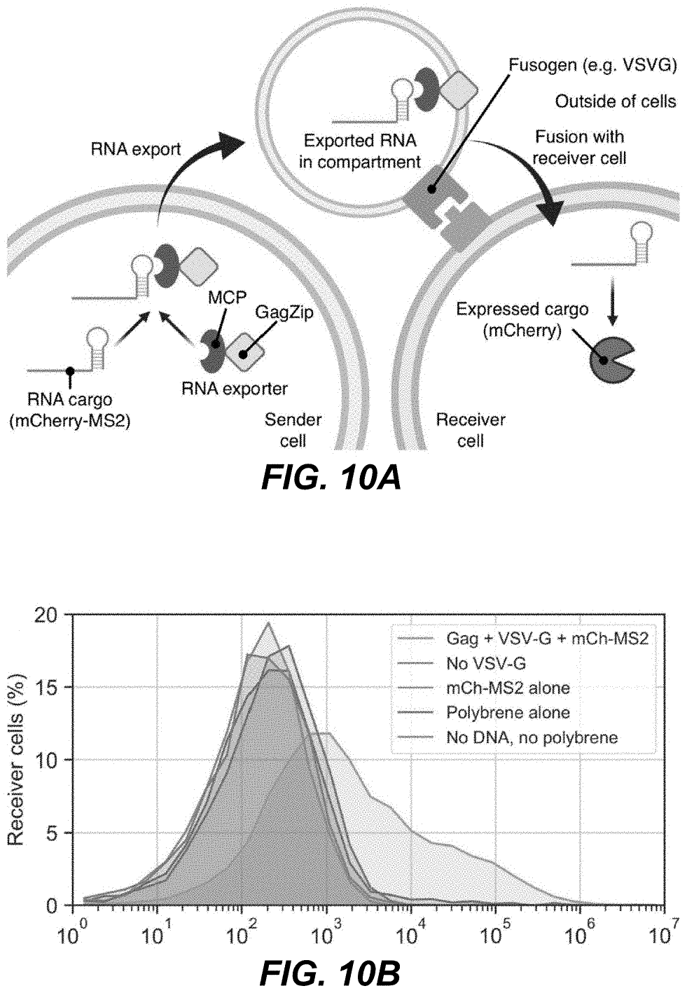

The methods, compositions, and systems provided herein can be implemented using RNA export components of viral origin, which package and secrete RNA in compartments such as virus-like particles. RNA cargo can be specifically targeted for export using RNA aptamers, cognate RNA binding proteins, and fusions thereof to form RNA exporters, such as by tagging RNA cargo with MS2 aptamers and expressing a fusion of the HIV capsid protein Gag with the aptamer-binding MS2 coat protein. The fusogen can consist of viral glycoproteins, such as the Vesicular stomatitis virus G protein. It was demonstrated in the Examples that such a system delivers RNA encoding fluorescent protein reporters from engineered HEK293T sender cells to non-engineered receiver cells (See, e.g., FIG. 10). The methods, compositions, and systems provided herein encompass a number of different embodiments and applications, and include the following:

Delivery of Genetic Circuits

Genetic circuits can enable sophisticated sensing, computation, and actuation programs. The methods, compositions, and systems provided herein enable cell-to-cell delivery of RNA circuits that perform various functions, including regulation, computation, signal processing, and control of cellular behaviors, and may be implemented by RNA or protein components, such as proteases, kinases, enzymes, etc. The methods, compositions, and systems provided herein can also deliver multiple distinct RNA molecules that together encode a functional circuit.

Conditional Activation of Sending

Controllable delivery of RNA cargo (e.g., cargo RNA molecules) to specific cells and sites within the body could improve the safety and efficacy of gene therapy, and enable new therapeutic strategies. The methods, compositions, and systems provided herein include the activation or modulation of RNA sending based on sensing of the local environment of the sender cell, including cell-surface or soluble molecules, extracellular structures, physical or chemical properties, or combinations thereof. Sending can also be controlled remotely by light, heat, ultrasound, or small molecule inducers or drugs.

Cell Targeting

The ability to achieve conditional fusion of RNA-containing compartments with target receiver cell types would allow programmable cell type specificity in the activity of the delivered RNA. The methods, compositions, and systems provided herein include the use of surface modifications of the exported RNA-containing compartments to control delivery of the payload based on features of receiver cells and interactions between the compartment and receiver cells. Such modifications can include natural or engineered viral fusogens, antibodies, ligands, or de novo designed proteins.

Conditional Circuit Function within Receiver Cells

As an additional level of control over activity of the cargo (e.g., cargo RNA molecule(s)), disclosed herein include systems for controlling expression of cargo in the receiver cell based on intracellular sensing, signal processing, or computation. This can be implemented by RNA or protein components, such as proteases, kinases, RNA-binding proteins, aptamers, microRNAs, and so forth.

Prevention of Cargo Expression in Sender Cells

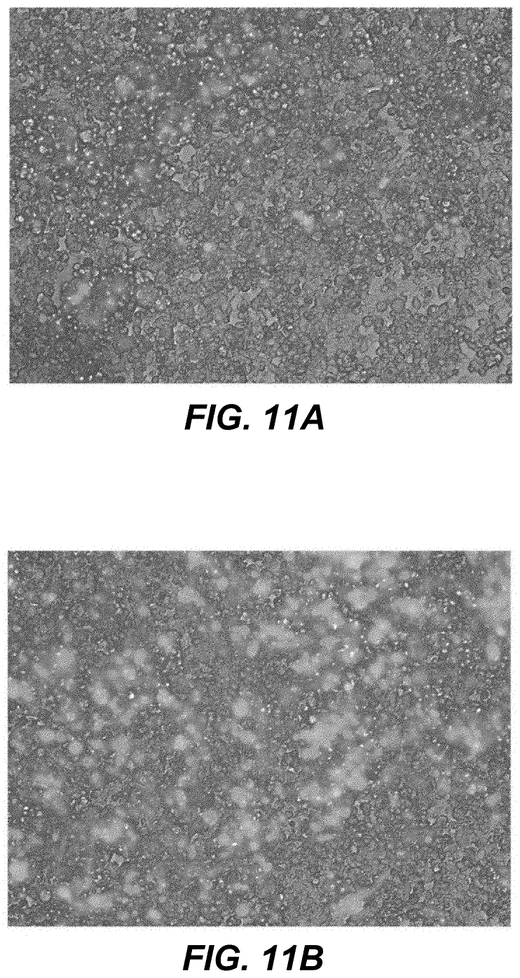

In some embodiments, activity of the RNA cargo in the sender cell could alter or impair the cell's function. Provided herein include systems for preventing activity of the cargo in the sender cell based on the cell's distinctive features. For example, the sender cell can be engineered to express a protease, which triggers degradation of the protein products of the RNA cargo, thereby preventing activity in the sender cell. The cargo can, however, be active in the non-engineered receiver cell because it lacks the protease (See, e.g., FIG. 11).

Cancer Killing

Selective killing and control of cancer cells remains challenging. Provided herein include engineered sender cells that home to tumors, activate RNA export in the presence of cancer cells, and selectively deliver cargo to cancer cells. Also provided herein are methods, compositions, and systems for the delivery of circuits that selectively kill or manipulate the state of cancer cells, such as by sensing the intracellular state of the cell and classifying it as tumor or normal based on the levels or activities of relevant molecules or pathways.

Using Delivery to Reprogram Target Cell Types In Vivo

Control over cell types and states within the body using the methods, compositions, and systems provided herein can be leveraged to address many diseases. For example, type 1 diabetes can be treated by reprogramming other pancreatic cell types into beta cells in their native context within the pancreas, circumventing issues of engraftment or allogenic origin, which in turn can be achieved by expression of transcription factors. Provided herein include systems for controlling cell type and state in receiver cells by delivering circuits, including transcription factors and epigenetic modifiers. Receiver cells can also be induced to express signals that control bystander cells (which have not received RNA cargo), such as cytokines, morphogens, ligands, or cell-surface molecules.

Genome Editing

Genomic variants cause many diseases and can be repaired using genome editors. However, efficient and specific editing is challenging, owing to the large size of the editors, and difficulty limiting their activity to defined cell types and reducing off-target edits, which tend to occur due to prolonged editing after the on-target edit has occurred. Provided herein include systems for delivery of genome editors and circuits to control their activity. Disclosed systems can achieve specific delivery and editing using the conditional activation strategies described herein. The system can enable transient editor activity via a pulse of RNA sending, or feedback control based on sensing of the corrected gene product.

Detection of Cell-Cell Proximity

Provided herein include systems for determining the relative positions of cells and probing molecular transport dynamics within tissue via delivery of sequence barcodes from cell to cell. The system can include an RNA sequence barcode, which uniquely identifies a single cell, and is delivered from sender to receiver cells. Collection and analysis of barcodes within a receiver cell, such as by sequencing or in situ hybridization, can enable determination of the abundances of barcodes from different sender cells, which in turn can be used to compute the relative distances and transport rates between pairs of cells, enabling determination of a three-dimensional physical map of the cell population or tissue. The systems, methods, compositions, and kits provided herein can, in some embodiments, be employed in concert with the systems, methods, compositions, and kits described in U.S. Provisional Patent Application No. 63/396,537, entitled, “Regenerative Editing For Molecular Recording,” filed Aug. 9, 2022, the content of which is incorporated herein by reference in its entirety.

Minimally Immunogenic Components

Therapeutic systems in organisms must evade immune recognition and rejection. The methods, compositions, and systems provided herein can use minimally immunogenic components, including RNA export and fusion systems derived from commensal viruses, endogenous viruses, or de novo designed proteins, and humanized components such as receptors, fusogens, or cargo proteins. The methods, compositions, and systems provided herein can use components to actively suppress immune rejection, such as expression or downregulation of immune modulators (e.g. CD47, B2M, MHC) on the sender or receiver cells, or extracellular compartments.

Disclosed herein include compositions. In some embodiments, the composition is or comprises a nucleic acid composition. The nucleic acid composition can comprise: one or more first polynucleotide(s) encoding an RNA exporter protein; one or more second polynucleotide(s) each encoding one or more cargo RNA molecule(s); and/or one or more third polynucleotide(s) encoding a fusogen. In some embodiments, the RNA exporter protein comprises: an RNA-binding domain, a membrane-binding domain, and an interaction domain capable of nucleating self-assembly. In some embodiments, a plurality of RNA exporter proteins are capable of self-assembling into lipid-enveloped nanoparticles (LNs) secreted from a sender cell in which the RNA exporter proteins are expressed, thereby generating a population of LNs comprising the fusogen and exported cargo RNA molecule(s).

Disclosed herein include compositions. In some embodiments, the composition is or comprises a population of sender cells. In some embodiments, the composition comprises: a population of sender cells comprising a nucleic acid composition disclosed herein. The population of sender cells can comprise: one or more first polynucleotide(s) encoding an RNA exporter protein; one or more second polynucleotide(s) each encoding one or more cargo RNA molecule(s); and/or one or more third polynucleotide(s) encoding a fusogen. In some embodiments, the RNA exporter protein comprises: an RNA-binding domain, a membrane-binding domain, and an interaction domain capable of nucleating self-assembly. In some embodiments, a plurality of RNA exporter proteins are capable of self-assembling into lipid-enveloped nanoparticles (LNs) secreted from a sender cell in which the RNA exporter proteins are expressed, thereby generating a population of LNs comprising the fusogen and exported cargo RNA molecule(s).

The population of LNs can be capable of fusing with receiver cells, thereby delivering the cargo RNA molecule(s) to said receiver cells. The fusogen can be capable of mediating the fusion of the lipid envelope of the LN and a lipid bilayer of a receiver cell. The fusogen can comprise or can be derived from a SNARE protein, dynamin, an FF protein, a FAST protein, a viral fusogenic glycoprotein, or any combination thereof. The viral fusogenic glycoprotein can be a glycoprotein from retroviridae, herpesviridae, poxviridae, hepadnaviridae, flaviviridae, togavoridae, coronaviridae, hepatitis D virus, orthomyxoviridae, paramyxoviridae, filoviridae, rhabdoviridae, bunyaviridae, orthopoxivridae, or any combination thereof. The viral fusogenic glycoprotein can be selected from the group comprising HBsAg of HBV (e.g., M-, S- or L-HBsAg), E1 and/or E2 proteins of HCV, HA (hemaglutinin) and NA (neuraminidase) of Influenza, glycoprotein G of VSV, glycoprotein GP of Ebola or Marburg virus, glycoproteins Gp120 (or its CD4-binding domain) and Gp41 of lentiviruses, envelop protein (DENV E) and pre-membrane protein (prM DENV) of Dengue virus, two envelope glycoproteins of Hantaan virus, glycoprotein E2 of Chikungunya virus, glycoproteins HN and F of Newcastle virus, gp85 and gp37 of Rous sarcoma virus, and protein E and prM of Murray Valley encephalitis virus. In some embodiments, the cargo RNA molecule(s) comprise packing signal(s).

In some embodiments, the RNA exporter protein is: a chimeric fusion protein; or a multi-subunit protein. The RNA exporter protein can comprise two or more components configured to dimerize via dimerization domain(s). The RNA binding domain can be capable of binding the packing signal(s), and the cargo RNA molecule(s) can be specifically packaged into the LNs via interaction of the packing signal(s) with the RNA-binding domain of the RNA exporter protein. The abundance of cargo RNA molecule(s) exported to the exterior of a sender cell can be at least about 1.1-fold (e.g., 0.1-fold, 1.2-fold, 1.3-fold, 1.4-fold, 1.5-fold, 1.6-fold, 1.7-fold, 1.8-fold, 1.8-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) higher as compared to a sender cell wherein (i) the packing signal(s) are absent from the cargo RNA molecule(s) and/or (ii) the RNA exporter protein does not comprise an RNA binding domain. The packing signal(s) can comprise an array of at least about 2, 3, 4, 5, 6, 7, 8, 9, 10, 11, 12, 13, or 14, tandem repeats of an aptamer (e.g., a MS2 stem-loop aptamer). The rate of export of cargo RNA molecule(s) can be capable of being configured by varying the number of tandem repeats.

The RNA exporter protein can be configured to be minimally perturbative to cellular physiology and/or minimally immunogenic. The RNA exporter protein can comprise or can be derived from one or more components of viral origin, one or more components of a non-viral compartmentalization and secretion system, and/or one or more components of de novo designed proteins. The RNA exporter protein can comprise a capsid protein of viral origin, optionally fused with an RNA binding protein. The one or more first polynucleotide(s) encoding the RNA exporter protein can comprise packing signal(s), and the LNs thereby can comprise RNA molecules encoding the RNA exporter protein. The RNA binding domain can comprise or can be derived from an RNA binding protein. The packing signal(s) can comprise a Ku binding hairpin and the RNA binding protein can be Ku. The packing signal(s) can comprise a telomerase Sm7 binding motif and the RNA binding protein can be Sm7. The packing signal(s) can comprise an MS2 phage operator stem-loop and the RNA binding protein can be MS2 Coat Protein (MCP). The packing signal(s) can comprise a PP7 phage operator stem-loop and the RNA binding protein can be PP7 Coat Protein (PCP). The packing signal(s) can comprise an SfMu phage Com stem-loop and the RNA binding protein can be Com RNA binding protein. The packing signal(s) can comprise a PUF binding site (PBS) and the RNA binding protein can be Pumilio/fem-3 mRNA binding factor (PUF). The packing signal(s) can comprise an MMLV packing signal (Psi) and the RNA binding protein can be MMLV. The RNA binding domain can comprise or can be derived from a catalytically inactivated programmable nuclease configured to bind the packing signal(s) (e.g., catalytically inactivated CRISPR-Cas9 or -Cas13). The RNA exporter protein can comprise at least a portion of a viral capsid protein (e.g., a retroviral Gag protein) and in some embodiments at least a portion of the nucleocapsid domain, matrix domain, a zinc finger domain, a p1 domain, capsid domain and/or a p1-p6 domain can be removed from said viral capsid protein. In some embodiments, the nucleocapsid domain is replaced with a leucine zipper (e.g., the leucine zipper of GCN4). The interaction domain can comprise or can be derived from a viral capsid protein (e.g., a retroviral Gag protein).

The interaction domain can comprise dimerization domain(s) (e.g., a leucine zipper dimerization domain from GCN4 (Zip)). The dimerization domain(s) can comprise or can be derived from SYNZIP1, SYNZIP2, SYNZIP3, SYNZIP4, SYNZIP5, SYNZIP6, SYNZIP7, SYNZIP8, SYNZIP9, SYNZIP10, SYNZIP11, SYNZIP12, SYNZIP13, SYNZIP14, SYNZIP15, SYNZIP16, SYNZIP17, SYNZIP18, SYNZIP19, SYNZIP20, SYNZIP21, SYNZIP22, SYNZIP23, BATF, FOS, ATF4, BACH1, JUND, NFE2L3, AZip, BZip, a PDZ domain ligand, an SH3 domain, a PDZ domain, a GTPase binding domain, a leucine zipper domain, an SH2 domain, a PTB domain, an FHA domain, a WW domain, a 14-3-3 domain, a death domain, a caspase recruitment domain, a bromodomain, a chromatin organization modifier, a shadow chromo domain, an F-box domain, a HECT domain, a RING finger domain, a sterile alpha motif domain, a glycine-tyrosine-phenylalanine domain, a SNAP domain, a VHS domain, an ANK repeat, an armadillo repeat, a WD40 repeat, an MH2 domain, a calponin homology domain, a Dbl homology domain, a gelsolin homology domain, a PB1 domain, a SOCS box, an RGS domain, a Toll/IL-1 receptor domain, a tetratricopeptide repeat, a TRAF domain, a Bcl-2 homology domain, a coiled-coil domain, a bZIP domain, portions thereof, variants thereof, or any combination thereof. The dimerization domain(s) can be a homodimerization domain or a multimerization domain, such as, for example, a homo- or hetero-dimerizing or multimerizing leucine zipper, a PDZ domains, a SH3 domain, aGBD domain, or any combination thereof.

In some embodiments, the RNA exporter protein self-assembles to form nanocages, and wherein the LNs can comprise a plurality of said nanocages. The interaction domain can comprise I3-01 and/or the membrane binding domain can comprise rat phospholipase C delta Pleckstrin Homology domain. The RNA exporter protein can comprise enveloped protein nanocage domain EPN24 fused with an RNA binding protein. In some embodiments, the RNA exporter protein comprises: a myristoylation motif of HIV-1 NL4-3 Gag, optionally amino acid residues 2-6; a myristoylation/palmitoylation motif of Lyn kinase, optionally amino acid residues 2-13; a Pleckstrin Homology domain of rat phospholipase Cδ; and/or a p6 domain of HIV-1 NL4-3 Gag. The RNA exporter protein can be configured to operate in a cell type and/or species different than the cell type and/or species in which at least one parental component of said RNA exporter protein operates. The RNA exporter protein can be a species-chimeric RNA exporter protein. The RNA exporter protein can comprise a chimeric viral capsid protein wherein the matrix domain is replaced with the matrix domain of another virus. The RNA exporter protein can comprise an amino acid sequence at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values, identical to any one of SEQ ID NOS: 1-24 (Table 1). In some embodiments, the expression of the RNA exporter protein in the sender cell(s) does not alter the endogenous transcriptome, morphology, and/or physiology of said sender cell(s). Table 2 shows exemplary implementations of RNA exporters.

TABLE 1
AMINO ACID SEQUENCES OF RNA EXPORTER PROTEINS

| | SEQ | |
|---|---|---|
| Name | ID NO | Sequence |
| EPN1 | 1 | MGARASGSKSGSGSDSGSKIEELFKKHKIVAVLRANSVEEAKKKALAVFLGGVH |
| | | LIEITFTVPDADTVIKELSFLKEMGAIIGAGTVTSVEQCRKAVESGAEFIVSPH |
| | | LDEEISQFCKEKGVFYMPGVMTPTELVKAMKLGHTILKLFPGEVVGPQFVKAMK |
| | | GPFPNVKFVPTGGVNLDNVCEWFKAGVLAVGVGSALVKGTPVEVAEKAKAFVEK |
| | | IRGCTEQKLISEEDLQSRPEPTAPPEESFRSGVETTTPPQKQEPIDKELYPLTS |
| | | LRSLFGNDPSSQ |
| EPN1-MCP | 2 | MGARASGSKSGSGSDSGSKIEELFKKHKIVAVLRANSVEEAKKKALAVFLGGVH |
| | | LIEITFTVPDADTVIKELSFLKEMGAIIGAGTVTSVEQCRKAVESGAEFIVSPH |
| | | LDEEISQFCKEKGVFYMPGVMTPTELVKAMKLGHTILKLFPGEVVGPQFVKAMK |
| | | GPFPNVKFVPTGGVNLDNVCEWFKAGVLAVGVGSALVKGTPVEVAEKAKAFVEK |
| | | IRGCTEQKLISEEDLQSRPEPTAPPEESFRSGVETTTPPQKQEPIDKELYPLTS |
| | | LRSLFGNDPSSQASNFTQFVLVDNGGTGDVTVAPSNFANGVAEWISSNSRSQAY |
| | | KVTCSVRQSSAQKRKYTIKVEVPKVATQTVGGVELPVAAWRSYLNMELTIPIFA |
| | | TNSDCELIVKAMQGLLKDGNPIPSAIAANSGIY |
| EPN1-MCP_I | 3 | MGARASGSKSGSGSDSGSKIEELFKKHKIVAVLRANSVEEAKKKALAVFLGGVH |
| | | LIEITFTVPDADTVIKELSFLKEMGAIIGAGTVTSVEQCRKAVESGAEFIVSPH |
| | | LDEEISQFCKEKGVFYMPGVMTPTELVKAMKLGHTILKLFPGEVVGPQFVKAMK |
| | | GPFPNVKFVPTGGVNLDNVCEWFKAGVLAVGVGSALVKGTPVEVAEKAKAFVEK |
| | | IRGCTASNFTQFVLVDNGGTGDVTVAPSNFANGVAEWISSNSRSQAYKVTCSVR |
| | | QSSAQKRKYTIKVEVPKVATQTVGGVELPVAAWRSYLNMELTIPIFATNSDCEL |
| | | IVKAMQGLLKDGNPIPSAIAANSGIYEQKLISEEDLQSRPEPTAPPEESFRSGV |
| | | ETTTPPQKQEPIDKELYPLTSLRSLFGNDPSSQ |
| EPN1-MCPMyc | 4 | MGARASGSKSGSGSDSGSKIEELFKKHKIVAVLRANSVEEAKKKALAVFLGGVH |
| | | LIEITFTVPDADTVIKELSFLKEMGAIIGAGTVTSVEQCRKAVESGAEFIVSPH |
| | | LDEEISQFCKEKGVFYMPGVMTPTELVKAMKLGHTILKLFPGEVVGPQFVKAMK |
| | | GPFPNVKFVPTGGVNLDNVCEWFKAGVLAVGVGSALVKGTPVEVAEKAKAFVEK |
| | | IRGCTEQKLISEEDLASNFTQFVLVDNGGTGDVTVAPSNFANGVAEWISSNSRS |
| | | QAYKVTCSVRQSSAQKRKYTIKVEVPKVATQTVGGVELPVAAWRSYLNMELTIP |
| | | IFATNSDCELIVKAMQGLLKDGNPIPSAIAANSGIYLQSRPEPTAPPEESFRSG |
| | | VETTTPPQKQEPIDKELYPLTSLRSLFGNDPSSQ |
| EPN11 | 5 | MGCIKSKRKDNLNLQSRPEPTAPPEESFRSGVETTTPPQKQEPIDKELYPLTSL |
| | | RSLFGNDPSSQKIEELFKKHKIVAVLRANSVEEAKKKALAVFLGGVHLIEITFT |
| | | VPDADTVIKELSFLKEMGAIIGAGTVTSVEQCRKAVESGAEFIVSPHLDEEISQ |
| | | FCKEKGVFYMPGVMTPTELVKAMKLGHTILKLFPGEVVGPQFVKAMKGPFPNVK |
| | | FVPTGGVNLDNVCEWFKAGVLAVGVGSALVKGTPVEVAEKAKAFVEKIRGCTEQ |
| | | KLISEEDL |
| EPN11-MCP | 6 | MGCIKSKRKDNLNLQSRPEPTAPPEESFRSGVETTTPPQKQEPIDKELYPLTSL |
| | | RSLFGNDPSSQKIEELFKKHKIVAVLRANSVEEAKKKALAVFLGGVHLIEITFT |
| | | VPDADTVIKELSFLKEMGAIIGAGTVTSVEQCRKAVESGAEFIVSPHLDEEISQ |
| | | FCKEKGVFYMPGVMTPTELVKAMKLGHTILKLFPGEVVGPQFVKAMKGPFPNVK |
| | | FVPTGGVNLDNVCEWFKAGVLAVGVGSALVKGTPVEVAEKAKAFVEKIRGCTEQ |
| | | KLISEEDLASNFTQFVLVDNGGTGDVTVAPSNFANGVAEWISSNSRSQAYKVTC |
| | | SVRQSSAQKRKYTIKVEVPKVATQTVGGVELPVAAWRSYLNMELTIPIFATNSD |
| | | CELIVKAMQGLLKDGNPIPSAIAANSGIY |
| EPN11-MCP_I | 7 | MGCIKSKRKDNLNLQSRPEPTAPPEESFRSGVETTTPPQKQEPIDKELYPLTSL |
| | | RSLFGNDPSSQKIEELFKKHKIVAVLRANSVEEAKKKALAVFLGGVHLIEITFT |
| | | VPDADTVIKELSFLKEMGAIIGAGTVTSVEQCRKAVESGAEFIVSPHLDEEISQ |
| | | FCKEKGVFYMPGVMTPTELVKAMKLGHTILKLFPGEVVGPQFVKAMKGPFPNVK |
| | | FVPTGGVNLDNVCEWFKAGVLAVGVGSALVKGTPVEVAEKAKAFVEKIRGCTAS |
| | | NFTQFVLVDNGGTGDVTVAPSNFANGVAEWISSNSRSQAYKVTCSVRQSSAQKR |
| | | KYTIKVEVPKVATQTVGGVELPVAAWRSYLNMELTIPIFATNSDCELIVKAMQG |
| | | LLKDGNPIPSAIAANSGIYEQKLISEEDL |
| EPN11-MCP_p6 | 8 | MGCIKSKRKDNLNLQSRPEPTAPPEESFRSGVETTTPPQKQEPIDKELYPLTSL |
| | | RSLFGNDPSSQASNFTQFVLVDNGGTGDVTVAPSNFANGVAEWISSNSRSQAYK |
| | | VTCSVRQSSAQKRKYTIKVEVPKVATQTVGGVELPVAAWRSYLNMELTIPIFAT |
| | | NSDCELIVKAMQGLLKDGNPIPSAIAANSGIYKIEELFKKHKIVAVLRANSVEE |
| | | AKKKALAVFLGGVHLIEITFTVPDADTVIKELSFLKEMGAIIGAGTVTSVEQCR |
| | | KAVESGAEFIVSPHLDEEISQFCKEKGVFYMPGVMTPTELVKAMKLGHTILKLF |
| | | PGEVVGPQFVKAMKGPFPNVKFVPTGGVNLDNVCEWFKAGVLAVGVGSALVKGT |
| | | PVEVAEKAKAFVEKIRGCTEQKLISEEDL |
| EPN24 | 9 | MHGLQDDPDLQALLKGSQLLKVKSSSWRRERFYKLQEDCKTIWQESRKVMRSPE |
| | | SQLFSIEDIQEVRMGHRTEGLEKFARDIPEDRCFSIVFKDQRNTLDLIAPSPAD |
| | | AQHWVQGLRKIIHHSGSMDQRQKLQSRPEPTAPPEESFRSGVETTTPPQKQEPI |
| | | DKELYPLTSLRSLFGNDPSSQKIEELFKKHKIVAVLRANSVEEAKKKALAVFLG |
| | | GVHLIEITFTVPDADTVIKELSFLKEMGAIIGAGTVTSVEQCRKAVESGAEFIV |
| | | SPHLDEEISQFCKEKGVFYMPGVMTPTELVKAMKLGHTILKLFPGEVVGPQFVK |
| | | AMKGPFPNVKFVPTGGVNLDNVCEWFKAGVLAVGVGSALVKGTPVEVAEKAKAF |
| | | VEKIRGCTEQKLISEEDL |
| EPN24-MCP | 10 | MHGLQDDPDLQALLKGSQLLKVKSSSWRRERFYKLQEDCKTIWQESRKVMRSPE |
| | | SQLFSIEDIQEVRMGHRTEGLEKFARDIPEDRCFSIVFKDQRNTLDLIAPSPAD |
| | | AQHWVQGLRKIIHHSGSMDQRQKLQSRPEPTAPPEESFRSGVETTTPPQKQEPI |
| | | DKELYPLTSLRSLFGNDPSSQKIEELFKKHKIVAVLRANSVEEAKKKALAVFLG |
| | | GVHLIEITFTVPDADTVIKELSFLKEMGAIIGAGTVTSVEQCRKAVESGAEFIV |
| | | SPHLDEEISQFCKEKGVFYMPGVMTPTELVKAMKLGHTILKLFPGEVVGPQFVK |
| | | AMKGPFPNVKFVPTGGVNLDNVCEWFKAGVLAVGVGSALVKGTPVEVAEKAKAF |
| | | VEKIRGCTEQKLISEEDLASNFTQFVLVDNGGTGDVTVAPSNFANGVAEWISSN |
| | | SRSQAYKVTCSVRQSSAQKRKYTIKVEVPKVATQTVGGVELPVAAWRSYLNMEL |
| | | TIPIFATNSDCELIVKAMQGLLKDGNPIPSAIAANSGIY |
| EPN24-MCP_I | 11 | MHGLQDDPDLQALLKGSQLLKVKSSSWRRERFYKLQEDCKTIWQESRKVMRSPE |
| | | SQLFSIEDIQEVRMGHRTEGLEKFARDIPEDRCFSIVFKDQRNTLDLIAPSPAD |
| | | AQHWVQGLRKIIHHSGSMDQRQKLQSRPEPTAPPEESFRSGVETTTPPQKQEPI |
| | | DKELYPLTSLRSLFGNDPSSQKIEELFKKHKIVAVLRANSVEEAKKKALAVFLG |
| | | GVHLIEITFTVPDADTVIKELSFLKEMGAIIGAGTVTSVEQCRKAVESGAEFIV |
| | | SPHLDEEISQFCKEKGVFYMPGVMTPTELVKAMKLGHTILKLFPGEVVGPQFVK |
| | | AMKGPFPNVKFVPTGGVNLDNVCEWFKAGVLAVGVGSALVKGTPVEVAEKAKAF |
| | | VEKIRGCTASNFTQFVLVDNGGTGDVTVAPSNFANGVAEWISSNSRSQAYKVTC |
| | | SVRQSSAQKRKYTIKVEVPKVATQTVGGVELPVAAWRSYLNMELTIPIFATNSD |
| | | CELIVKAMQGLLKDGNPIPSAIAANSGIYEQKLISEEDL |
| EPN24-MCP_p6 | 12 | MHGLQDDPDLQALLKGSQLLKVKSSSWRRERFYKLQEDCKTIWQESRKVMRSPE |
| | | SQLFSIEDIQEVRMGHRTEGLEKFARDIPEDRCFSIVFKDQRNTLDLIAPSPAD |
| | | AQHWVQGLRKIIHHSGSMDQRQKLQSRPEPTAPPEESFRSGVETTTPPQKQEPI |
| | | DKELYPLTSLRSLFGNDPSSQASNFTQFVLVDNGGTGDVTVAPSNFANGVAEWI |
| | | SSNSRSQAYKVTCSVRQSSAQKRKYTIKVEVPKVATQTVGGVELPVAAWRSYLN |
| | | MELTIPIFATNSDCELIVKAMQGLLKDGNPIPSAIAANSGIYKIEELFKKHKIV |
| | | AVLRANSVEEAKKKALAVFLGGVHLIEITFTVPDADTVIKELSFLKEMGAIIGA |
| | | GTVTSVEQCRKAVESGAEFIVSPHLDEEISQFCKEKGVFYMPGVMTPTELVKAM |
| | | KLGHTILKLFPGEVVGPQFVKAMKGPFPNVKFVPTGGVNLDNVCEWFKAGVLAV |
| | | GVGSALVKGTPVEVAEKAKAFVEKIRGCTEQKLISEEDL |
| Gag (wild-type | 13 | MGARASVLSGGELDRWEKIRLRPGGKKKYKLKHIVWASRELERFAVNPGLLETS |
| HIV) | | EGCRQILGQLQPSLQTGSEELRSLYNTVATLYCVHQRIEIKDTKEALDKIEEEQ |
| | | NKSKKKAQQAAADTGHSNQVSQNYPIVQNIQGQMVHQAISPRTLNAWVKVVEEK |
| | | AFSPEVIPMFSALSEGATPQDLNTMLNTVGGHQAAMQMLKETINEEAAEWDRVH |
| | | PVHAGPIAPGQMREPRGSDIAGTTSTLQEQIGWMTNNPPIPVGEIYKRWIILGL |
| | | NKIVRMYSPTSILDIRQGPKEPFRDYVDRFYKTLRAEQASQEVKNWMTETLLVQ |
| | | NANPDCKTILKALGPAATLEEMMTACQGVGGPGHKARVLAEAMSQVTNSATIMM |
| | | QRGNFRNQRKIVKCFNCGKEGHTARNCRAPRKKGCWKCGKEGHQMKDCTERQAN |
| | | FLGKIWPSYKGRPGNFLQSRPEPTAPPEESFRSGVETTTPPQKQEPIDKELYPL |
| | | TSLRSLFGNDPSSQ |
| Gag (wild-type | 14 | MGQTVTTPLSLTLGHWKDVERIAHNQSVDVKKRRWVTFCSAEWPTFNVGWPRDG |
| MMLV) | | TFNRDLITQVKIKVFSPGPHGHPDQVPYIVTWEALAFDPPPWVKPFVHPKPPPP |
| | | LPPSAPSLPLEPPRSTPPRSSLYPALTPSLGAKPKPQVLSDSGGPLIDLLTEDP |
| | | PPYRDPRPPPSDRDGNGGEATPAGEAPDPSPMASRLRGRREPPVADSTTSQAFP |
| | | LRAGGNGQLQYWPFSSSDLYNWKNNNPSFSEDPGKLTALIESVLITHQPTWDDC |
| | | QQLLGTLLTGEEKQRVLLEARKAVRGDDGRPTQLPNEVDAAFPLERPDWDYTTQ |
| | | AGRNHLVHYRQLLLAGLQNAGRSPTNLAKVKGITQGPNESPSAFLERLKEAYRR |
| | | YTPYDPEDPGQETNVSMSFIWQSAPDIGRKLERLEDLKNKTLGDLVREAEKIFN |
| | | KRETPEEREERIRRETEEKEERRRTEDEQKEKERDRRRHREMSKLLATVVSGQK |
| | | QDRQGGERRRSQLDRDQCAYCKEKGHWAKDCPKKPRGPRGPRPQTSLL |
| Gag_MHIV_MA- | 15 | MGQTVTTPLSLTLGHWKDVERIAHNQSVDVKKRRWVTFCSAEWPTFNVGWPRDG |
| MCP | | TFNRDLITQVKIKVFSPGPHGHPDQVPYIVTWEALAFDPPPWVKPFVHPKPPPP |
| | | LPPSAPSLPLEPPRSTPPRSSLYPIVQNIQGQMVHQAISPRTLNAWVKVVEEKA |
| | | FSPEVIPMFSALSEGATPQDLNTMLNTVGGHQAAMQMLKETINEEAAEWDRVHP |
| | | VHAGPIAPGQMREPRGSDIAGTTSTLQEQIGWMTNNPPIPVGEIYKRWIILGLN |
| | | KIVRMYSPTSILDIRQGPKEPFRDYVDRFYKTLRAEQASQEVKNWMTETLLVQN |
| | | ANPDCKTILKALGPAATLEEMMTACQGVGGPGHKARVLAEAMSQVTNSATIMMQ |
| | | RGNFRNQRKIVKCFNCGKEGHTARNCRAPRKKGCWKCGKEGHQMKDCTERQANF |
| | | LGKIWPSYKGRPGNFLQSRPEPTAPPEESFRSGVETTTPPQKQEPIDKELYPLT |
| | | SLRSLFGNDPSSQASNFTQFVLVDNGGTGDVTVAPSNFANGVAEWISSNSRSQA |
| | | YKVTCSVRQSSAQKRKYTIKVEVPKVATQTVGGVELPVAAWRSYLNMELTIPIF |
| | | ATNSDCELIVKAMQGLLKDGNPIPSAIAANSGIY |
| Gag-MCP | 16 | MGARASVLSGGELDRWEKIRLRPGGKKKYKLKHIVWASRELERFAVNPGLLETS |
| | | EGCRQILGQLQPSLQTGSEELRSLYNTVATLYCVHQRIEIKDTKEALDKIEEEQ |
| | | NKSKKKAQQAAADTGHSNQVSQNYPIVQNIQGQMVHQAISPRTLNAWVKVVEEK |
| | | AFSPEVIPMFSALSEGATPQDLNTMLNTVGGHQAAMQMLKETINEEAAEWDRVH |
| | | PVHAGPIAPGQMREPRGSDIAGTTSTLQEQIGWMTNNPPIPVGEIYKRWIILGL |
| | | NKIVRMYSPTSILDIRQGPKEPFRDYVDRFYKTLRAEQASQEVKNWMTETLLVQ |
| | | NANPDCKTILKALGPAATLEEMMTACQGVGGPGHKARVLAEAMSQVTNSATIMM |
| | | QRGNFRNQRKIVKCFNCGKEGHTARNCRAPRKKGCWKCGKEGHQMKDCTERQAN |
| | | FLGKIWPSYKGRPGNFLQSRPEPTAPPEESFRSGVETTTPPQKQEPIDKELYPL |
| | | TSLRSLFGNDPSSQASNFTQFVLVDNGGTGDVTVAPSNFANGVAEWISSNSRSQ |
| | | AYKVTCSVRQSSAQKRKYTIKVEVPKVATQTVGGVELPVAAWRSYLNMELTIPI |
| | | FATNSDCELIVKAMQGLLKDGNPIPSAIAANSGIY |
| Gag-MCPZF2 | 17 | MGARASVLSGGELDRWEKIRLRPGGKKKYKLKHIVWASRELERFAVNPGLLETS |
| | | EGCRQILGQLQPSLQTGSEELRSLYNTVATLYCVHQRIEIKDTKEALDKIEEEQ |
| | | NKSKKKAQQAAADTGHSNQVSQNYPIVQNIQGQMVHQAISPRTLNAWVKVVEEK |
| | | AFSPEVIPMFSALSEGATPQDLNTMLNTVGGHQAAMQMLKETINEEAAEWDRVH |
| | | PVHAGPIAPGQMREPRGSDIAGTTSTLQEQIGWMTNNPPIPVGEIYKRWIILGL |
| | | NKIVRMYSPTSILDIRQGPKEPFRDYVDRFYKTLRAEQASQEVKNWMTETLLVQ |
| | | NANPDCKTILKALGPAATLEEMMTACQGVGGPGHKARVLAEAMSQVTNSATIMM |
| | | QRGNFRNQRKIVKCFNCGKEGHTARNCRAPRKKGCWKCGKEGHQMKDCTEASNF |
| | | TQFVLVDNGGTGDVTVAPSNFANGVAEWISSNSRSQAYKVTCSVRQSSAQKRKY |
| | | TIKVEVPKVATQTVGGVELPVAAWRSYLNMELTIPIFATNSDCELIVKAMQGLL |
| | | KDGNPIPSAIAANSGIYRQANFLGKIWPSYKGRPGNFLQSRPEPTAPPEESFRS |
| | | GVETTTPPQKQEPIDKELYPLTSLRSLFGNDPSSQ |
| Gag-MCPΔZF2 | 18 | MGARASVLSGGELDRWEKIRLRPGGKKKYKLKHIVWASRELERFAVNPGLLETS |
| | | EGCRQILGQLQPSLQTGSEELRSLYNTVATLYCVHQRIEIKDTKEALDKIEEEQ |
| | | NKSKKKAQQAAADTGHSNQVSQNYPIVQNIQGQMVHQAISPRTLNAWVKVVEEK |
| | | AFSPEVIPMFSALSEGATPQDLNTMLNTVGGHQAAMQMLKETINEEAAEWDRVH |
| | | PVHAGPIAPGQMREPRGSDIAGTTSTLQEQIGWMTHNPPIPVGEIYKRWIILGL |
| | | NKIVRMYSPTSILDIRQGPKEPFRDYVDRFYKTLRAEQASQEVKNWMTETLLVQ |
| | | NANPDCKTILKALGPGATLEEMMTACQGVGGPGHKARVLAEAMSQVTNPATIMI |
| | | QKGNFRNQRKTVKCFNCGKEGHIAKNCRAPRKKGVASNFTQFVLVDNGGTGDVT |
| | | VAPSNFANGVAEWISSNSRSQAYKVTCSVRQSSAQKRKYTIKVEVPKVATQTVG |
| | | GVELPVAAWRSYLNMELTIPIFATNSDCELIVKAMQGLLKDGNPIPSAIAANSG |
| | | IYTERQANFLGKIWPSHKGRPGNFLQSRPEPTAPPEESFRFGEETTTPSQKQEP |
| | | IDKELYPLASLRSLFGSDPSSQ |
| GagZip-MCP | 19 | MGARASVLSGGELDRWEKIRLRPGGKKKYKLKHIVWASRELERFAVNPGLLETS |
| | | EGCRQILGQLQPSLQTGSEELRSLYNTVATLYCVHQRIEIKDTKEALDKIEEEQ |
| | | NKSKKKAQQAAADTGHSNQVSQNYPIVQNIQGQMVHQAISPRTLNAWVKVVEEK |
| | | AFSPEVIPMFSALSEGATPQDLNTMLNTVGGHQAAMQMLKETINEEAAEWDRVH |
| | | PVHAGPIAPGQMREPRGSDIAGTTSTLQEQIGWMTNNPPIPVGEIYKRWIILGL |
| | | NKIVRMYSPTSILDIRQGPKEPFRDYVDRFYKTLRAEQASQEVKNWMTETLLVQ |
| | | NANPDCKTILKALGPAATLEEMMTACQGVGGPGHKARVLAEAMSQVTNSATIML |
| | | QRMKQLEDKVEELLSKNYHLENEVARLKKLVGEFLGKIWPSYKGRPGNFLQSRP |
| | | EPTAPPEESFRSGVETTTPPQKQEPIDKELYPLTSLRSLFGNDPSSQASNFTQF |
| | | VLVDNGGTGDVTVAPSNFANGVAEWISSNSRSQAYKVTCSVRQSSAQKRKYTIK |
| | | VEVPKVATQTVGGVELPVAAWRSYLNMELTIPIFATNSDCELIVKAMQGLLKDG |
| | | NPIPSAIAANSGIY |
| GagZip-MCP- | 20 | MGARASVLSGGELDRWEKIRLRPGGKKKYKLKHIVWASRELERFAVNPGLLETS |
| dpol | | EGCRQILGQLQPSLQTGSEELRSLYNTVATLYCVHQRIEIKDTKEALDKIEEEQ |
| | | NKSKKKAQQAAADTGHSNQVSQNYPIVQNIQGQMVHQAISPRTLNAWVKVVEEK |
| | | AFSPEVIPMFSALSEGATPQDLNTMLNTVGGHQAAMQMLKETINEEAAEWDRVH |
| | | PVHAGPIAPGQMREPRGSDIAGTTSTLQEQIGWMTNNPPIPVGEIYKRWIILGL |
| | | NKIVRMYSPTSILDIRQGPKEPFRDYVDRFYKTLRAEQASQEVKNWMTETLLVQ |
| | | NANPDCKTILKALGPAATLEEMMTACQGVGGPGHKARVLAEAMSQVTNSATIMM |
| | | QRGNFRNQRKIVKCFNCGKEGHTARNCRAPRKKGCWKCGKEGHQMKDCTERQAN |
| | | FLGKIWPSYKGRPGNFLQSRPEPTAPPEESFRSGVETTTPPQKQEPIDKELYPL |
| | | TSLRSLFGNDPSSQASNFTQFVLVDNGGTGDVTVAPSNFANGVAEWISSNSRSQ |
| | | AYKVTCSVRQSSAQKRKYTIKVEVPKVATQTVGGVELPVAAWRSYLNMELTIPI |
| | | FATNSDCELIVKAMQGLLKDGNPIPSAIAANSGIY |
| GagZip-MCPZip | 21 | MGARASVLSGGELDRWEKIRLRPGGKKKYKLKHIVWASRELERFAVNPGLLETS |
| | | EGCRQILGQLQPSLQTGSEELRSLYNTVATLYCVHQRIEIKDTKEALDKIEEEQ |
| | | NKSKKKAQQAAADTGHSNQVSQNYPIVQNIQGQMVHQAISPRTLNAWVKVVEEK |
| | | AFSPEVIPMFSALSEGATPQDLNTMLNTVGGHQAAMQMLKETINEEAAEWDRVH |
| | | PVHAGPIAPGQMREPRGSDIAGTTSTLQEQIGWMTNNPPIPVGEIYKRWIILGL |
| | | NKIVRMYSPTSILDIRQGPKEPFRDYVDRFYKTLRAEQASQEVKNWMTETLLVQ |
| | | NANPDCKTILKALGPAATLEEMMTACQGVGGPGHKARVLAEAMSQVTNSATIML |
| | | QRMKQLEDKVEELLSKNYHLENEVARLKKLVGEASNFTQFVLVDNGGTGDVTVA |
| | | PSNFANGVAEWISSNSRSQAYKVTCSVRQSSAQKRKYTIKVEVPKVATQTVGGV |
| | | ELPVAAWRSYLNMELTIPIFATNSDCELIVKAMQGLLKDGNPIPSAIAANSGIY |
| | | FLGKIWPSYKGRPGNFLQSRPEPTAPPEESFRSGVETTTPPQKQEPIDKELYPL |
| | | TSLRSLFGNDPSSQ |
| GagZip-Δp1p6- | 22 | MGARASVLSGGELDRWEKIRLRPGGKKKYKLKHIVWASRELERFAVNPGLLETS |
| MCP | | EGCRQILGQLQPSLQTGSEELRSLYNTVATLYCVHQRIEIKDTKEALDKIEEEQ |
| | | NKSKKKAQQAAADTGHSNQVSQNYPIVQNIQGQMVHQAISPRTLNAWVKVVEEK |
| | | AFSPEVIPMFSALSEGATPQDLNTMLNTVGGHQAAMQMLKETINEEAAEWDRVH |
| | | PVHAGPIAPGQMREPRGSDIAGTTSTLQEQIGWMTNNPPIPVGEIYKRWIILGL |
| | | NKIVRMYSPTSILDIRQGPKEPFRDYVDRFYKTLRAEQASQEVKNWMTETLLVQ |
| | | NANPDCKTILKALGPAATLEEMMTACQGVGGPGHKARVLAEAMSQVTNSATIML |
| | | QRMKQLEDKVEELLSKNYHLENEVARLKKLVGEASNFTQFVLVDNGGTGDVTVA |
| | | PSNFANGVAEWISSNSRSQAYKVTCSVRQSSAQKRKYTIKVEVPKVATQTVGGV |
| | | ELPVAAWRSYLNMELTIPIFATNSDCELIVKAMQGLLKDGNPIPSAIAANSGIY |
| MiniGagZip- | 23 | MGARASVSPTSILDIRQGPKEPFRDYVDRFYKTLRAEQASQEVKNWMTETLLVQ |
| MCP | | NANPDCKTILKALGPAATLEEMMTACQGVGGPGHKARVLAEAMSQVTNSATIML |
| | | QRMKQLEDKVEELLSKNYHLENEVARLKKLVGELQSRPEPTAPPEESFRSGVET |
| | | TTPPQKQEPIDKELYPLTSLRSLFGNDPSSQASNFTQFVLVDNGGTGDVTVAPS |
| | | NFANGVAEWISSNSRSQAYKVTCSVRQSSAQKRKYTIKVEVPKVATQTVGGVEL |
| | | PVAAWRSYLNMELTIPIFATNSDCELIVKAMQGLLKDGNPIPSAIAANSGIY |
| MiniGagZip- | 24 | MGARASVSPTSILDIRQGPKEPFRDYVDRFYKTLRAEQASQEVKNWMTETLLVQ |
| MCPZip | | NANPDCKTILKALGPAATLEEMMTACQGVGGPGHKARVLAEAMSQVTNSATIML |
| | | QRMKQLEDKVEELLSKNYHLENEVARLKKLVGEASNFTQFVLVDNGGTGDVTVA |
| | | PSNFANGVAEWISSNSRSQAYKVTCSVRQSSAQKRKYTIKVEVPKVATQTVGGV |
| | | ELPVAAWRSYLNMELTIPIFATNSDCELIVKAMQGLLKDGNPIPSAIAANSGIY |

TABLE 2
RNA EXPORTERS

| Name | Component | Description |
|---|---|---|
| Gag | Exporter | Gag protein from HIV (Gag) |
| Gag-MCP | Exporter | Fusion of Gag and MS2 coat protein (MCP) with MCP |
| | | located after C-terminus of Gag |
| Gag-MCP_ZF2 | Exporter | Fusion of Gag and MCP with MCP located after second |
| | | zinc finger domain of Gag (ZF2) |
| Gag_dZF2-MCP | Exporter | Fusion of Gag and MCP with ZF2 deleted from Gag and |
| | | replaced by MCP |
| GagZip-MCP | Exporter | Fusion of Gag and MCP with nucleocapsid domain |
| | | deleted from Gag and replaced by leucine zipper from |
| | | GCN4 (GagZip), and MCP located after C-terminus of |
| | | Gag |
| GagZip-MCP_Zip | Exporter | Fusion of Gag and MCP with nucleocapsid domain |
| | | deleted from Gag and replaced by leucine zipper from |
| | | GCN4, and MCP located after leucine zipper |
| GagZip-dp1dp6-MCP | Exporter | Fusion of Gag and MCP with p1p6 domain deleted from |
| | | Gag, nucleocapsid domain deleted from Gag and replaced |
| | | by leucine zipper from GCN4, and MCP located after |
| | | leucine zipper |
| GagZip-MCP-dpol | Exporter | Fusion of Gag and MCP with nucleocapsid domain |
| | | deleted from Gag and replaced by leucine zipper from |
| | | GCN4 (GagZip), and MCP located after C-terminus of |
| | | Gag; coding sequence of Gag recoded to remove slippery |
| | | frameshift sequence at natural junction with Pol |
| MiniGagZip-MCP | Exporter | Fusion of Gag and MCP with portions of matrix, capsid, |
| | | and p1 domains deleted; nucleocapsid domain deleted and |
| | | replaced by leucine zipper from GCN4; and MCP located |
| | | after C terminus of Gag |
| MiniGagZip- | Exporter | Fusion of Gag and MCP with portions of matrix, capsid, |
| MCP_Zip | | and p1 domains deleted; nucleocapsid domain deleted and |
| | | replaced by leucine zipper from GCN4; and MCP located |
| | | after leucine zipper |

Disclosed herein include methods for determining sender cell-receiver cell dynamics. In some embodiments, the method comprises: contacting a population of sender cell(s) disclosed herein with a population of receiver cells, wherein the exported cargo RNA molecule(s) are thereby delivered to the receiver cells, and wherein the exported cargo RNA molecule(s) comprise one or more cell barcodes identifying the sender cell from which the exported cargo RNA molecule(s) are derived; and obtaining sequence information of the plurality of exported cargo RNA molecule(s), or products thereof, in each of the receiver cells to determine sender cell-receiver cell dynamics. The contacting can be in vivo (e.g., within a tissue). Obtaining sequence information can comprise sequencing or in situ hybridization In some embodiments, obtaining sequence information comprises, for each receiver cell, detecting the presence and/or amount of cell barcodes associated with each sender cell. In some embodiments, sender cell-receiver cell dynamics comprises: detecting of sender cell-receiver cell proximity; determining the relative positions of sender cells and receiver cells within a tissue and/or determining probing molecular transport rates within said tissue; and/or determining a three-dimensional physical map of the receiver cell population, sender cell population, and/or tissue.

Disclosed herein include systems for delivery of RNA molecules from sender cells to receiver cells. In some embodiments, the system comprises: an RNA exporter protein provided herein; cargo RNA molecule(s) provided herein; and a fusogen provided herein.

Disclosed herein include populations of lipid-enveloped nanoparticles (LNs). In some embodiments, the population of LNs comprises: an RNA exporter protein provided herein; cargo RNA molecule(s) provided herein; and a fusogen provided herein. The population of the LNs can be derived from expression of a nucleic acid composition provided herein.

In some embodiments, the sender cells and/or receiver cells comprise cells situated in an organ and/or tissue, e.g., an organ and/or tissue or a subject (e.g,. different organs and/or tissues of a subject). In some embodiments, the LNs contacted with RNase are capable of protecting cargo RNA molecule(s) comprised therein from RNase-mediated degradation (e.g., in the absence of detergent). In some embodiments, the average diameter of the LNs of the population of LNs is about 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 300 nm, 400 nm, or 500 nm. In some embodiments, the average is the mean (e.g., arithmetic mean, geometric mean, and/or harmonic mean), median or mode. In some embodiments, the LNs have a minimum diameter and/or a maximum diameter of about 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 300 nm, 400 nm, or 500 nm. In some embodiments, the diameter is hydrodynamic diameter, e.g., as measured by dynamic light scattering (DLS).

In some embodiments, the receiver cell or sender cell is: a cell of a subject, such as a subject suffering from a disease or disorder (e.g., a blood disease, an immune disease, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, or any combination thereof); a cell derived from a donor; and/or an in vivo cell, an ex vivo cell, or an in situ cell. In some embodiments, upon secretion from a sender cell, the LNs can be capable of distributing within one or more tissues of a subject. The receiver cells can be situated within one or more tissues of a subject. The one or more tissues can comprise adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue, fat tissue, or any combination thereof.

The sender cell and/or receiver cell can be a eukaryotic cell, such as a mammalian cell. The mammalian cell and/or the unique cell type can comprise an antigen-presenting cell, a dendritic cell, a macrophage, a neural cell, a brain cell, an astrocyte, a microglial cell, and a neuron, a spleen cell, a lymphoid cell, a lung cell, a lung epithelial cell, a skin cell, a keratinocyte, an endothelial cell, an alveolar cell, an alveolar macrophage, an alveolar pneumocyte, a vascular endothelial cell, a mesenchymal cell, an epithelial cell, a colonic epithelial cell, a hematopoietic cell, a bone marrow cell, a Claudius cell, Hensen cell, Merkel cell, Muller cell, Paneth cell, Purkinje cell, Schwann cell, Sertoli cell, acidophil cell, acinar cell, adipoblast, adipocyte, brown or white alpha cell, amacrine cell, beta cell, capsular cell, cementocyte, chief cell, chondroblast, chondrocyte, chromaffin cell, chromophobic cell, corticotroph, delta cell, Langerhans cell, follicular dendritic cell, enterochromaffin cell, ependymocyte, epithelial cell, basal cell, squamous cell, endothelial cell, transitional cell, erythroblast, erythrocyte, fibroblast, fibrocyte, follicular cell, germ cell, gamete, ovum, spermatozoon, oocyte, primary oocyte, secondary oocyte, spermatid, spermatocyte, primary spermatocyte, secondary spermatocyte, germinal epithelium, giant cell, glial cell, astroblast, astrocyte, oligodendroblast, oligodendrocyte, glioblast, goblet cell, gonadotroph, granulosa cell, haemocytoblast, hair cell, hepatoblast, hepatocyte, hyalocyte, interstitial cell, juxtaglomerular cell, keratinocyte, keratocyte, lemmal cell, leukocyte, granulocyte, basophil, eosinophil, neutrophil, lymphoblast, B-lymphoblast, T-lymphoblast, lymphocyte, B-lymphocyte, T-lymphocyte, helper induced T-lymphocyte, Th1 T-lymphocyte, Th2 T-lymphocyte, natural killer cell, thymocyte, macrophage, Kupffer cell, alveolar macrophage, foam cell, histiocyte, luteal cell, lymphocytic stem cell, lymphoid cell, lymphoid stem cell, macroglial cell, mammotroph, mast cell, medulloblast, megakaryoblast, megakaryocyte, melanoblast, melanocyte, mesangial cell, mesothelial cell, metamyelocyte, monoblast, monocyte, mucous neck cell, myoblast, myocyte, muscle cell, cardiac muscle cell, skeletal muscle cell, smooth muscle cell, myelocyte, myeloid cell, myeloid stem cell, myoblast, myoepithelial cell, myofibrobast, neuroblast, neuroepithelial cell, neuron, odontoblast, osteoblast, osteoclast, osteocyte, oxyntic cell, parafollicular cell, paraluteal cell, peptic cell, pericyte, peripheral blood mononuclear cell, phaeochromocyte, phalangeal cell, pinealocyte, pituicyte, plasma cell, platelet, podocyte, proerythroblast, promonocyte, promyeloblast, promyelocyte, pronormoblast, reticulocyte, retinal pigment epithelial cell, retinoblast, small cell, somatotroph, stem cell, sustentacular cell, teloglial cell, a zymogenic cell, or any combination thereof. The stem cell can comprise an embryonic stem cell, an induced pluripotent stem cell (iPSC), a hematopoietic stem/progenitor cell (HSPC), or any combination thereof.

The receiver cell can comprise a unique cell type and/or a unique cell state (e.g., a first cell type and/or a first cell state prior to fusing with the LNs). A unique cell type and/or a unique cell state can comprise a unique gene expression pattern. The unique cell type and/or unique cell state can comprise a unique anatomic location. In some embodiments, the unique cell type and/or the unique cell state can comprise anatomically locally unique gene expression. A unique cell type and/or a unique cell state can be caused by hereditable, environmental, and/or idiopathic factors. In some embodiments, the unique cell type and/or the cell in the unique cell state (i) causes and/or aggravates a disease or disorder and/or (ii) is associated with the pathology of a disease or disorder. The unique cell state can comprise a senescent cell state induced by a tumor microenvironment. The senescent cell state induced by a tumor microenvironment can comprise expression of CD57, KRLG1, TIGIT, or any combination thereof. The unique cell state and/or unique cell type can be characterized by aberrant signaling of one or more signal transducer(s). In some embodiments, the unique cell state comprises: a physiological state (e.g., a cell cycle state, a differentiation state, a development state a metabolic state, or a combination thereof); and/or a pathological state (e.g., a disease state, a human disease state, a diabetic state, an immune disorder state, a neurodegenerative disorder state, an oncogenic state, or a combination thereof).

The unique cell state and/or unique cell type can be characterized by one or more of cell proliferation, stress pathways, oxidative stress, stress kinase activation, DNA damage, lipid metabolism, carbohydrate regulation, metabolic activation including Phase I and Phase II reactions, Cytochrome P-450 induction or inhibition, ammonia detoxification, mitochondrial function, peroxisome proliferation, organelle function, cell cycle state, morphology, apoptosis, DNA damage, metabolism, signal transduction, cell differentiation, cell-cell interaction and cell to non-cellular compartment. The unique cell state and/or unique cell type can be characterized by one or more of acute phase stress, cell adhesion, AH-response, anti-apoptosis and apoptosis, antimetabolism, anti-proliferation, arachidonic acid release, ATP depletion, cell cycle disruption, cell matrix disruption, cell migration, cell proliferation, cell regeneration, cell-cell communication, cholestasis, differentiation, DNA damage, DNA replication, early response genes, endoplasmic reticulum stress, estogenicity, fatty liver, fibrosis, general cell stress, glucose deprivation, growth arrest, heat shock, hepatotoxicity, hypercholesterolemia, hypoxia, immunotox, inflammation, invasion, ion transport, liver regeneration, cell migration, mitochondrial function, mitogenesis, multidrug resistance, nephrotoxicity, oxidative stress, peroxisome damage, recombination, ribotoxic stress, sclerosis, steatosis, teratogenesis, transformation, disrupted translation, transport, and tumor suppression. The unique cell state and/or unique cell type can be characterized by one or more of nutrient deprivation, hypoxia, oxidative stress, hyperproliferative signals, oncogenic stress, DNA damage, ribonucleotide depletion, replicative stress, and telomere attrition, promotion of cell cycle arrest, promotion of DNA-repair, promotion of apoptosis, promotion of genomic stability, promotion of senescence, and promotion of autophagy, regulation of cell metabolic reprogramming, regulation of tumor microenvironment signaling, inhibition of cell stemness, survival, and invasion.

Synthetic biology allows for rational design of circuits that confer new functions in living cells. Many natural cellular functions are implemented by protein-level circuits, in which proteins specifically modify each other's activity, localization, or stability. Synthetic protein circuits have been described in, Gao, Xiaojing J., et al. “Programmable protein circuits in living cells.” Science 361.6408 (2018): 1252-1258; and WO2019/147478; the content of each of these, including any supporting or supplemental information or material, is incorporated herein by reference in its entirety. In some embodiments, synthetic protein circuits respond to inputs only above or below a certain tunable threshold concentration, such as those provided in US2020/0277333, the content of which is incorporated herein by reference in its entirety. In some embodiments, synthetic protein circuits comprise one or more synthetic protein circuit design components and/or concepts of US2020/0071362, the content of which is incorporated herein by reference in its entirety. In some embodiments, synthetic protein circuits comprise rationally designed circuits, including miRNA-level and/or protein-level incoherent feed-forward loop circuits, that maintain the expression of a payload at an efficacious level, such as those provided in US2021/0171582, the content of which is incorporated herein by reference in its entirety. The compositions, methods, systems and kits provided herein can be employed in concert with those described in International Patent Application No. PCT/US2021/048100, entitled “Synthetic Mammalian Signaling Circuits For Robust Cell Population Control” filed on Aug. 27, 2021, the content of which is incorporated herein by reference in its entirety. Said reference discloses circuits, compositions, nucleic acids, populations, systems, and methods enabling cells to sense, control, and/or respond to their own population size and can be employed with the circuits provided herein. In some embodiments, an orthogonal communication channel allows specific communication between engineered cells. Also described therein, in some embodiments, is an evolutionarily robust ‘paradoxical’ regulatory circuit architecture in which orthogonal signals both stimulate and inhibit net cell growth at different signal concentrations. In some embodiments, engineered cells autonomously reach designed densities and/or activate therapeutic or safety programs at specific density thresholds. The systems, methods, compositions, and kits provided herein can, in some embodiments, be employed in concert with the systems, methods, compositions, and kits described in PCT Patent Application Publication No. WO2022/125590, entitled, “A synthetic circuit for cellular multistability,” the content of which is incorporated herein by reference in its entirety. The systems, methods, compositions, and kits provided herein can, in some embodiments, be employed in concert with the systems, methods, compositions, and kits described in U.S. Patent Application No. 2018/0142307 and 2020/0172968, the contents of which are incorporated herein by reference in their entirety.

In some embodiments, the cargo RNA molecule(s) and/or payload protein(s) encoded by said cargo RNA molecule(s) constitute two or more components of a receiver circuit. The cargo RNA(s) and/or payload(s) can be capable of forming one or more receiver circuit(s) in a receiver cell. The receiver circuit can comprise cargo RNA molecules having molecular activity (e.g., a microRNA, an antagomir, an aptamer, and a ribozyme). The sender cell can comprise one or more fourth polynucleotide(s) encoding two or more components of a sender circuit. The receiver circuit and/or sender circuit can comprise one or more effector protein(s) and one or more modulator protein(s). The modulator protein(s) can be capable of regulating the expression, concentration, localization, stability, and/or activity the effector protein(s). In some embodiments, said regulating can be based on sensing of the cell type and/or cell state of a receiver cell and/or a sender cell.

A modulator protein can comprise a first protease, and an effector protein can comprise a cut site the first protease in the first protease active state is capable of cutting. The first protease can comprise tobacco etch virus (TEV) protease, tobacco vein mottling virus (TVMV) protease, hepatitis C virus protease (HCVP), derivatives thereof, or any combination thereof. In some embodiments, the effector protein changes from an effector inactive state to an effector active state when the first protease in the first protease active state cuts the first cut site of the effector. The effector protein can be changed into a first effector destabilized state, a first effector delocalized state, and/or a first effector inactivate state after the first protease in the first protease active state cuts the cut site of the effector protein. The effector protein can comprise a degron, the first protease in the first protease active state can be capable of cutting the second cut site of the effector protein to expose the degron, and the degron of the effector protein being exposed can change the effector protein to an effector destabilized state. The effector protein can comprise a degron, the first protease in the first protease active state can be capable of cutting the second cut site of the effector protein to hide the degron, and the degron of the effector protein being hidden can change the effector protein to an effector stabilized state. The degron can comprise an N-degron, a dihydrofolate reductase (DHFR) degron, a FKB protein (FKBP) degron, derivatives thereof, or any combination thereof.

The substrate of the effector protein(s) can comprise a nucleic acid, a protein, a lipid, or any combination thereof. The effector protein can be capable of changing a synthetic protein circuit component of the synthetic protein circuit to (i) a synthetic protein circuit component active state; or (ii) a synthetic protein circuit component inactive state. The effector protein in an effector active state can be capable of: activating an endogenous signal transduction pathway; inactivating an endogenous signal transduction pathway; and/or rendering a receiver cell sensitive to a prodrug. The expression, concentration, localization, stability, and/or activity the effector protein(s) can be related to a number of molecules of the signal transducer in a signal transducer active state. The expression and/or activity of the one or more payloads in the receiver cell can be conditional on the receiver cell type and/or receiver cell state.

The receiver circuit and/or sender circuit can be configured to be responsive to changes in: (i) cell environment, optionally cell environment comprises location relative to a target site of a subject and/or changes in the presence and/or absence of cell(s) of interest, optionally said cell(s) of interest comprise target-specific antigen(s); (ii) one or more signal transduction pathways regulating cell survival, cell growth, cell proliferation, cell adhesion, cell migration, cell metabolism, cell morphology, cell differentiation, apoptosis, or any combination thereof; (iii) input(s) of a synthetic receptor system, optionally Synthetic Notch (SynNotch) receptor, a Modular Extracellular Sensor Architecture (MESA) receptor, a synthekine, Tango, dCas9-synR, a chimeric antigen receptor, or any combination thereof and/or (iv) T cell activity, optionally T cell activity comprises one or more of T cell simulation, T cell activation, cytokine secretion, T cell survival, T cell proliferation, CTL activity, T cell degranulation, and T cell differentiation.

The receiver circuit(s) can be capable of modulating cell states, cell types, and/or cell behaviors. The receiver circuit(s) can be configured to selectively activate cell death and/or immune recruitment to tumor cells. The receiver circuit(s) can be configured to detect the intracellular state of the receiver cell and classifying it as tumor or normal based on the levels or activities of relevant molecules or pathways. The receiver circuit can be configured to reprogram a receiver cell type and/or cell state, such as, for example, via expression of transcription factors and/or epigenetic modifiers. The receiver circuit can be capable of directly or indirectly inducing cell death in the presence of the aberrant signaling of one or more signal transducer(s). The receiver circuit can be capable of detecting aberrant signaling, an activity of a signal transducer, an activity of a signal transducer activator and/or an activity of a signal transducer repressor. The detecting can comprise detecting a modification selected from the group comprising phosphorylation, dephosphorylation, acetylation, methylation, acylation, glycosylation, glycosylphosphatidylinositol (GPI) anchoring, sulfation, disulfide bond formation, deamidation, ubiquitination, sumoylation, nitration of tyrosine, hydrolysis of ATP or GTP, binding of ATP or GTP, cleavage, or any combination thereof. The receiver circuit(s) can be capable of reprogramming a receiver cell from a first cell type and/or first cell state to a second cell type and/or second cell state. The effector protein in the effector active state, or the effector inactive state, can be capable of changing a state of the cell, thereby treating a disease or disorder characterized by the aberrant signaling of a signal transducer.

In some embodiments, the sender cell is not capable of releasing the LNs when the sender cell does not induce expression of the RNA exporter protein. A sender circuit can be capable of modulating the expression, concentration, localization, stability, and/or activity of the RNA exporter protein and/or the fusogen. In some embodiments, first promoter(s) can be operably linked to each of the one or more first polynucleotide(s), and wherein a first promoter is capable of inducing transcription of a first polynucleotide to generate an RNA exporter transcript. In some embodiments, one or more first promoter(s) can comprise a heterologous promoter element and/or an endogenous promoter element. A heterologous promoter element can be capable of being bound by an effector protein of the sender circuit (e.g., a transcription factor); and/or an endogenous promoter element can be capable of being bound by an endogenous protein of a cell. The RNA exporter protein can be constitutively expressed by the sender cell(s). The sender cell can be configured such that the activation and/or degree of: fusogen expression; RNA exporter protein expression: and/or LN secretion from a sender cell is dependent on: (i) endogenous signals, optionally the local environment of the sender cell, further optionally cell-surface or soluble molecules, extracellular structures, physical or chemical properties, or combinations thereof; and/or (ii) exogenous signals, optionally light, heat, ultrasound, small molecule (e.g., transactivator), or a combination thereof. In some embodiments, one or more first polynucleotide(s) can comprise one or more silencer effector binding sequences. The silencer effector can comprise a microRNA (miRNA), a precursor microRNA (pre-miRNA), a small interfering RNA (siRNA), a short-hairpin RNA (shRNA), precursors thereof, derivatives thereof, or a combination thereof. The silencer effector can be capable of binding the one or more silencer effector binding sequences, thereby reducing the stability of RNA exporter transcript(s) The expression and/or activity of the silencer effector can be configured to be responsive to changes in endogenous signals, and/or exogenous signals. In some embodiments, one or more first promoter(s) can comprise one or more copies of a transactivator recognition sequence that a transactivator is capable of binding and wherein, in the presence of the transactivator and a transactivator-binding compound, the first promoter is capable of inducing transcription of the first polynucleotides(s). The transactivator recognition sequence can comprise a Tet3G binding site (TRE3G) or a ERT2-Gal4 binding site (UAS). The transactivator-binding compound can comprise 4-hydroxy-tamoxifen (4-OHT), Dox, derivatives thereof, or any combination thereof. A transactivator recognition sequence can comprise an element of an inducible promoter. The inducible promoter can be a tetracycline responsive promoter, a TRE promoter, a Tre3G promoter, an ecdysone responsive promoter, a cumate responsive promoter, a glucocorticoid responsive promoter, and estrogen responsive promoter, a PPAR-γ promoter, or an RU-486 responsive promoter.

The fusogen can be configured to bind one or more receiver cells of a subject. A payload protein can comprise one or more receptors and/or a targeting moiety configured to bind a component of a target site of a subject. The sender sends can be configured to travel to and/or accumulate at a target site of a subject. The LNs can comprise one or more targeting moieties configured to bind: (i) a target site of a subject; and/or (ii) a first antigen of target receiver cells. A target site of a subject: can comprise target receiver cells; and/or can be a site of disease or disorder or can be proximate to a site of a disease or disorder. The one or more targeting moieties can be selected from the group comprising mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, and an RGD peptide or RGD peptide mimetic. The one or more targeting moieties can comprise one or more of the following: an antibody or antigen-binding fragment thereof, a peptide, a polypeptide, an enzyme, a peptidomimetic, a glycoprotein, a lectin, a nucleic acid, a monosaccharide, a disaccharide, a trisaccharide, an oligosaccharide, a polysaccharide, a glycosaminoglycan, a lipopolysaccharide, a lipid, a vitamin, a steroid, a hormone, a cofactor, a receptor, a receptor ligand, and analogs and derivatives thereof. The one or more targeting moieties can be configured to bind/associate with the RNA exporter protein, or can be separate from the RNA exporter protein. The one or more targeting moieties can be present in the LNs provided herein, thereby directing the LNs to target cell(s) (e.g., receiver cells of interest).

The one or more targeting moieties can be configured to bind one or more of the following: CD3, CD4, CD5, CD6, CD7, CD8, CD9, CD10, CD11a, CD11b, CD11c, CD12w, CD14, CD15, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD46, CD47, CD48, CD49b, CD49c, CD51, CD52, CD53, CD54, CD55, CD56, CD58, CD59, CD61, CD62E, CD62L, CD62P, CD63, CD66, CD68, CD69, CD70, CD72, CD74, CD79, CD79a, CD79b, CD80, CD81, CD82, CD83, CD86, CD87, CD88, CD89, CD90, CD91, CD95, CD96, CD98, CD100, CD103, CD105, CD106, CD109, CD117, CD120, CD125, CD126, CD127, CD133, CD134, CD135, CD137, CD138, CD141, CD142, CD143, CD144, CD147, CD151, CD147, CD152, CD154, CD156, CD158, CD163, CD166, CD168, CD174, CD180, CD184, CDw186, CD194, CD195, CD200, CD200a, CD200b, CD209, CD221, CD227, CD235a, CD240, CD262, CD271, CD274, CD276 (B7-H3), CD303, CD304, CD309, CD326, 4-1BB, 5 AC, 5T4 (Trophoblast glycoprotein, TPBG, 5T4, Wnt-Activated Inhibitory Factor 1 or WAIF1), Adenocarcinoma antigen, AGS-5, AGS-22M6, Activin receptor like kinase 1, AFP, AKAP-4, ALK, Alpha integrin, Alpha v beta6, Amino-peptidase N, Amyloid beta, Androgen receptor, Angiopoietin 2, Angiopoietin 3, Annexin A1, Anthrax toxin protective antigen, Anti-transferrin receptor, AOC3 (VAP-1), B7-H3, Bacillus anthracis anthrax, BAFF (B-cell activating factor), B-lymphoma cell, bcr-abl, Bombesin, BORIS, C5, C242 antigen, CA125 (carbohydrate antigen 125, MUC16), CA-IX (CAIX, carbonic anhydrase 9), CALLA, CanAg, Canis lupus familiaris IL31, Carbonic anhydrase IX, Cardiac myosin, CCL11 (C—C motif chemokine 11), CCR4 (C—C chemokine receptor type 4, CD194), CCR5, CD3E (epsilon), CEA (Carcinoembryonic antigen), CEACAM3, CEACAM5 (carcinoembryonic antigen), CFD (Factor D), Ch4D5, Cholecystokinin 2 (CCK2R), CLDN18 (Claudin-18), Clumping factor A, CRIPTO, FCSF1R (Colony stimulating factor 1 receptor, CD 115), CSF2 (colony stimulating factor 2, Granulocyte-macrophage colony-stimulating factor (GM-CSF)), CTLA4 (cytotoxic T-lymphocyte-associated protein 4), CTAA16.88 tumor antigen, CXCR4 (CD 184), C—X—C chemokine receptor type 4, cyclic ADP ribose hydrolase, Cyclin B1, CYP1B1, Cytomegalovirus, Cytomegalovirus glycoprotein B, Dabigatran, DLL4 (delta-like—ligand 4), DPP4 (Dipeptidyl-peptidase 4), DR5 (Death receptor 5), E. coli Shiga toxin type-1, E. coli Shiga toxin type-2, ED-B, EGFL7 (EGF-like domain-containing protein 7), EGFR, EGFRII, EGFRvIII, Endoglin (CD 105), Endothelin B receptor, Endotoxin, EpCAM (epithelial cell adhesion molecule), EphA2, Episialin, ERBB2 (Epidermal Growth Factor Receptor 2), ERBB3, ERG (TMPRSS2 ETS fusion gene), Escherichia coli, ETV6-AML, FAP (Fibroblast activation protein alpha), FCGR1, alpha-Fetoprotein, Fibrin II, beta chain, Fibronectin extra domain-B, FOLR (folate receptor), Folate receptor alpha, Folate hydrolase, Fos-related antigen 1.F protein of respiratory syncytial virus, Frizzled receptor, Fucosyl GM1, GD2 ganglioside, G-28 (a cell surface antigen glycolipid), GD3 idiotype, GloboH, Glypican 3, N-glycolylneuraminic acid, GM3, GMCSF receptor a-chain, Growth differentiation factor 8, GP100, GPNMB (Transmembrane glycoprotein NMB), GUCY2C (Guanylate cyclase 2C, guanylyl cyclase C (GC-C), intestinal Guanylate cyclase, Guanylate cyclase-C receptor, Heat-stable enterotoxin receptor (hSTAR)), Heat shock proteins, Hemagglutinin, Hepatitis B surface antigen, Hepatitis B virus, HER1 (human epidermal growth factor receptor 1), HER2, HER2/neu, HER3 (ERBB-3), IgG4, HGF/SF (Hepatocyte growth factor/scatter factor), HHGFR, HIV-1, Histone complex, HLA-DR (human leukocyte antigen), HLA-DR10, HLA-DRB, HMWMAA, Human chorionic gonadotropin, HNGF, Human scatter factor receptor kinase, HPV E6/E7, Hsp90, hTERT, ICAM-1 (Intercellular Adhesion Molecule 1), Idiotype, IGF1R (IGF-1, insulin-like growth factor 1 receptor), IGHE, IFN-γ, Influenza hemagglutinin, IgE, IgE Fc region, IGHE, IL-1, IL-2 receptor (interleukin 2 receptor), IL-4, IL-5, IL-6, IL-6R (interleukin 6 receptor), IL-9, IL-10, IL-12, IL-13, IL-17, IL-17A, IL-20, IL-22, IL-23, IL31RA, ILGF2 (Insulin-like growth factor 2), Integrins (α4, αₙβ₃, ανβ3, α₄β₇, α5β1, α6β4, α7β7, α11β3, α5β5, ανβ5), Interferon gamma-induced protein, ITGA2, ITGB2, KIR2D, LCK, Le, Legumain, Lewis-Y antigen, LFA-l (Lymphocyte function-associated antigen 1, CD11a), LHRH, LINGO-1, Lipoteichoic acid, LIV1A, LMP2, LTA, MAD-CT-1, MAD-CT-2, MAGE-1, MAGE-2, MAGE-3, MAGE Al, MAGE A3, MAGE 4, MARTI, MCP-1, MIF (Macrophage migration inhibitory factor, or glycosylation inhibiting factor (GIF)), MS4A1 (membrane-spanning 4-domains subfamily A member 1), MSLN (mesothelin), MUCl (Mucin 1, cell surface associated (MUC1) or polymorphic epithelial mucin (PEM)), MUC1-KLH, MUC16 (CA125), MCPl (monocyte chemotactic protein 1), MelanA/MART1, ML-IAP, MPG, MS4A1 (membrane-spanning 4-domains subfamily A), MYCN, Myelin-associated glycoprotein, Myostatin, NA17, NARP-1, NCA-90 (granulocyte antigen), Nectin-4 (ASG-22ME), NGF, Neural apoptosis-regulated proteinase 1, NOGO-A, Notch receptor, Nucleolin, Neu oncogene product, NY-BR-1, NY-ESO-1, OX-40, OxLDL (Oxidized low-density lipoprotein), OY-TES 1, P21, p53 nonmutant, P97, Page 4, PAP, Paratope of anti-(N-glycolylneuraminic acid), PAX3, PAX5, PCSK9, PDCD1 (PD-1, Programmed cell death protein 1, CD279), PDGF-Ra (Alpha-type platelet-derived growth factor receptor), PDGFR-β, PDL-1, PLAC1, PLAP-like testicular alkaline phosphatase, Platelet-derived growth factor receptor beta, Phosphate-sodium co-transporter, PMEL 17, Polysialic acid, Proteinase3 (PR1), Prostatic carcinoma, PS (Phosphatidylserine), Prostatic carcinoma cells, Pseudomonas aeruginosa, PSMA, PSA, PSCA, Rabies virus glycoprotein, RHD (Rh polypeptide 1 (RhPI), CD240), Rhesus factor, RANKL, RhoC, Ras mutant, RGS5, ROBO4, Respiratory syncytial virus, RON, Sarcoma translocation breakpoints, SART3, Sclerostin, SLAMF7 (SLAM family member 7), Selectin P, SDC1 (Syndecan 1), sLe(a), Somatomedin C, SIP (Sphingosine-1-phosphate), Somatostatin, Sperm protein 17, SSX2, STEAP1 (six-transmembrane epithelial antigen of the prostate 1), STEAP2, STn, TAG-72 (tumor associated glycoprotein 72), Survivin, T-cell receptor, T cell transmembrane protein, TEM1 (Tumor endothelial marker 1), TENB2, Tenascin C (TN-C), TGF-a, TGF-β (Transforming growth factor beta), TGF-βI, TGF-β2 (Transforming growth factor-beta 2), Tie (CD202b), Tie2, TIM-1 (CDX-014), Tn, TNF, TNF-a, TNFRSF8, TNFRSF10B (tumor necrosis factor receptor superfamily member 10B), TNFRSF13B (tumor necrosis factor receptor superfamily member 13B), TPBG (trophoblast glycoprotein), TRAIL-R1 (Tumor necrosis apoptosis Inducing ligand Receptor 1), TRAILR2 (Death receptor 5 (DR5)), tumor-associated calcium signal transducer 2, tumor specific glycosylation of MUC1, TWEAK receptor, TYRP1 (glycoprotein 75), TRP-2, Tyrosinase, VCAM-1 (CD 106), VEGF, VEGF-A, VEGF-2 (CD309), VEGFR-1, VEGFR2, or vimentin, WT1, XAGE 1, or cells expressing any insulin growth factor receptors, or any epidermal growth factor receptors.

The composition can comprise: a bridge protein. The sender cell can be capable of expressing a bridge protein. A bridge protein can comprise a fusogen-binding domain and a first antigen-binding domain (e.g., the one or more targeting moieties disclosed herein). A first antigen-binding moiety of the bridge protein can be capable of binding a first antigen on a surface of a target receiver cell. A fusogen-binding domain of the bridge protein can be capable of binding a fusogen on a surface of a LN. The LN can be capable of fusing with a receiver cell when the first antigen-binding moiety is bound to the first antigen and the fusogen-binding domain is bound to the fusogen.

In some embodiments, the one or more of one or more of the first polynucleotide(s), the second polynucleotide(s), the third polynucleotide(s), and the fourth polynucleotide(s), is between about 30 and 100000 nucleotides in length. The payload protein(s) can be between about 30 amino acids and 3000 amino acids in length. In some embodiments, one or more of one or more of the first polynucleotide(s), the second polynucleotide(s), the third polynucleotide(s), and the fourth polynucleotide(s), comprise: a 5′UTR and/or a 3′UTR; a tandem gene expression element selected from the group an internal ribosomal entry site (IRES), foot-and-mouth disease virus 2A peptide (F2A), equine rhinitis A virus 2A peptide (E2A), porcine teschovirus 2A peptide (P2A) or Thosea asigna virus 2A peptide (T2A), or any combination thereof; and/or a transcript stabilization element (e.g., woodchuck hepatitis post-translational regulatory element (WPRE), bovine growth hormone polyadenylation (bGH-polyA) signal sequence, human growth hormone polyadenylation (hGH-polyA) signal sequence, or any combination thereof).

The nucleic acid composition and/or cargo RNA molecule(s) can be configured to enhance stability, durability, and/or expression level, optionally a 5′ untranslated region (UTR), a 3′ UTR, and/or a 5′ cap; optionally one or more modified nucleotides, further optionally selected from the group comprising pseudouridine, N-1-methyl-pseudouridine, 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine; and/or optionally a modified nucleotide in place of one or more uridines, optionally the modified nucleoside is selected from pseudouridine (ψ), N1-methyl-pseudouridine (m PP), and 5-methyl-uridine (m5U).

In some embodiments, one or more of one or more of the first polynucleotide(s), the second polynucleotide(s), the third polynucleotide(s), and the fourth polynucleotide(s), can be operably connected to a promoter selected from the group comprising: a minimal promoter, optionally TATA, miniCMV, and/or miniPromo; a ubiquitous promoter; a tissue-specific promoter and/or a lineage-specific promoter; and/or a ubiquitous promoter (e.g., a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CASI promoter, a CBH promoter, or any combination thereof).

The nucleic acid composition can be complexed or associated with one or more lipids or lipid-based carriers, thereby forming liposomes, lipid nanoparticles (LNPs), lipoplexes, and/or nanoliposomes, optionally encapsulating the nucleic acid composition. In some embodiments, the nucleic acid composition is, comprises, or further comprises, one or more vectors. At least one of the one or more vectors can be a viral vector, a plasmid, a transposable element, a naked DNA vector, a lipid nanoparticle (LNP), or any combination thereof. The viral vector can be an AAV vector, a lentivirus vector, a retrovirus vector, an adenovirus vector, a herpesvirus vector, a herpes simplex virus vector, a cytomegalovirus vector, a vaccinia virus vector, a MVA vector, a baculovirus vector, a vesicular stomatitis virus vector, a human papillomavirus vector, an avipox virus vector, a Sindbis virus vector, a VEE vector, a Measles virus vector, an influenza virus vector, a hepatitis B virus vector, an integration-deficient lentivirus (IDLV) vector, or any combination thereof. The transposable element can be piggybac transposon or sleeping beauty transposon. The polynucleotide(s) encoding the RNA exporter protein, the fusogen, and/or the cargo RNA molecule(s) can be comprised in the one or more vectors. The polynucleotide(s) encoding the RNA exporter protein, the fusogen, and/or the cargo RNA molecule(s) can be comprised in the same vector and/or different vectors. The polynucleotide(s) encoding the RNA exporter protein, the fusogen, and/or the cargo RNA molecule(s) can be situated on the same nucleic acid and/or different nucleic acids. The payload protein(s), fusogen, and/or RNA exporter protein can be configured to exhibit minimal immunogenicity in a subject, and in some embodiments can be derived from commensal viruses or endogenous viruses and/or can be novo designed proteins and/or and humanized.

Vectors provided herein include integrating vectors and non-integrating vectors. Integrating vectors have their delivered RNA/DNA permanently incorporated into the host cell chromosomes. Non-integrating vectors remain episomal which means the nucleic acid contained therein is never integrated into the host cell chromosomes. Examples of integrating vectors include retroviral vectors, lentiviral vectors, hybrid adenoviral vectors, and herpes simplex viral vector. One example of a non-integrative vector is a non-integrative viral vector. Non-integrative viral vectors eliminate the risks posed by integrative retroviruses, as they do not incorporate their genome into the host DNA. One example is the Epstein Barr oriP/Nuclear Antigen-1 (“EBNAl”) vector, which is capable of limited self-replication and known to function in mammalian cells. As containing two elements from Epstein-Barr virus, oriP and EBNAl, binding of the EBNAl protein to the virus replicon region oriP maintains a relatively long-term episomal presence of plasmids in mammalian cells. This particular feature of the oriP/EBNAl vector makes it ideal for generation of integration-free iPSCs. Another non-integrative viral vector is adenoviral vector and the adeno-associated viral (AAV) vector. Other non-integrative viral vectors contemplated herein are single-strand negative-sense RNA viral vectors, such Sendai viral vector and rabies viral vector. Another example of a non-integrative vector is a minicircle vector. Minicircle vectors are circularized vectors in which the plasmid backbone has been released leaving only the eukaryotic promoter and cDNA(s) that are to be expressed. As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of nonessential viral genes. The vector and/or particle may be utilized for the purpose of transferring nucleic acids into cells either in vitro or in vivo. Numerous forms of viral vectors are known in the art.

### Payload Proteins

The cargo RNA molecule(s) can be mRNA. The packing signal(s) can be situated in the 5′UTR and/or 3′UTR. The cargo RNA molecule(s) can be transcribed from a RNA polymerase I promoter, a RNA polymerase II promoter, a RNA polymerase III promoter, a T7 promoter, a T3 promoter, or any combination thereof. In some embodiments, one or more cargo RNA molecule(s) encode one or more payload protein(s), and wherein said payload proteins can be capable of being translated upon delivery to the receiver cell(s). A payload protein can be capable of modulating the expression, concentration, localization, stability, and/or activity of the one or more endogenous proteins of a receiver cell. The payload protein can be a therapeutic protein or a variant thereof. The therapeutic protein can be configured to prevent or treat a disease or disorder of a subject, and, in some embodiments, the subject suffers from a deficiency of said therapeutic protein. Payload proteins can be effector proteins of a circuit.

In some embodiments, the cargo RNA molecule(s) are capable of inducing the receiver cell to express bystander-modulating molecules, wherein bystander-modulating molecules are molecules modulating bystander cells that have not been delivered the cargo RNA molecule(s), and wherein the cargo RNA molecule(s) encode bystander-modulating molecules; or wherein the cargo RNA molecule(s), payload protein(s), and/or receiver circuit(s) are capable of inducing the receiver cell to express endogenous bystander-modulating molecules. The bystander-modulating molecules can comprise cytokines, morphogens, ligands, cell-surface molecules, or any combination thereof. The cargo RNA molecule(s) can be covalently and/or noncovalently attached to companion molecule(s). The companion molecule(s) can be a nucleic acid, an antibody or a portion thereof, an antibody-like molecule, an enzyme, a cell, an antigen, a small molecule, a protein, a peptide, a peptidomimetic, a sugar, a carbohydrate, a lipid, a glycan, a glycoprotein, an aptamer, or any combination thereof.

A payload protein can comprise an agonistic or antagonistic antibody or antigen-binding fragment thereof specific to a checkpoint inhibitor or checkpoint stimulator molecule (e.g., PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, and/or TIM-3). The one or more payloads can comprise a secretion tag. The secretion tag can be selected from the group comprising AbnA, AmyE, AprE, BglC, BglS, Bpr, Csn, Epr, Ggt, GlpQ, HtrA, LipA, LytD, MntA, Mpr, NprE, OppA, PbpA, PbpX, Pel, PelB, PenP, PhoA, PhoB, PhoD, PstS, TasA, Vpr, WapA, WprA, XynA, XynD, YbdN, Ybxl, YcdH, YclQ, YdhF, YdhT, YfkN, YflE, YfmC, Yfnl, YhcR, YlqB, YncM, YnfF, YoaW, YocH, YolA, YqiX, Yqxl, YrpD, YrpE, YuaB, Yurl, YvcE, YvgO, YvpA, YwaD, YweA, YwoF, YwtD, YwtF, YxaLk, YxiA, and YxkC. A payload protein can comprise a constitutive signal peptide for protein degradation (e.g., PEST). A payload protein can comprise a nuclear localization signal (NLS) or a nuclear export signal (NES). A payload protein can comprise a dosage indicator protein. The dosage indicator protein can be detectable. The dosage indicator protein can comprise green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof.

The payload protein(s) can be configured to reduce their expression, localization, stability, and/or activity in sender cells. The sender cells express a second protease, payload protein(s) can comprise a cut site the second protease in the second protease active state is capable of cutting to reduce the stability, localization, and/or activity of the payload protein(s). In some embodiments, receiver cells does not comprise the second protease. The second protease can comprise tobacco etch virus (TEV) protease, tobacco vein mottling virus (TVMV) protease, hepatitis C virus protease (HCVP), derivatives thereof, or any combination thereof. In some embodiments, the second protease cutting the cut site exposes a degron. The degron can comprise an N-degron, a dihydrofolate reductase (DHFR) degron, a FKB protein (FKBP) degron, derivatives thereof, or any combination thereof. In some embodiments, one or more of the payload protein(s) is a degron fusion protein comprising a degron capable of binding a degron stabilizing molecule, and wherein the degron fusion protein changes from a destabilized state to a stabilized state when the degron binds to the degron stabilizing molecule, and wherein the degron stabilizing molecule is: an endogenous molecule of a receiver cell; a molecule absent in a sender cell; is a molecule specific to a cell type; is a molecule specific to a disease or disorder; and/or is a synthetic protein circuit component. In some embodiments, one or more of the payload protein(s) is a conditionally stable fusion protein comprising a stabilizing molecule binding domain capable of binding a stabilizing molecule, and wherein the conditionally stable fusion protein changes from a destabilized state to a stabilized state when the stabilizing molecule binding domain binds to the stabilizing molecule, and wherein the stabilizing molecule is: an endogenous molecule of a receiver cell; a molecule absent in a sender cell; is a molecule specific to a cell type; is a molecule specific to a disease or disorder; and/or is a synthetic protein circuit component. The stabilizing molecule binding domain can comprise a Camel Ig, Ig NAR, Fab fragments, Fab′ fragments, F(ab)′2 fragments, F(ab)′3 fragments, Fv, single chain Fv antibody (“scFv”), bis-scFv, (scFv)2, minibody, diabody, triabody, tetrabody, disulfide stabilized Fv protein (“dsFv”), single-domain antibody (sdAb), or any combination thereof.

The payload protein can comprise a synthetic protein circuit component. In some embodiments, the payload comprises a bispecific T cell engager (BiTE). In some embodiments, the orthogonal signal triggers cellular differentiation. The payload protein can comprise fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. The payload protein can comprise nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. The payload protein can comprise a CRE recombinase, GCaMP, a cell therapy component, a knock-down gene therapy component, a cell-surface exposed epitope, or any combination thereof. The payload protein can comprise a diagnostic agent (e.g., green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof).

In some embodiments, the payload protein can diminish immune cell function. The payload protein can be an activity regulator. The activity regulator can be capable of reducing T cell activity. The activity regulator can comprise a ubiquitin ligase involved in TCR/CAR signal transduction selected from the group comprising c-CBL, CBL-B, ITCH, R F125, R F128, WWP2, or any combination thereof. The activity regulator can comprise a negative regulatory enzyme selected from the group comprising SHP1, SHP2, SHTP1, SHTP2, CD45, CSK, CD148, PTPN22, DGKalpha, DGKzeta, DRAK2, HPK1, HPK1, STS1, STS2, SLAT, or any combination thereof. The activity regulator can be a negative regulatory scaffold/adapter protein selected from the group comprising PAG, LIME, NTAL, LAX31, SIT, GAB2, GRAP, ALX, SLAP, SLAP2, DOK1, DOK2, or any combination thereof. The activity regulator can be a dominant negative version of an activating TCR signaling component selected from the group comprising ZAP70, LCK, FYN, NCK, VAV1, SLP76, ITK, ADAP, GADS, PLCgammal, LAT, p85, SOS, GRB2, NFAT, p50, p65, API, RAPl, CRKII, C3G, WAVE2, ARP2/3, ABL, ADAP, RIAM, SKAP55, or any combination thereof. The activity regulator can comprise the cytoplasmic tail of a negative co-regulatory receptor selected from the group comprising CD5, PD1, CTLA4, BTLA, LAG3, B7-H1, B7-1, CD160, TFM3, 2B4, TIGIT, or any combination thereof. The activity regulator can be targeted to the plasma membrane with a targeting sequence derived from LAT, PAG, LCK, FYN, LAX, CD2, CD3, CD4, CD5, CD7, CD8a, PD1, SRC, LYN, or any combination thereof. In some embodiments, the activity regulator reduces or abrogates a pathway and/or a function selected from the group comprising Ras signaling, PKC signaling, calcium-dependent signaling, NF-kappaB signaling, NFAT signaling, cytokine secretion, T cell survival, T cell proliferation, CTL activity, degranulation, tumor cell killing, differentiation, or any combination thereof.

The payload protein can comprise a cytokine. The cytokine can be selected from the group consisting of interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF, or receptor or ligand thereof.

The payload protein can comprise a member of the TGF-β/BMP family selected from the group consisting of TGF-β1, TGF-β2, TGF-β3, BMP-2, BMP-3a, BMP-3b, BMP-4, BMP-5, BMP-6, BMP-7, BMP-8a, BMP-8b, BMP-9, BMP-10, BMP-11, BMP-15, BMP-16, endometrial bleeding associated factor (EBAF), growth differentiation factor-1 (GDF-1), GDF-2, GDF-3, GDF-5, GDF-6, GDF-7, GDF-8, GDF-9, GDF-12, GDF-14, mullerian inhibiting substance (MIS), activin-1, activin-2, activin-3, activin-4, and activin-5. The payload protein can comprise a member of the TNF family of cytokines selected from the group consisting of TNF-alpha, TNF-beta, LT-beta, CD40 ligand, Fas ligand, CD 27 ligand, CD 30 ligand, and 4-1 BBL. The payload protein can comprise a member of the immunoglobulin superfamily of cytokines selected from the group consisting of B7.1 (CD80) and B7.2 (B70). The payload protein can comprise an interferon. The interferon can be selected from interferon alpha, interferon beta, or interferon gamma. The payload protein can comprise a chemokine. The chemokine can be selected from CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13, or CXCL15. The payload protein can comprise a interleukin. The interleukin can be selected from IL-10 IL-12, IL-1, IL-6, IL-7, IL-15, IL-2, IL-18 or IL-21. The payload protein can comprise a tumor necrosis factor (TNF). The TNF can be selected from TNF-alpha, TNF-beta, TNF-gamma, CD252, CD154, CD178, CD70, CD153, or 4-1BBL.

The payload protein can comprise a programmable nuclease. In some embodiments, the receiver cell circuit senses correction of an aberrant locus by said programmable nuclease and reduces effector protein localization and/or activity. In some embodiments, the programmable nuclease is selected from the group comprising: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions. The programmable nuclease can comprise a zinc finger nuclease (ZFN) and/or transcription activator-like effector nuclease (TALEN). The programmable nuclease can comprise Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cash, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof. In some embodiments, the cargo RNA molecule(s) further comprise a polynucleotide encoding (i) a targeting molecule and/or (ii) a donor nucleic acid. In some embodiments, the targeting molecule is capable of associating with the programmable nuclease. In some embodiments, wherein the targeting molecule comprises single strand DNA or single strand RNA. In some embodiments, the targeting molecule comprises a single guide RNA (sgRNA).

In some embodiments, the payload protein is a therapeutic protein or variant thereof. Non-limiting examples of therapeutic proteins include blood factors, such as β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-receptors, soluble VEGF receptors, soluble interleukin receptors (e.g., soluble IL-1 receptors and soluble type II IL-1 receptors), soluble γ/δ T cell receptors, ligand-binding fragments of a soluble receptor, and the like; enzymes, such as—glucosidase, imiglucarase, β-glucocerebrosidase, and alglucerase; enzyme activators, such as tissue plasminogen activator; chemokines, such as IP-10, monokine induced by interferon-gamma (Mig), Gro/IL-8, RANTES, MIP-l, MIP-I β, MCP-1, PF-4, and the like; angiogenic agents, such as vascular endothelial growth factors (VEGFs, e.g., VEGF121, VEGF165, VEGF-C, VEGF-2), transforming growth factor-beta, basic fibroblast growth factor, glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); transforming growth factors (TGFs); tissue factors, luteinizing hormone; macrophage activating factors; tumor necrosis factor (TNF); neutrophil chemotactic factor (NCF); nerve growth factor; tissue inhibitors of metalloproteinases; vasoactive intestinal peptide; angiogenin; angiotropin; fibrin; hirudin; IL-1 receptor antagonists; and the like. Some other non-limiting examples of payload protein include ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); hemophilia related clotting proteins, such as Factor VIII, Factor IX, Factor X; dystrophin or mini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GLUT2), aldolase A, β-enolase, and glycogen synthase; lysosomal enzymes (e.g., beta-N-acetylhexosaminidase A); and any variants thereof.

In some embodiments, the payload protein is an active fragment of a protein, such as any of the aforementioned proteins. In some embodiments, the payload protein is a fusion protein comprising some or all of two or more proteins. In some embodiments a fusion protein can comprise all or a portion of any of the aforementioned proteins.

In some embodiments, the payload protein is a multi-subunit protein. For examples, the payload protein can comprise two or more subunits, or two or more independent polypeptide chains. In some embodiments, the payload protein can be an antibody. Examples of antibodies include, but are not limited to, antibodies of various isotypes (for example, IgG1, IgG2, IgG3, IgG4, IgA, IgD, IgE, and IgM); monoclonal antibodies produced by any means known to those skilled in the art, including an antigen-binding fragment of a monoclonal antibody; humanized antibodies; chimeric antibodies; single-chain antibodies; antibody fragments such as Fv, F(ab′)2, Fab′, Fab, Facb, scFv and the like; provided that the antibody is capable of binding to antigen. In some embodiments, the antibody is a full-length antibody.

In some embodiments, the payload protein is a pro-survival protein (e.g., Bcl-2, Bcl-XL, Mcl-1 and A1). In some embodiments, the payload protein comprises a apoptotic factor or apoptosis-related protein such as, for example, AIF, Apaf (e.g., Apaf-1, Apaf-2, and Apaf-3), oder APO-2 (L), APO-3 (L), Apopain, Bad, Bak, Bax, Bcl-2, Bcl-x_{L}, Bcl-x_{S}, bik, CAD, Calpain, Caspase (e.g., Caspase-1, Caspase-2, Caspase-3, Caspase-4, Caspase-5, Caspase-6, Caspase-7, Caspase-8, Caspase-9, Caspase-10, and Caspase-11), ced-3, ced-9, c-Jun, c-Myc, crm A, cytochrom C, CdR1, DcR1, DD, DED, DISC, DNA-PKcs, DR3, DR4, DR5, FADD/MORT-1, FAK, Fas (Fas-ligand CD95/fas (receptor)), FLICE/MACH, FLIP, fodrin, fos, G-Actin, Gas-2, gelsolin, granzyme A/B, ICAD, ICE, JNK, Lamin A/B, MAP, MCL-1, Mdm-2, MEKK-1, MORT-1, NEDD, NF-ₖₐₚₚₐB, NuMa, p53, PAK-2, PARP, perforin, PITSLRE, PKCdelta, pRb, presenilin, prICE, RAIDD, Ras, RIP, sphingomyelinase, thymidinkinase from herpes simplex, TRADD, TRAF2, TRAIL-R1, TRAIL-R2, TRAIL-R3, and/or transglutaminase.

In some embodiments, the payload protein is a cellular reprogramming factor capable of converting an at least partially differentiated cell to a less differentiated cell, such as, for example, Oct-3, Oct-4, Sox2, c-Myc, Klf4, Nanog, Lin28, ASCL1, MYT1 L, TBX3b, SV40 large T, hTERT, miR-291, miR-294, miR-295, or any combinations thereof. In some embodiments, the payload protein is a programming factor that is capable of differentiating a given cell into a desired differentiated state, such as, for example, nerve growth factor (NGF), fibroblast growth factor (FGF), interleukin-6 (IL-6), bone morphogenic protein (BMP), neurogenin3 (Ngn3), pancreatic and duodenal homeobox 1 (Pdx1), Mafa, or any combination thereof.

In some embodiments, the payload protein is a human adjuvant protein capable of eliciting an innate immune response, such as, for example, cytokines which induce or enhance an innate immune response, including IL-2, IL-12, IL-15, IL-18, IL-21 CCL21, GM-CSF and TNF-alpha; cytokines which are released from macrophages, including IL-1, IL-6, IL-8, IL-12 and TNF-alpha; from components of the complement system including C1q, MBL, C1r, C1s, C2b, Bb, D, MASP-1, MASP-2, C4b, C3b, C5a, C3a, C4a, C5b, C6, C7, C8, C9, CR1, CR2, CR3, CR4, C1qR, C1 INH, C4 bp, MCP, DAF, H, I, P and CD59; from proteins which are components of the signaling networks of the pattern recognition receptors including TLR and IL-1 R1, whereas the components are ligands of the pattern recognition receptors including IL-1 alpha, IL-1 beta, Beta-defensin, heat shock proteins, such as HSP10, HSP60, HSP65, HSP70, HSP75 and HSP90, gp96, Fibrinogen, Typlll repeat extra domain A of fibronectin; the receptors, including IL-1 RI, TLR1, TLR2, TLR3, TLR4, TLR5, TLR6, TLR7, TLR8, TLR9, TLR10, TLR11; the signal transducers including components of the Small-GTPases signaling (RhoA, Ras, Rac1, Cdc42 etc.), components of the PIP signaling (PI3K, Src-Kinases, etc.), components of the MyD88-dependent signaling (MyD88, IRAK1, IRAK2, etc.), components of the MyD88-independent signaling (TICAM1, TICAM2 etc.); activated transcription factors including e.g. NF-κB, c-Fos, c-Jun, c-Myc; and induced target genes including e.g. IL-1 alpha, IL-1 beta, Beta-Defensin, IL-6, IFN gamma, IFN alpha and IFN beta; from costimulatory molecules, including CD28 or CD40-ligand or PD1; protein domains, including LAMP; cell surface proteins; or human adjuvant proteins including CD80, CD81, CD86, trif, flt-3 ligand, thymopentin, Gp96 or fibronectin, etc., or any species homolog of any of the above human adjuvant proteins.

As described herein, the nucleotide sequence encoding the payload protein can be modified to improve expression efficiency of the protein. The methods that can be used to improve the transcription and/or translation of a gene herein are not particularly limited. For example, the nucleotide sequence can be modified to better reflect host codon usage to increase gene expression (e.g., protein production) in the host (e.g., a mammal).

The degree of payload protein expression in the cell can vary. The amount of the payload protein expressed in the subject (e.g., the serum of the subject) can vary. For example, in some embodiments the protein can be expressed in the serum of the subject in the amount of at least about 9 μg/ml, at least about 10 μg/ml, at least about 50 μg/ml, at least about 100 μg/ml, at least about 200 μg/ml, at least about 300 μg/ml, at least about 400 μg/ml, at least about 500 μg/ml, at least about 600 μg/ml, at least about 700 μg/ml, at least about 800 μg/ml, at least about 900 μg/ml, or at least about 1000 μg/ml. In some embodiments, the payload protein is expressed in the serum of the subject in the amount of about 9 μg/ml, about 10 μg/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 μg/ml, about 2000 μg/ml, about 2500 μg/ml, or a range between any two of these values. A skilled artisan will understand that the expression level in which a payload protein is needed for the method to be effective can vary depending on non-limiting factors such as the particular payload protein and the subject receiving the treatment, and an effective amount of the protein can be readily determined by a skilled artisan using conventional methods known in the art without undue experimentation.

A payload protein can be of various lengths. For example, the payload protein can be at least about 200 amino acids, at least about 250 amino acids, at least about 300 amino acids, at least about 350 amino acids, at least about 400 amino acids, at least about 450 amino acids, at least about 500 amino acids, at least about 550 amino acids, at least about 600 amino acids, at least about 650 amino acids, at least about 700 amino acids, at least about 750 amino acids, at least about 800 amino acids, or longer in length. In some embodiments, the payload protein is at least about 480 amino acids in length. In some embodiments, the payload protein is at least about 500 amino acids in length. In some embodiments, the payload protein is about 750 amino acids in length.

In some embodiments, the payload protein comprises a prodrug-converting enzyme. In some embodiments, the payload protein comprises a pro-death protein capable of halting cell growth and/or inducing cell death. The pro-death protein can be capable of halting cell growth and/or inducing cell death. The pro-death protein can comprise cytosine deaminase, thymidine kinase, Bax, Bid, Bad, Bak, BCL2L11, p53, PUMA, Diablo/SMAC, S-TRAIL, Cas9, Cas9n, hSpCas9, hSpCas9n, HSVtk, cholera toxin, diphtheria toxin, alpha toxin, anthrax toxin, exotoxin, pertussis toxin, Shiga toxin, shiga-like toxin Fas, TNF, caspase 2, caspase 3, caspase 6, caspase 7, caspase 8, caspase 9, caspase 10, caspase 11, caspase 12, purine nucleoside phosphorylase, or any combination thereof. The pro-death protein can be capable of halting cell growth and/or inducing cell death in the presence of a pro-death agent. In some embodiments, the pro-death protein is capable of halting cell growth and/or inducing cell death in the presence of a pro-death agent. Any suitable pro-death protein and pro-death agent (e.g., prodrug) is contemplated this disclosure, such as, for example, the suicide gene/prodrug combinations depicted in Table 3.

TABLE 3
PRO-DEATH PROTEINS AND PRODRUGS

| Suicide Gene | Prodrug(s) |
|---|---|
| HSV thymidine kinase (TK) | Ganciclovir (GCV); Ganciclovir elaidic acid ester; Penciclovir |
| | (PCV); Acyclovir (ACV); Valacyclovir (VCV); (E)-5-(2- |
| | bromovinyl)-2′-deoxyuridine (BVDU); Zidovuline (AZT); 2′- |
| | exo-methanocarbathymidine (MCT) |
| Cytosine Deaminase (CD) | 5-fluorocytosine (5-FC) |
| Purine nucleoside | 6-methylpurine deoxyriboside (MEP); fludarabine (FAMP) |
| phosphorylase (PNP) | |
| Cytochrome p450 enzymes | Cyclophosphamide (CPA); Ifosfamide (IFO); 4-ipomeanol (4- |
| (CYP) | IM) |
| Carboxypeptidases (CP) | 4-[(2-chloroethyl)(2-mesyloxyethyl)amino]benzoyl-L-glutamic |
| | acid (CMDA); Hydroxy-and amino-aniline mustards; |
| | Anthracycline glutamates; Methotrexate α-peptides (MTX-Phe) |
| Caspase-9 | AP1903 |
| Carboxylesterase (CE) | Irinotecan (IRT); Anthracycline acetals |
| Nitroreductase (NTR) | dinitroaziridinylbenzamide CB1954; dinitrobenzamide mustard |
| | SN23862; 4-Nitrobenzyl carbamates; Quinones |
| Horse radish peroxidase | Indole-3-acetic acid (IAA); 5-Fluoroindole-3-acetic acid (FIAA) |
| (HRP) | |
| Guanine Ribosyltransferase | 6-Thioxanthine (6-TX) |
| (XGRTP) | |
| Glycosidase enzymes | HM1826; Anthracycline acetals |
| Methionine-α,γ-lyase (MET) | Selenomethionine (SeMET) |
| Thymidine phosphorylase (TP) | 5′-Deoxy-5-fluorouridine (5′-DFU) |

The payload can be an inducer of cell death. The payload can be induce cell death by a non-endogenous cell death pathway (e.g., a bacterial pore-forming toxin). In some embodiments, the payload can be a pro-survival protein. In some embodiments, the payload is a modulator of the immune system. The payload can activate an adaptive immune response, and innate immune response, or both. In some embodiments, the cargo RNA molecule(s) encodes immunogenic material capable of stimulating an immune response (e.g., an adaptive immune response) such as, for example, antigenic peptides or proteins from a pathogen. The expression of the antigen may stimulate the body's adaptive immune system to provide an adaptive immune response. Thus, it is contemplated that some embodiments the compositions provided herein can be employed as vaccines for the prophylaxis or treatment of infectious diseases (e.g., as vaccines). The payload protein can comprise a CRE recombinase, GCaMP, a cell therapy component, a knock-down gene therapy component, a cell-surface exposed epitope, or any combination thereof.

Payloads Modulating Signaling Pathways

The receiver cell can be characterized by aberrant signaling of one or more signal transducers. In some embodiments, the aberrant signaling involves: an overactive signal transducer; a constitutively active signal transducer over a period of time; an active signal transducer repressor and an active signal transducer; an inactive signal transducer activator and an active signal transducer; an inactive signal transducer; an underactive signal transducer; a constitutively inactive signal transducer over a period of time; an inactive signal transducer repressor and an inactive signal transducer; and/or an active signal transducer activator and an inactive signal transducer. The aberrant signaling can comprise an aberrant signal of at least one signal transduction pathway regulating cell survival, cell growth, cell proliferation, cell adhesion, cell migration, cell metabolism, cell morphology, cell differentiation, apoptosis, or any combination thereof. The disease or disorder can be characterized by an aberrant signaling of the first transducer. The receiver circuit can be capable of detecting aberrant signaling, an activity of a signal transducer, an activity of a signal transducer activator and/or an activity of a signal transducer repressor. The receiver circuit can be capable of directly or indirectly inducing cell death in the presence of the aberrant signaling of one or more signal transducer(s). The receiver circuit can be capable of modulating the degree of signaling in one or more signaling pathways, thereby treating or preventing a disease or disorder. Examples of payload proteins include those associated with a signaling biochemical pathway, e.g., a signaling biochemical pathway-associated gene or polynucleotide (e.g., a signal transducer). Signal transducers can be can be associated with one or more diseases or disorders. In some embodiments, a disease or disorder is characterized by an aberrant signaling of one or more signal transducers disclosed herein. In some embodiments, the activation level of the signal transducer correlates with the occurrence and/or progression of a disease or disorder. The activation level of the signal transducer can be directly responsible or indirectly responsible for the etiology of the disease or disorder. Non-limiting examples of signal transducers, signal transduction pathways, and diseases and disorders characterized by aberrant signaling of said signal transducers are listed in Tables 4-6. In some embodiments, the methods and compositions disclosed herein prevent or treat one or more of the diseases and disorders listed in Tables 4-6. In some embodiments, the payload(s) and/or receiver circuit(s) comprises a replacement version of the signal transducer. In some embodiments, the methods and compositions further comprise knockdown of the corresponding endogenous signal transducer. The payload(s) and/or receiver circuit(s) can comprise the product of a gene listed in listed in Tables 4-6. In some embodiments, the payload(s) and/or receiver circuit(s) ameliorates a disease or disorder characterized by an aberrant signaling of one or more signaling transducers. In some embodiments, the payload(s) and/or receiver circuit(s) diminishes the activation level of one or more signal transducers (e.g., signal transducers with aberrant overactive signaling, signal transducers listed in Tables 4-6). In some embodiments, the payload(s) and/or receiver circuit(s) increases the activation level of one or more signal transducers (e.g., signal transducers with aberrant underactive signaling). In some such embodiments, the payload(s) and/or receiver circuit(s) can modulate the abundance, location, stability, and/or activity of activators or repressors of said signal transducers.

TABLE 4
DISEASES AND DISORDERS OF INTEREST

| Diseases/Disorders | Genes |
|---|---|
| Neoplasia | PTEN; ATM; ATR; EGFR; ERBB2; ERBB3; ERBB4; |
| | Notch1; Notch2; Notch3; Notch4; AKT; AKT2; AKT3; HIF; |
| | HIF1a; HIF3a; Met; HRG; Bcl2; PPAR alpha; PPAR |
| | gamma; WT1 (Wilms Tumor); FGF Receptor Family |
| | members (5 members: 1, 2, 3, 4, 5); CDKN2a; APC; RB |
| | (retinoblastoma); MEN1; VHL; BRCA1; BRCA2; AR |
| | (Androgen Receptor); TSG101; IGF; IGF Receptor; Igf1 (4 |
| | variants); Igf2 (3 variants); Igf 1 Receptor; Igf 2 Receptor; |
| | Bax; Bcl2; caspases family (9 members: |
| | 1, 2, 3, 4, 6, 7, 8, 9, 12); Kras; Apc |
| Age-related Macular | Abcr; Ccl2; Cc2; cp (ceruloplasmin); Timp3; cathepsinD; |
| Degeneration | Vldlr; Ccr2 |
| Schizophrenia | Neuregulin1 (Nrg1); Erb4 (receptor for Neuregulin); |
| | Complexin1 (Cplx1); Tph1 Tryptophan hydroxylase; Tph2 |
| | Tryptophan hydroxylase 2; Neurexin 1; GSK3; GSK3a; |
| | GSK3b |
| Disorders | 5-HTT (Slc6a4); COMT; DRD (Drd1a); SLC6A3; DAOA; |
| | DTNBP1; Dao (Dao1) |
| Trinucleotide | HTT (Huntington's Dx); SBMA/SMAX1/AR (Kennedy's |
| Repeat Disorders | Dx); FXN/X25 (Friedrich's Ataxia); ATX3 (Machado- |
| | Joseph's Dx); ATXN1 and ATXN2 (spinocerebellar |
| | ataxias); DMPK (myotonic dystrophy); Atrophin-1 and Atn1 |
| | (DRPLA Dx); CBP (Creb-BP - global instability); VLDLR |
| | (Alzheimer's); Atxn7; Atxn10 |
| Fragile X Syndrome | FMR2; FXR1; FXR2; mGLUR5 |
| Secretase Related | APH-1 (alpha and beta); Presenilin (Psen1); nicastrin |
| Disorders | (Ncstn); PEN-2 |
| Others | Nos1; Parp1; Nat1; Nat2 |
| Prion-related disorders | Prp |
| ALS | SOD1; ALS2; STEX; FUS; TARDBP; VEGF (VEGF-a; |
| | VEGF-b; VEGF-c) |
| Drug addiction | Prkce (alcohol); Drd2; Drd4; ABAT (alcohol); GRIA2; |
| | Grm5; Grin1; Htr1b; Grin2a; Drd3; Pdyn; Gria1 (alcohol) |
| Autism | Mecp2; BZRAP1; MDGA2; Sema5A; Neurexin 1; Fragile X |
| | (FMR2 (AFF2); FXR1; FXR2; Mglur5) |
| Alzheimer's Disease | E1; CHIP; UCH; UBB; Tau; LRP; PICALM; Clusterin; PS1; |
| | SORL1; CR1; Vldlr; Uba1; Uba3; CHIP28 (Aqp1, |
| | Aquaporin 1); Uchl1; Uchl3; APP |
| Inflammation | IL-10; IL-1 (IL-1a; IL-1b); IL-13; IL-17 (IL-17a (CTLA8); IL- |
| | 17b; IL-17c; IL-17d; IL-17f); II-23; Cx3cr1; ptpn22; TNFa; |
| | NOD2/CARD15 for IBD; IL-6; IL-12 (IL-12a; IL-12b); |
| | CTLA4; Cx3cl1 |
| Parkinson's Disease | x-Synuclein; DJ-1; LRRK2; Parkin; PINK1 |

TABLE 5
SIGNAL TRANSDUCERS

| | |
|---|---|
| Blood and | Anemia (CDAN1, CDA1, RPS19, DBA, PKLR, PK1, NT5C3, UMPH1, |
| coagulation diseases | PSN1, RHAG, RH50A, NRAMP2, SPTB, ALAS2, ANH1, ASB, |
| and disorders | ABCB7, ABC7, ASAT); Bare lymphocyte syndrome (TAPBP, TPSN, |
| | TAP2, ABCB3, PSF2, RING11, MHC2TA, C2TA, RFX5, RFXAP, |
| | RFX5), Bleeding disorders (TBXA2R, P2RX1, P2X1); Factor H and |
| | factor H-like 1 (HF1, CFH, HUS); Factor V and factor VIII (MCFD2); |
| | Factor VII deficiency (F7); Factor X deficiency (F10); Factor XI |
| | deficiency (F11); Factor XII deficiency (F12, HAF); Factor XIIIA |
| | deficiency (F13A1, F13A); Factor XIIIB deficiency (F13B); Fanconi |
| | anemia (FANCA, FACA, FA1, FA, FAA, FAAP95, FAAP90, FLJ34064, |
| | FANCB, FANCC, FACC, BRCA2, FANCD1, FANCD2, FANCD, |
| | FACD, FAD, FANCE, FACE, FANCF, XRCC9, FANCG, BRIP1, |
| | BACH1, FANCJ, PHF9, FANCL, FANCM, KIAA1596); |
| | Hemophagocytic lymphohistiocytosis disorders (PRF1, HPLH2, |
| | UNC13D, MUNC13-4, HPLH3, HLH3, FHL3); Hemophilia A (F8, F8C, |
| | HEMA); Hemophilia B (F9, HEMB), Hemorrhagic disorders (PI, ATT, |
| | F5); Leukocyde deficiencies and disorders (ITGB2, CD18, LCAMB, |
| | LAD, EIF2B1, EIF2BA, EIF2B2, EIF2B3, EIF2B5, LVWM, CACH, |
| | CLE, EIF2B4); Sickle cell anemia (HBB); Thalassemia (HBA2, HBB, |
| | HBD, LCRB, HBA1). |
| Cell dysregulation | B-cell non-Hodgkin lymphoma (BCL7A, BCL7); Leukemia (TAL1, |
| and oncology | TCL5, SCL, TAL2, FLT3, NBS1, NBS, ZNFN1A1, IK1, LYF1, |
| diseases and disorders | HOXD4, HOX4B, BCR, CML, PHL, ALL, ARNT, KRAS2, RASK2, |
| | GMPS, AF10, ARHGEF12, LARG, KIAA0382, CALM, CLTH, |
| | CEBPA, CEBP, CHIC2, BTL, FLT3, KIT, PBT, LPP, NPM1, NUP214, |
| | D9S46E, CAN, CAIN, RUNX1, CBFA2, AML1, WHSC1L1, NSD3, |
| | FLT3, AF1Q, NPM1, NUMA1, ZNF145, PLZF, PML, MYL, STAT5B, |
| | AF10, CALM, CLTH, ARL11, ARLTS1, P2RX7, P2X7, BCR, CML, |
| | PHL, ALL, GRAF, NF1, VRNF, WSS, NFNS, PTPN11, PTP2C, SHP2, |
| | NS1, BCL2, CCND1, PRAD1, BCL1, TCRA, GATA1, GF1, ERYF1, |
| | NFE1, ABL1, NQO1, DIA4, NMOR1, NUP214, D9S46E, CAN, CAIN). |
| Inflammation and | AIDS (KIR3DL1, NKAT3, NKB1, AMB11, KIR3DS1, IFNG, CXCL12, |
| immune related | SDF1); Autoimmune lymphoproliferative syndrome (TNFRSF6, APT1, |
| diseases and disorders | FAS, CD95, ALPS1A); Combined immunodeficiency, (IL2RG, |
| | SCIDX1, SCIDX, IMD4); HIV-1 (CCL5, SCYA5, D17S136E, TCP228), |
| | HIV susceptibility or infection (IL10, CSIF, CMKBR2, CCR2, |
| | CMKBR5, CCCKR5 (CCR5)); Immunodeficiencies (CD3E, CD3G, |
| | AICDA, AID, HIGM2, TNFRSF5, CD40, UNG, DGU, HIGM4, |
| | TNFSF5, CD40LG, HIGM1, IGM, FOXP3, IPEX, AIID, XPID, PIDX, |
| | TNFRSF14B, TACI); Inflammation (IL-10, IL-1 (IL-1a, IL-1b), IL-13, |
| | IL-17 (IL-17a (CTLA8), IL-17b, IL-17c, IL-17d, IL-17f), II-23, Cx3cr1, |
| | ptpn22, TNFa, NOD2/CARD15 for IBD, IL-6, IL-12 (IL-12a, IL-12b), |
| | CTLA4, Cx3cl1); Severe combined immunodeficiencies (SCIDs)(JAK3, |
| | JAKL, DCLRE1C, ARTEMIS, SCIDA, RAG1, RAG2, ADA, PTPRC, |
| | CD45, LCA, IL7R, CD3D, T3D, IL2RG, SCIDX1, SCIDX, IMD4). |
| Metabolic, liver, | Amyloid neuropathy (TTR, PALB); Amyloidosis (APOA1, APP, AAA, |
| kidney and protein | CVAP, AD1, GSN, FGA, LYZ, TTR, PALB); Cirrhosis (KRT18, KRT8, |
| diseases and disorders | CIRH1A, NAIC, TEX292, KIAA1988); Cystic fibrosis (CFTR, ABCC7, |
| | CF, MRP7); Glycogen storage diseases (SLC2A2, GLUT2, G6PC, |
| | G6PT, G6PT1, GAA, LAMP2, LAMPB, AGL, GDE, GBE1, GYS2, |
| | PYGL, PFKM); Hepatic adenoma (TCF1, HNF1A, MODY3), |
| | Hepatic failure, early onset, and neurologic disorder (SCOD1, SCO1), |
| | Hepatic lipase deficiency (LIPC), Hepatoblastoma, cancer and |
| | carcinomas (CTNNB1, PDGFRL, PDGRL, PRLTS, AXIN1, AXIN, |
| | CTNNB1, TP53, P53, LFS1, IGF2R, MPRI, MET, CASP8, MCH5; |
| | Medullary cystic kidney disease (UMOD, HNFJ, FJHN, MCKD2, |
| | ADMCKD2); Phenylketonuria (PAH, PKU1, QDPR, DHPR, PTS); |
| | Polycystic kidney and hepatic disease (FCYT, PKHD1, ARPKD, PKD1, |
| | PKD2, PKD4, PKDTS, PRKCSH, G19P1, PCLD, SEC63). |
| Muscular/Skeletal | Becker muscular dystrophy (DMD, BMD, MYF6), Duchenne Muscular |
| diseases and disorders | Dystrophy (DMD, BMD); Emery-Dreifuss muscular dystrophy (LMNA, |
| | LMN1, EMD2, FPLD, CMD1A, HGPS, LGMD1B, LMNA, LMN1, |
| | EMD2, FPLD, CMD1A); Facioscapulohumeral muscular dystrophy |
| | (FSHMD1A, FSHD1A); Muscular dystrophy (FKRP, MDC1C, |
| | LGMD2I, LAMA2, LAMM, LARGE, KIAA0609, MDC1D, FCMD, |
| | TTID, MYOT, CAPN3, CANP3, DYSF, LGMD2B, SGCG, LGMD2C, |
| | DMDA1, SCG3, SGCA, ADL, DAG2, LGMD2D, DMDA2, SGCB, |
| | LGMD2E, SGCD, SGD, LGMD2F, CMD1L, TCAP, LGMD2G, |
| | CMD1N, TRIM32, HT2A, LGMD2H, FKRP, MDC1C, LGMD2I, TTN, |
| | CMD1G, TMD, LGMD2J, POMT1, CAV3, LGMD1C, SEPN1, SELN, |
| | RSMD1, PLEC1, PLTN, EBS1); Osteopetrosis (LRP5, BMND1, LRP7, |
| | LR3, OPPG, VBCH2, CLCN7, CLC7, OPTA2, OSTM1, GL, TCIRG1, |
| | TIRC7, OC116, OPTB1); Muscular atrophy (VAPB, VAPC, ALS8, |
| | SMN1, SMA1, SMA2, SMA3, SMA4, BSCL2, SPG17, GARS, SMAD1, |
| | CMT2D, HEXB, IGHMBP2, SMUBP2, CATF1, SMARD1). |
| Neurological and | ALS (SOD1, ALS2, STEX, FUS, TARDBP, VEGF (VEGF-a, VEGF-b, |
| neuronal diseases | VEGF-c); Alzheimer disease (APP, AAA, CVAP, AD1, APOE, AD2, |
| and disorders | PSEN2, AD4, STM2, APBB2, FE65L1, NOS3, PLAU, URK, ACE, |
| | DCP1, ACE1, MPO, PACIP1, PAXIP1L, PTIP, A2M, BLMH, BMH, |
| | PSEN1, AD3); Autism (Mecp2, BZRAP1, MDGA2, Sema5A, Neurexin |
| | 1, GLO1, MECP2, RTT, PPMX, MRX16, MRX79, NLGN3, NLGN4, |
| | KIAA1260, AUTSX2); Fragile X Syndrome (FMR2, FXR1, FXR2, |
| | mGLUR5); Huntington's disease and disease like disorders (HD, IT15, |
| | PRNP, PRIP, JPH3, JP3, HDL2, TBP, SCA17); Parkinson disease |
| | (NR4A2, NURR1, NOT, TINUR, SNCAIP, TBP, SCA17, SNCA, |
| | NACP, PARK1, PARK4, DJ1, PARK7, LRRK2, PARK8, PINK1, |
| | PARK6, UCHL1, PARK5, SNCA, NACP, PARK1, PARK4, PRKN, |
| | PARK2, PDJ, DBH, NDUFV2); Rett syndrome (MECP2, RTT, PPMX, |
| | MRX16, MRX79, CDKL5, STK9, MECP2, RTT, PPMX, MRX16, |
| | MRX79, x-Synuclein, DJ-1); Schizophrenia (Neuregulin1 (Nrg1), Erb4 |
| | (receptor for Neuregulin), Complexin1 (Cplx1), Tph1 Tryptophan |
| | hydroxylase, Tph2, Tryptophan hydroxylase 2, Neurexin 1, GSK3, |
| | GSK3a, GSK3b, 5-HTT (Slc6a4), COMT, DRD (Drd1a), SLC6A3, |
| | DAOA, DTNBP1, Dao (Dao1)); Secretase Related Disorders (APH-1 |
| | (alpha and beta), Presenilin (Psen1), nicastrin, (Ncstn), PEN-2, Nos1, |
| | Parp1, Nat1, Nat2); Trinucleotide Repeat Disorders (HTT (Huntington's |
| | Dx), SBMA/SMAX1/AR (Kennedy's Dx), FXN/X25 (Friedrich's |
| | Ataxia), ATX3 (Machado- Joseph's Dx), ATXN1 and ATXN2 |
| | (spinocerebellar ataxias), DMPK (myotonic dystrophy), Atrophin-1 and |
| | Atn1 (DRPLA Dx), CBP (Creb-BP - global instability), VLDLR |
| | (Alzheimer's), Atxn7, Atxn10). |
| Occular diseases | Age-related macular degeneration (Abcr, Ccl2, Cc2, cp (ceruloplasmin), |
| and disorders | Timp3, cathepsinD, Vldlr, Ccr2); Cataract (CRYAA, CRYA1, CRYBB2, |
| | CRYB2, PITX3, BFSP2, CP49, CP47, CRYAA, CRYA1, PAX6, AN2, |
| | MGDA, CRYBA1, CRYB1, CRYGC, CRYG3, CCL, LIM2, MP19, |
| | CRYGD, CRYG4, BFSP2, CP49, CP47, HSF4, CTM, HSF4, CTM, |
| | MIP, AQP0, CRYAB, CRYA2, CTPP2, CRYBB1, CRYGD, CRYG4, |
| | CRYBB2, CRYB2, CRYGC, CRYG3, CCL, CRYAA, CRYA1, GJA8, |
| | CX50, CAE1, GJA3, CX46, CZP3, CAE3, CCM1, CAM, KRIT1); |
| | Corneal clouding and dystrophy (APOA1, TGFBI, CSD2, CDGG1, |
| | CSD, BIGH3, CDG2, TACSTD2, TROP2, M1S1, VSX1, RINX, PPCD, |
| | PPD, KTCN, COL8A2, FECD, PPCD2, PIP5K3, CFD); Cornea plana |
| | congenital (KERA, CNA2); Glaucoma (MYOC, TIGR, GLC1A, JOAG, |
| | GPOA, OPTN, GLC1E, FIP2, HYPL, NRP, CYP1B1, GLC3A, OPA1, |
| | NTG, NPG, CYP1B1, GLC3A); Leber congenital amaurosis (CRB1, |
| | RP12, CRX, CORD2, CRD, RPGRIP1, LCA6, CORD9, RPE65, RP20, |
| | AIPL1, LCA4, GUCY2D, GUC2D, LCA1, CORD6, RDH12, LCA3); |
| | Macular dystrophy (ELOVL4, ADMD, STGD2, STGD3, RDS, RP7, |
| | PRPH2, PRPH, AVMD, AOFMD, VMD2). |

TABLE 6
SIGNAL TRANSDUCTION PATHWAYS

| Pathway | Gene(s) |
|---|---|
| PI3K/AKT Signaling | PRKCE; ITGAM; ITGA5; IRAK1; PRKAA2; EIF2AK2; |
| | PTEN; EIF4E; PRKCZ; GRK6; MAPK1; TSC1; PLK1; |
| | AKT2; IKBKB; PIK3CA; CDK8; CDKN1B; NFKB2; BCL2; |
| | PIK3CB; PPP2R1A; MAPK8; BCL2L1; MAPK3; TSC2; |
| | ITGA1; KRAS; EIF4EBP1; RELA; PRKCD; NOS3; |
| | PRKAA1; MAPK9; CDK2; PPP2CA; PIM1; ITGB7; |
| | YWHAZ; ILK; TP53; RAF1; IKBKG; RELB; DYRK1A; |
| | CDKN1A; ITGB1; MAP2K2; JAK1; AKT1; JAK2; PIK3R1; |
| | CHUK; PDPK1; PPP2R5C; CTNNB1; MAP2K1; NFKB1; |
| | PAK3; ITGB3; CCND1; GSK3A; FRAP1; SFN; ITGA2; |
| | TTK; CSNK1A1; BRAF; GSK3B; AKT3; FOXO1; SGK; |
| | HSP90AA1; RPS6KB1 |
| ERK/MAPK Signaling | PRKCE; ITGAM; ITGA5; HSPB1; IRAK1; PRKAA2; |
| | EIF2AK2; RAC1; RAP1A; TLN1; EIF4E; ELK1; GRK6; |
| | MAPK1; RAC2; PLK1; AKT2; PIK3CA; CDK8; CREB1; |
| | PRKCI; PTK2; FOS; RPS6KA4; PIK3CB; PPP2R1A; |
| | PIK3C3; MAPK8; MAPK3; ITGA1; ETS1; KRAS; MYCN; |
| | EIF4EBP1; PPARG; PRKCD; PRKAA1; MAPK9; SRC; |
| | CDK2; PPP2CA; PIM1; PIK3C2A; ITGB7; YWHAZ; |
| | PPP1CC; KSR1; PXN; RAF1; FYN; DYRK1A; ITGB1; |
| | MAP2K2; PAK4; PIK3R1; STAT3; PPP2R5C; MAP2K1; |
| | PAK3; ITGB3; ESR1; ITGA2; MYC; TTK; CSNK1A1; |
| | CRKL; BRAF; ATF4; PRKCA; SRF; STAT1; SGK |
| Glucocorticoid Receptor | RAC1; TAF4B; EP300; SMAD2; TRAF6; PCAF; ELK1; |
| Signaling | MAPK1; SMAD3; AKT2; IKBKB; NCOR2; UBE2I; |
| | PIK3CA; CREB1; FOS; HSPA5; NFKB2; BCL2; |
| | MAP3K14; STAT5B; PIK3CB; PIK3C3; MAPK8; BCL2L1; |
| | MAPK3; TSC22D3; MAPK10; NRIP1; KRAS; MAPK13; |
| | RELA; STAT5A; MAPK9; NOS2A; PBX1; NR3C1; |
| | PIK3C2A; CDKN1C; TRAF2; SERPINE1; NCOA3; |
| | MAPK14; TNF; RAF1; IKBKG; MAP3K7; CREBBP; |
| | CDKN1A; MAP2K2; JAK1; IL8; NCOA2; AKT1; JAK2; |
| | PIK3R1; CHUK; STAT3; MAP2K1; NFKB1; TGFBR1; |
| | ESR1; SMAD4; CEBPB; JUN; AR; AKT3; CCL2; MMP1; |
| | STAT1; IL6; HSP90AA1 |
| Axonal Guidance Signaling | PRKCE; ITGAM; ROCK1; ITGA5; CXCR4; ADAM12; |
| | IGF1; RAC1; RAP1A; EIF4E; PRKCZ; NRP1; NTRK2; |
| | ARHGEF7; SMO; ROCK2; MAPK1; PGF; RAC2; |
| | PTPN11; GNAS; AKT2; PIK3CA; ERBB2; PRKCI; PTK2; |
| | CFL1; GNAQ; PIK3CB; CXCL12; PIK3C3; WNT11; |
| | PRKD1; GNB2L1; ABL1; MAPK3; ITGA1; KRAS; RHOA; |
| | PRKCD; PIK3C2A; ITGB7; GLI2; PXN; VASP; RAF1; |
| | FYN; ITGB1; MAP2K2; PAK4; ADAM17; AKT1; PIK3R1; |
| | GLI1; WNT5A; ADAM10; MAP2K1; PAK3; ITGB3; |
| | CDC42; VEGFA; ITGA2; EPHA8; CRKL; RND1; GSK3B; |
| | AKT3; PRKCA |
| Ephrin Receptor Signaling | PRKCE; ITGAM; ROCK1; ITGA5; CXCR4; IRAK1; |
| | PRKAA2; EIF2AK2; RAC1; RAP1A; GRK6; ROCK2; |
| | MAPK1; PGF; RAC2; PTPN11; GNAS; PLK1; AKT2; |
| | DOK1; CDK8; CREB1; PTK2; CFL1; GNAQ; MAP3K14; |
| | CXCL12; MAPK8; GNB2L1; ABL1; MAPK3; ITGA1; |
| | KRAS; RHOA; PRKCD; PRKAA1; MAPK9; SRC; CDK2; |
| | PIM1; ITGB7; PXN; RAF1; FYN; DYRK1A; ITGB1; |
| | MAP2K2; PAK4; AKT1; JAK2; STAT3; ADAM10; |
| | MAP2K1; PAK3; ITGB3; CDC42; VEGFA; ITGA2; |
| | EPHA8; TTK; CSNK1A1; CRKL; BRAF; PTPN13; ATF4; |
| | AKT3; SGK |
| Actin Cytoskeleton | ACTN4; PRKCE; ITGAM; ROCK1; ITGA5; IRAK1; |
| Signaling | PRKAA2; EIF2AK2; RAC1; INS; ARHGEF7; GRK6; |
| | ROCK2; MAPK1; RAC2; PLK1; AKT2; PIK3CA; CDK8; |
| | PTK2; CFL1; PIK3CB; MYH9; DIAPH1; PIK3C3; MAPK8; |
| | F2R; MAPK3; SLC9A1; ITGA1; KRAS; RHOA; PRKCD; |
| | PRKAA1; MAPK9; CDK2; PIM1; PIK3C2A; ITGB7; |
| | PPP1CC; PXN; VIL2; RAF1; GSN; DYRK1A; ITGB1; |
| | MAP2K2; PAK4; PIP5K1A; PIK3R1; MAP2K1; PAK3; |
| | ITGB3; CDC42; APC; ITGA2; TTK; CSNK1A1; CRKL; |
| | BRAF; VAV3; SGK |
| Huntington's Disease | PRKCE; IGF1; EP300; RCOR1; PRKCZ; HDAC4; TGM2; |
| Signaling | MAPK1; CAPNS1; AKT2; EGFR; NCOR2; SP1; CAPN2; |
| | PIK3CA; HDAC5; CREB1; PRKCI; HSPA5; REST; |
| | GNAQ; PIK3CB; PIK3C3; MAPK8; IGF1R; PRKD1; |
| | GNB2L1; BCL2L1; CAPN1; MAPK3; CASP8; HDAC2; |
| | HDAC7A; PRKCD; HDAC11; MAPK9; HDAC9; PIK3C2A; |
| | HDAC3; TP53; CASP9; CREBBP; AKT1; PIK3R1; |
| | PDPK1; CASP1; APAF1; FRAP1; CASP2; JUN; BAX; |
| | ATF4; AKT3; PRKCA; CLTC; SGK; HDAC6; CASP3 |
| Apoptosis Signaling | PRKCE; ROCK1; BID; IRAK1; PRKAA2; EIF2AK2; BAK1; |
| | BIRC4; GRK6; MAPK1; CAPNS1; PLK1; AKT2; IKBKB; |
| | CAPN2; CDK8; FAS; NFKB2; BCL2; MAP3K14; MAPK8; |
| | BCL2L1; CAPN1; MAPK3; CASP8; KRAS; RELA; |
| | PRKCD; PRKAA1; MAPK9; CDK2; PIM1; TP53; TNF; |
| | RAF1; IKBKG; RELB; CASP9; DYRK1A; MAP2K2; |
| | CHUK; APAF1; MAP2K1; NFKB1; PAK3; LMNA; CASP2; |
| | BIRC2; TTK; CSNK1A1; BRAF; BAX; PRKCA; SGK; |
| | CASP3; BIRC3; PARP1 |
| B Cell Receptor Signaling | RAC1; PTEN; LYN; ELK1; MAPK1; RAC2; PTPN11; |
| | AKT2; IKBKB; PIK3CA; CREB1; SYK; NFKB2; CAMK2A; |
| | MAP3K14; PIK3CB; PIK3C3; MAPK8; BCL2L1; ABL1; |
| | MAPK3; ETS1; KRAS; MAPK13; RELA; PTPN6; MAPK9; |
| | EGR1; PIK3C2A; BTK; MAPK14; RAF1; IKBKG; RELB; |
| | MAP3K7; MAP2K2; AKT1; PIK3R1; CHUK; MAP2K1; |
| | NFKB1; CDC42; GSK3A; FRAP1; BCL6; BCL10; JUN; |
| | GSK3B; ATF4; AKT3; VAV3; RPS6KB1 |
| Leukocyte Extravasation | ACTN4; CD44; PRKCE; ITGAM; ROCK1; CXCR4; CYBA; |
| Signaling | RAC1; RAP1A; PRKCZ; ROCK2; RAC2; PTPN11; |
| | MMP14; PIK3CA; PRKCI; PTK2; PIK3CB; CXCL12; |
| | PIK3C3; MAPK8; PRKD1; ABL1; MAPK10; CYBB; |
| | MAPK13; RHOA; PRKCD; MAPK9; SRC; PIK3C2A; BTK; |
| | MAPK14; NOX1; PXN; VIL2; VASP; ITGB1; MAP2K2; |
| | CTNND1; PIK3R1; CTNNB1; CLDN1; CDC42; F11R; ITK; |
| | CRKL; VAV3; CTTN; PRKCA; MMP1; MMP9 |
| Integrin Signaling | ACTN4; ITGAM; ROCK1; ITGA5; RAC1; PTEN; RAP1A; |
| | TLN1; ARHGEF7; MAPK1; RAC2; CAPNS1; AKT2; |
| | CAPN2; PIK3CA; PTK2; PIK3CB; PIK3C3; MAPK8; |
| | CAV1; CAPN1; ABL1; MAPK3; ITGA1; KRAS; RHOA; |
| | SRC; PIK3C2A; ITGB7; PPP1CC; ILK; PXN; VASP; |
| | RAF1; FYN; ITGB1; MAP2K2; PAK4; AKT1; PIK3R1; |
| | TNK2; MAP2K1; PAK3; ITGB3; CDC42; RND3; ITGA2; |
| | CRKL; BRAF; GSK3B; AKT3 |
| Acute Phase Response | IRAK1; SOD2; MYD88; TRAF6; ELK1; MAPK1; PTPN11; |
| Signaling | AKT2; IKBKB; PIK3CA; FOS; NFKB2; MAP3K14; |
| | PIK3CB; MAPK8; RIPK1; MAPK3; IL6ST; KRAS; |
| | MAPK13; IL6R; RELA; SOCS1; MAPK9; FTL; NR3C1; |
| | TRAF2; SERPINE1; MAPK14; TNF; RAF1; PDK1; |
| | IKBKG; RELB; MAP3K7; MAP2K2; AKT1; JAK2; PIK3R1; |
| | CHUK; STAT3; MAP2K1; NFKB1; FRAP1; CEBPB; JUN; |
| | AKT3; IL1R1; IL6 |
| PTEN Signaling | ITGAM; ITGA5; RAC1; PTEN; PRKCZ; BCL2L11; |
| | MAPK1; RAC2; AKT2; EGFR; IKBKB; CBL; PIK3CA; |
| | CDKN1B; PTK2; NFKB2; BCL2; PIK3CB; BCL2L1; |
| | MAPK3; ITGA1; KRAS; ITGB7; ILK; PDGFRB; INSR; |
| | RAF1; IKBKG; CASP9; CDKN1A; ITGB1; MAP2K2; |
| | AKT1; PIK3R1; CHUK; PDGFRA; PDPK1; MAP2K1; |
| | NFKB1; ITGB3; CDC42; CCND1; GSK3A; ITGA2; |
| | GSK3B; AKT3; FOXO1; CASP3; RPS6KB1 |
| p53 Signaling | PTEN; EP300; BBC3; PCAF; FASN; BRCA1; GADD45A; |
| | BIRC5; AKT2; PIK3CA; CHEK1; TP53INP1; BCL2; |
| | PIK3CB; PIK3C3; MAPK8; THBS1; ATR; BCL2L1; E2F1; |
| | PMAIP1; CHEK2; TNFRSF10B; TP73; RB1; HDAC9; |
| | CDK2; PIK3C2A; MAPK14; TP53; LRDD; CDKN1A; |
| | HIPK2; AKT1; PIK3R1; RRM2B; APAF1; CTNNB1; |
| | SIRT1; CCND1; PRKDC; ATM; SFN; CDKN2A; JUN; |
| | SNAI2; GSK3B; BAX; AKT3 |
| Aryl Hydrocarbon Receptor | HSPB1; EP300; FASN; TGM2; RXRA; MAPK1; NQO1; |
| Signaling | NCOR2; SP1; ARNT; CDKN1B; FOS; CHEK1; |
| | SMARCA4; NFKB2; MAPK8; ALDH1A1; ATR; E2F1; |
| | MAPK3; NRIP1; CHEK2; RELA; TP73; GSTP1; RB1; |
| | SRC; CDK2; AHR; NFE2L2; NCOA3; TP53; TNF; |
| | CDKN1A; NCOA2; APAF1; NFKB1; CCND1; ATM; ESR1; |
| | CDKN2A; MYC; JUN; ESR2; BAX; IL6; CYP1B1; |
| | HSP90AA1 |
| Xenobiotic Metabolism | PRKCE; EP300; PRKCZ; RXRA; MAPK1; NQO1; |
| Signaling | NCOR2; PIK3CA; ARNT; PRKCI; NFKB2; CAMK2A; |
| | PIK3CB; PPP2R1A; PIK3C3; MAPK8; PRKD1; |
| | ALDH1A1; MAPK3; NRIP1; KRAS; MAPK13; PRKCD; |
| | GSTP1; MAPK9; NOS2A; ABCB1; AHR; PPP2CA; FTL; |
| | NFE2L2; PIK3C2A; PPARGC1A; MAPK14; TNF; RAF1; |
| | CREBBP; MAP2K2; PIK3R1; PPP2R5C; MAP2K1; |
| | NFKB1; KEAP1; PRKCA; EIF2AK3; IL6; CYP1B1; |
| | HSP90AA1 |
| SAPK/JNK Signaling | PRKCE; IRAK1; PRKAA2; EIF2AK2; RAC1; ELK1; |
| | GRK6; MAPK1; GADD45A; RAC2; PLK1; AKT2; PIK3CA; |
| | FADD; CDK8; PIK3CB; PIK3C3; MAPK8; RIPK1; |
| | GNB2L1; IRS1; MAPK3; MAPK10; DAXX; KRAS; |
| | PRKCD; PRKAA1; MAPK9; CDK2; PIM1; PIK3C2A; |
| | TRAF2; TP53; LCK; MAP3K7; DYRK1A; MAP2K2; |
| | PIK3R1; MAP2K1; PAK3; CDC42; JUN; TTK; CSNK1A1; |
| | CRKL; BRAF; SGK |
| PPAr/RXR Signaling | PRKAA2; EP300; INS; SMAD2; TRAF6; PPARA; FASN; |
| | RXRA; MAPK1; SMAD3; GNAS; IKBKB; NCOR2; |
| | ABCA1; GNAQ; NFKB2; MAP3K14; STAT5B; MAPK8; |
| | IRS1; MAPK3; KRAS; RELA; PRKAA1; PPARGC1A; |
| | NCOA3; MAPK14; INSR; RAF1; IKBKG; RELB; MAP3K7; |
| | CREBBP; MAP2K2; JAK2; CHUK; MAP2K1; NFKB1; |
| | TGFBR1; SMAD4; JUN; IL1R1; PRKCA; IL6; HSP90AA1; |
| | ADIPOQ |
| NF-KB Signaling | IRAK1; EIF2AK2; EP300; INS; MYD88; PRKCZ; TRAF6; |
| | TBK1; AKT2; EGFR; IKBKB; PIK3CA; BTRC; NFKB2; |
| | MAP3K14; PIK3CB; PIK3C3; MAPK8; RIPK1; HDAC2; |
| | KRAS; RELA; PIK3C2A; TRAF2; TLR4; PDGFRB; TNF; |
| | INSR; LCK; IKBKG; RELB; MAP3K7; CREBBP; AKT1; |
| | PIK3R1; CHUK; PDGFRA; NFKB1; TLR2; BCL10; |
| | GSK3B; AKT3; TNFAIP3; IL1R1 |
| Neuregulin Signaling | ERBB4; PRKCE; ITGAM; ITGA5; PTEN; PRKCZ; ELK1; |
| | MAPK1; PTPN11; AKT2; EGFR; ERBB2; PRKCI; |
| | CDKN1B; STAT5B; PRKD1; MAPK3; ITGA1; KRAS; |
| | PRKCD; STAT5A; SRC; ITGB7; RAF1; ITGB1; MAP2K2; |
| | ADAM17; AKT1; PIK3R1; PDPK1; MAP2K1; ITGB3; |
| | EREG; FRAP1; PSEN1; ITGA2; MYC; NRG1; CRKL; |
| | AKT3; PRKCA; HSP90AA1; RPS6KB1 |
| Wnt & Beta catenin | CD44; EP300; LRP6; DVL3; CSNK1E; GJA1; SMO; |
| Signaling | AKT2; PIN1; CDH1; BTRC; GNAQ; MARK2; PPP2R1A; |
| | WNT11; SRC; DKK1; PPP2CA; SOX6; SFRP2; ILK; |
| | LEF1; SOX9; TP53; MAP3K7; CREBBP; TCF7L2; AKT1; |
| | PPP2R5C; WNT5A; LRP5; CTNNB1; TGFBR1; CCND1; |
| | GSK3A; DVL1; APC; CDKN2A; MYC; CSNK1A1; GSK3B; |
| | AKT3; SOX2 |
| Insulin Receptor Signaling | PTEN; INS; EIF4E; PTPN1; PRKCZ; MAPK1; TSC1; |
| | PTPN11; AKT2; CBL; PIK3CA; PRKCI; PIK3CB; PIK3C3; |
| | MAPK8; IRS1; MAPK3; TSC2; KRAS; EIF4EBP1; |
| | SLC2A4; PIK3C2A; PPP1CC; INSR; RAF1; FYN; |
| | MAP2K2; JAK1; AKT1; JAK2; PIK3R1; PDPK1; MAP2K1; |
| | GSK3A; FRAP1; CRKL; GSK3B; AKT3; FOXO1; SGK; |
| | RPS6KB1 |
| IL-6 Signaling | HSPB1; TRAF6; MAPKAPK2; ELK1; MAPK1; PTPN11; |
| | IKBKB; FOS; NFKB2; MAP3K14; MAPK8; MAPK3; |
| | MAPK10; IL6ST; KRAS; MAPK13; IL6R; RELA; SOCS1; |
| | MAPK9; ABCB1; TRAF2; MAPK14; TNF; RAF1; IKBKG; |
| | RELB; MAP3K7; MAP2K2; IL8; JAK2; CHUK; STAT3; |
| | MAP2K1; NFKB1; CEBPB; JUN; IL1R1; SRF; IL6 |
| Hepatic Cholestasis | PRKCE; IRAK1; INS; MYD88; PRKCZ; TRAF6; PPARA; |
| | RXRA; IKBKB; PRKCI; NFKB2; MAP3K14; MAPK8; |
| | PRKD1; MAPK10; RELA; PRKCD; MAPK9; ABCB1; |
| | TRAF2; TLR4; TNF; INSR; IKBKG; RELB; MAP3K7; IL8; |
| | CHUK; NR1H2; TJP2; NFKB1; ESR1; SREBF1; FGFR4; |
| | JUN; IL1R1; PRKCA; IL6 |
| IGF-1 Signaling | IGF1; PRKCZ; ELK1; MAPK1; PTPN11; NEDD4; AKT2; |
| | PIK3CA; PRKCI; PTK2; FOS; PIK3CB; PIK3C3; MAPK8; |
| | IGF1R; IRS1; MAPK3; IGFBP7; KRAS; PIK3C2A; |
| | YWHAZ; PXN; RAF1; CASP9; MAP2K2; AKT1; PIK3R1; |
| | PDPK1; MAP2K1; IGFBP2; SFN; JUN; CYR61; AKT3; |
| | FOXO1; SRF; CTGF; RPS6KB1 |
| NRF2-mediated Oxidative | PRKCE; EP300; SOD2; PRKCZ; MAPK1; SQSTM1; |
| Stress Response | NQO1; PIK3CA; PRKCI; FOS; PIK3CB; PIK3C3; MAPK8; |
| | PRKD1; MAPK3; KRAS; PRKCD; GSTP1; MAPK9; FTL; |
| | NFE2L2; PIK3C2A; MAPK14; RAF1; MAP3K7; CREBBP; |
| | MAP2K2; AKT1; PIK3R1; MAP2K1; PPIB; JUN; KEAP1; |
| | GSK3B; ATF4; PRKCA; EIF2AK3; HSP90AA1 |
| Hepatic Fibrosis/Hepatic | EDN1; IGF1; KDR; FLT1; SMAD2; FGFR1; MET; PGF; |
| Stellate Cell Activation | SMAD3; EGFR; FAS; CSF1; NFKB2; BCL2; MYH9; |
| | IGF1R; IL6R; RELA; TLR4; PDGFRB; TNF; RELB; IL8; |
| | PDGFRA; NFKB1; TGFBR1; SMAD4; VEGFA; BAX; |
| | IL1R1; CCL2; HGF; MMP1; STAT1; IL6; CTGF; MMP9 |
| PPAR Signaling | EP300; INS; TRAF6; PPARA; RXRA; MAPK1; IKBKB; |
| | NCOR2; FOS; NFKB2; MAP3K14; STAT5B; MAPK3; |
| | NRIP1; KRAS; PPARG; RELA; STAT5A; TRAF2; |
| | PPARGC1A; PDGFRB; TNF; INSR; RAF1; IKBKG; |
| | RELB; MAP3K7; CREBBP; MAP2K2; CHUK; PDGFRA; |
| | MAP2K1; NFKB1; JUN; IL1R1; HSP90AA1 |
| Fc Epsilon RI Signaling | PRKCE; RAC1; PRKCZ; LYN; MAPK1; RAC2; PTPN11; |
| | AKT2; PIK3CA; SYK; PRKCI; PIK3CB; PIK3C3; MAPK8; |
| | PRKD1; MAPK3; MAPK10; KRAS; MAPK13; PRKCD; |
| | MAPK9; PIK3C2A; BTK; MAPK14; TNF; RAF1; FYN; |
| | MAP2K2; AKT1; PIK3R1; PDPK1; MAP2K1; AKT3; |
| | VAV3; PRKCA |
| G-Protein Coupled | PRKCE; RAP1A; RGS16; MAPK1; GNAS; AKT2; IKBKB; |
| Receptor Signaling | PIK3CA; CREB1; GNAQ; NFKB2; CAMK2A; PIK3CB; |
| | PIK3C3; MAPK3; KRAS; RELA; SRC; PIK3C2A; RAF1; |
| | IKBKG; RELB; FYN; MAP2K2; AKT1; PIK3R1; CHUK; |
| | PDPK1; STAT3; MAP2K1; NFKB1; BRAF; ATF4; AKT3; |
| | PRKCA |
| Inositol Phosphate | PRKCE; IRAK1; PRKAA2; EIF2AK2; PTEN; GRK6; |
| Metabolism | MAPK1; PLK1; AKT2; PIK3CA; CDK8; PIK3CB; PIK3C3; |
| | MAPK8; MAPK3; PRKCD; PRKAA1; MAPK9; CDK2; |
| | PIM1; PIK3C2A; DYRK1A; MAP2K2; PIP5K1A; PIK3R1; |
| | MAP2K1; PAK3; ATM; TTK; CSNK1A1; BRAF; SGK |
| PDGF Signaling | EIF2AK2; ELK1; ABL2; MAPK1; PIK3CA; FOS; PIK3CB; |
| | PIK3C3; MAPK8; CAV1; ABL1; MAPK3; KRAS; SRC; |
| | PIK3C2A; PDGFRB; RAF1; MAP2K2; JAK1; JAK2; |
| | PIK3R1; PDGFRA; STAT3; SPHK1; MAP2K1; MYC; |
| | JUN; CRKL; PRKCA; SRF; STAT1; SPHK2 |
| VEGF Signaling | ACTN4; ROCK1; KDR; FLT1; ROCK2; MAPK1; PGF; |
| | AKT2; PIK3CA; ARNT; PTK2; BCL2; PIK3CB; PIK3C3; |
| | BCL2L1; MAPK3; KRAS; HIF1A; NOS3; PIK3C2A; PXN; |
| | RAF1; MAP2K2; ELAVL1; AKT1; PIK3R1; MAP2K1; SFN; |
| | VEGFA; AKT3; FOXO1; PRKCA |
| Natural Killer Cell Signaling | PRKCE; RAC1; PRKCZ; MAPK1; RAC2; PTPN11; |
| | KIR2DL3; AKT2; PIK3CA; SYK; PRKCI; PIK3CB; |
| | PIK3C3; PRKD1; MAPK3; KRAS; PRKCD; PTPN6; |
| | PIK3C2A; LCK; RAF1; FYN; MAP2K2; PAK4; AKT1; |
| | PIK3R1; MAP2K1; PAK3; AKT3; VAV3; PRKCA |
| Cell Cycle: G1/S | HDAC4; SMAD3; SUV39H1; HDAC5; CDKN1B; BTRC; |
| Checkpoint Regulation | ATR; ABL1; E2F1; HDAC2; HDAC7A; RB1; HDAC11; |
| | HDAC9; CDK2; E2F2; HDAC3; TP53; CDKN1A; CCND1; |
| | E2F4; ATM; RBL2; SMAD4; CDKN2A; MYC; NRG1; |
| | GSK3B; RBL1; HDAC6 |
| T Cell Receptor Signaling | RAC1; ELK1; MAPK1; IKBKB; CBL; PIK3CA; FOS; |
| | NFKB2; PIK3CB; PIK3C3; MAPK8; MAPK3; KRAS; |
| | RELA; PIK3C2A; BTK; LCK; RAF1; IKBKG; RELB; FYN; |
| | MAP2K2; PIK3R1; CHUK; MAP2K1; NFKB1; ITK; BCL10; |
| | JUN; VAV3 |
| Death Receptor Signaling | CRADD; HSPB1; BID; BIRC4; TBK1; IKBKB; FADD; |
| | FAS; NFKB2; BCL2; MAP3K14; MAPK8; RIPK1; CASP8; |
| | DAXX; TNFRSF10B; RELA; TRAF2; TNF; IKBKG; RELB; |
| | CASP9; CHUK; APAF1; NFKB1; CASP2; BIRC2; CASP3; |
| | BIRC3 |
| FGF Signaling | RAC1; FGFR1; MET; MAPKAPK2; MAPK1; PTPN11; |
| | AKT2; PIK3CA; CREB1; PIK3CB; PIK3C3; MAPK8; |
| | MAPK3; MAPK13; PTPN6; PIK3C2A; MAPK14; RAF1; |
| | AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; |
| | STAT5B; PIK3CB; PIK3C3; GNB2L1; BCL2L1; MAPK3; |
| | ETS1; KRAS; RUNX1; PIM1; PIK3C2A; RAF1; MAP2K2; |
| | AKT1; JAK2; PIK3R1; STAT3; MAP2K1; CCND1; AKT3; |
| | STAT1 |
| Amyotrophic Lateral | BID; IGF1; RAC1; BIRC4; PGF; CAPNS1; CAPN2; |
| Sclerosis Signaling | PIK3CA; BCL2; PIK3CB; PIK3C3; BCL2L1; CAPN1; |
| | PIK3C2A; TP53; CASP9; PIK3R1; RAB5A; CASP1; |
| | APAF1; VEGFA; BIRC2; BAχ; AKT3; CASP3; BIRC3 |
| JAK/Stat Signaling | PTPN1; MAPK1; PTPN11; AKT2; PIK3CA; STAT5B; |
| | PIK3CB; PIK3C3; MAPK3; KRAS; SOCS1; STAT5A; |
| | PTPN6; PIK3C2A; RAF1; CDKN1A; MAP2K2; JAK1; |
| | AKT1; JAK2; PIK3R1; STAT3; MAP2K1; FRAP1; AKT3; |
| | STAT1 |
| Nicotinate and Nicotinamide | PRKCE; IRAK1; PRKAA2; EIF2AK2; GRK6; MAPK1; |
| Metabolism | PLK1; AKT2; CDK8; MAPK8; MAPK3; PRKCD; PRKAA1; |
| | PBEF1; MAPK9; CDK2; PIM1; DYRK1A; MAP2K2; |
| | MAP2K1; PAK3; NT5E; TTK; CSNK1A1; BRAF; SGK |
| Chemokine Signaling | CXCR4; ROCK2; MAPK1; PTK2; FOS; CFL1; GNAQ; |
| | CAMK2A; CXCL12; MAPK8; MAPK3; KRAS; MAPK13; |
| | RHOA; CCR3; SRC; PPP1CC; MAPK14; NOX1; RAF1; |
| | MAP2K2; MAP2K1; JUN; CCL2; PRKCA |
| IL-2 Signaling | ELK1; MAPK1; PTPN11; AKT2; PIK3CA; SYK; FOS; |
| | STAT5B; PIK3CB; PIK3C3; MAPK8; MAPK3; KRAS; |
| | SOCS1; STAT5A; PIK3C2A; LCK; RAF1; MAP2K2; |
| | JAK1; AKT1; PIK3R1; MAP2K1; JUN; AKT3 |
| Synaptic Long Term | PRKCE; IGF1; PRKCZ; PRDX6; LYN; MAPK1; GNAS; |
| Depression | PRKCI; GNAQ; PPP2R1A; IGF1R; PRKD1; MAPK3; |
| | KRAS; GRN; PRKCD; NOS3; NOS2A; PPP2CA; |
| | YWHAZ; RAF1; MAP2K2; PPP2R5C; MAP2K1; PRKCA |
| Estrogen Receptor | TAF4B; EP300; CARM1; PCAF; MAPK1; NCOR2; |
| Signaling | SMARCA4; MAPK3; NRIP1; KRAS; SRC; NR3C1; |
| | HDAC3; PPARGC1A; RBM9; NCOA3; RAF1; CREBBP; |
| | MAP2K2; NCOA2; MAP2K1; PRKDC; ESR1; ESR2 |
| Protein Ubiquitination | TRAF6; SMURF1; BIRC4; BRCA1; UCHL1; NEDD4; |
| Pathway | CBL; UBE2I; BTRC; HSPA5; USP7; USP10; FBXW7; |
| | USP9X; STUB1; USP22; B2M; BIRC2; PARK2; USP8; |
| | USP1; VHL; HSP90AA1; BIRC3 |
| IL-10 Signaling | TRAF6; CCR1; ELK1; IKBKB; SP1; FOS; NFKB2; |
| | MAP3K14; MAPK8; MAPK13; RELA; MAPK14; TNF; |
| | IKBKG; RELB; MAP3K7; JAK1; CHUK; STAT3; NFKB1; |
| | JUN; IL1R1; IL6 |
| VDR/RXR Activation | PRKCE; EP300; PRKCZ; RXRA; GADD45A; HES1; |
| | NCOR2; SP1; PRKCI; CDKN1B; PRKD1; PRKCD; |
| | RUNX2; KLF4; YY1; NCOA3; CDKN1A; NCOA2; SPP1; |
| | LRP5; CEBPB; FOXO1; PRKCA |
| TGF-beta Signaling | EP300; SMAD2; SMURF1; MAPK1; SMAD3; SMAD1; |
| | FOS; MAPK8; MAPK3; KRAS; MAPK9; RUNX2; |
| | SERPINE1; RAF1; MAP3K7; CREBBP; MAP2K2; |
| | MAP2K1; TGFBR1; SMAD4; JUN; SMAD5 |
| Toll-like Receptor Signaling | IRAK1; EIF2AK2; MYD88; TRAF6; PPARA; ELK1; |
| | IKBKB; FOS; NFKB2; MAP3K14; MAPK8; MAPK13; |
| | RELA; TLR4; MAPK14; IKBKG; RELB; MAP3K7; CHUK; |
| | NFKB1; TLR2; JUN |
| p38 MAPK Signaling | HSPB1; IRAK1; TRAF6; MAPKAPK2; ELK1; FADD; FAS; |
| | CREB1; DDIT3; RPS6KA4; DAXX; MAPK13; TRAF2; |
| | MAPK14; TNF; MAP3K7; TGFBR1; MYC; ATF4; IL1R1; |
| | SRF; STAT1 |
| Neurotrophin/TRK Signaling | NTRK2; MAPK1; PTPN11; PIK3CA; CREB1; FOS; |
| | PIK3CB; PIK3C3; MAPK8; MAPK3; KRAS; PIK3C2A; |
| | RAF1; MAP2K2; AKT1; PIK3R1; PDPK1; MAP2K1; |
| | CDC42; JUN; ATF4 |
| FXR/RXR Activation | INS; PPARA; FASN; RXRA; AKT2; SDC1; MAPK8; |
| | APOB; MAPK10; PPARG; MTTP; MAPK9; PPARGC1A; |
| | TNF; CREBBP; AKT1; SREBF1; FGFR4; AKT3; FOXO1 |
| Synaptic Long Term | PRKCE; RAP1A; EP300; PRKCZ; MAPK1; CREB1; |
| Potentiation | PRKCI; GNAQ; CAMK2A; PRKD1; MAPK3; KRAS; |
| | PRKCD; PPP1CC; RAF1; CREBBP; MAP2K2; MAP2K1; |
| | ATF4; PRKCA |
| Calcium Signaling | RAP1A; EP300; HDAC4; MAPK1; HDAC5; CREB1; |
| | CAMK2A; MYH9; MAPK3; HDAC2; HDAC7A; HDAC11; |
| | HDAC9; HDAC3; CREBBP; CALR; CAMKK2; ATF4; |
| | HDAC6 |
| EGF Signaling | ELK1; MAPK1; EGFR; PIK3CA; FOS; PIK3CB; PIK3C3; |
| | MAPK8; MAPK3; PIK3C2A; RAF1; JAK1; PIK3R1; |
| | STAT3; MAP2K1; JUN; PRKCA; SRF; STAT1 |
| Hypoxia Signaling in the | EDN1; PTEN; EP300; NQO1; UBE2I; CREB1; ARNT; |
| Cardiovascular System | HIF1A; SLC2A4; NOS3; TP53; LDHA; AKT1; ATM; |
| | VEGFA; JUN; ATF4; VHL; HSP90AA1 |
| LPS/IL-1 Mediated Inhibition | IRAK1; MYD88; TRAF6; PPARA; RXRA; ABCA1; |
| of RXR Function | MAPK8; ALDH1A1; GSTP1; MAPK9; ABCB1; TRAF2; |
| | TLR4; TNF; MAP3K7; NR1H2; SREBF1; JUN; IL1R1 |
| LXR/RXR Activation | FASN; RXRA; NCOR2; ABCA1; NFKB2; IRF3; RELA; |
| | NOS2A; TLR4; TNF; RELB; LDLR; NR1H2; NFKB1; |
| | SREBF1; IL1R1; CCL2; IL6; MMP9 |
| Amyloid Processing | PRKCE; CSNK1E; MAPK1; CAPNS1; AKT2; CAPN2; |
| | CAPN1; MAPK3; MAPK13; MAPT; MAPK14; AKT1; |
| | PSEN1; CSNK1A1; GSK3B; AKT3; APP |
| IL-4 Signaling | AKT2; PIK3CA; PIK3CB; PIK3C3; IRS1; KRAS; SOCS1; |
| | PTPN6; NR3C1; PIK3C2A; JAK1; AKT1; JAK2; PIK3R1; |
| | FRAP1; AKT3; RPS6KB1 |
| Cell Cycle: G2/M DNA | EP300; PCAF; BRCA1; GADD45A; PLK1; BTRC; |
| Damage Checkpoint | CHEK1; ATR; CHEK2; YWHAZ; TP53; CDKN1A; |
| Regulation | PRKDC; ATM; SFN; CDKN2A |
| Nitric Oxide Signaling in | KDR; FLT1; PGF; AKT2; PIK3CA; PIK3CB; PIK3C3; |
| the Cardiovascular System | CAV1; PRKCD; NOS3; PIK3C2A; AKT1; PIK3R1; |
| | VEGFA; AKT3; HSP90AA1 |
| Purine Metabolism | NME2; SMARCA4; MYH9; RRM2; ADAR; EIF2AK4; |
| | PKM2; ENTPD1; RAD51; RRM2B; TJP2; RAD51C; |
| | NT5E; POLD1; NME1 |
| cAMP-mediated Signaling | RAP1A; MAPK1; GNAS; CREB1; CAMK2A; MAPK3; |
| | SRC; RAF1; MAP2K2; STAT3; MAP2K1; BRAF; ATF4 |
| Mitochondrial Dysfunction | SOD2; MAPK8; CASP8; MAPK10; MAPK9; CASP9; |
| | PARK7; PSEN1; PARK2; APP; CASP3 |
| Notch Signaling | HES1; JAG1; NUMB; NOTCH4; ADAM17; NOTCH2; |
| | PSEN1; NOTCH3; NOTCH1; DLL4 |
| Endoplasmic Reticulum | HSPA5; MAPK8; XBP1; TRAF2; ATF6; CASP9; ATF4; |
| Stress Pathway | EIF2AK3; CASP3 |
| Pyrimidine Metabolism | NME2; AICDA; RRM2; EIF2AK4; ENTPD1; RRM2B; |
| | NT5E; POLD1; NME1 |
| Parkinson's Signaling | UCHL1; MAPK8; MAPK13; MAPK14; CASP9; PARK7; |
| | PARK2; CASP3 |
| Cardiac & Beta Adrenergic | GNAS; GNAQ; PPP2R1A; GNB2L1; PPP2CA; PPP1CC; |
| Signaling | PPP2R5C |
| Glycolysis/Gluconeogenesis | HK2; GCK; GPI; ALDH1A1; PKM2; LDHA; HK1 |
| Interferon Signaling | IRF1; SOCS1; JAK1; JAK2; IFITM1; STAT1; IFIT3 |
| Sonic Hedgehog Signaling | ARRB2; SMO; GLI2; DYRK1A; GLI1; GSK3B; DYRK1B |
| Glycerophospholipid | PLD1; GRN; GPAM; YWHAZ; SPHK1; SPHK2 |
| Metabolism | |
| Phospholipid Degradation | PRDX6; PLD1; GRN; YWHAZ; SPHK1; SPHK2 |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision Repair | ERCC5; ERCC4; XPA; XPC; ERCC1 |
| Pathway | |
| Starch and Sucrose | UCHL1; HK2; GCK; GPI; HK1 |
| Metabolism | |
| Aminosugars Metabolism | NQO1; HK2; GCK; HK1 |
| Arachidonic Acid | PRDX6; GRN; YWHAZ; CYP1B1 |
| Metabolism | |
| Circadian Rhythm Signaling | CSNK1E; CREB1; ATF4; NR1D1 |
| Coagulation System | BDKRB1; F2R; SERPINE1; F3 |
| Dopamine Receptor | PPP2R1A; PPP2CA; PPP1CC; PPP2R5C |
| Signaling | |
| Glutathione Metabolism | IDH2; GSTP1; ANPEP; IDH1 |
| Glycerolipid Metabolism | ALDH1A1; GPAM; SPHK1; SPHK2 |
| Linoleic Acid Metabolism | PRDX6; GRN; YWHAZ; CYP1B1 |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine and | PRDX6; PRDX1; TYR |
| Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of Xenobiotics | GSTP1; CYP1B1 |
| by Cytochrome p450 | |
| Methane Metabolism | PRDX6; PRDX1 |
| Phenylalanine Metabolism | PRDX6; PRDX1 |
| Propanoate Metabolism | ALDH1A1; LDHA |
| Selenoamino Acid | PRMT5; AHCY |
| Metabolism | |
| Sphingolipid Metabolism | SPHK1; SPHK2 |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated Oxidative | PRDX1 |
| Stress Response | |
| Pentose Phosphate | GPI |
| Pathway | |
| Pentose and Glucuronate | UCHL1 |
| Interconversions | |
| Retinol Metabolism | ALDH1A1 |
| Riboflavin Metabolism | TYR |
| Tyrosine Metabolism | PRMT5, TYR |
| Ubiquinone Biosynthesis | PRMT5 |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; cnr2; Grk2; |
| | Trpa1; Pomc; Cgrp; Crf; Pka; Era; Nr2b; TRPM5; Prkaca; |
| | Prkacb; Prkar1a; Prkar2a |
| Mitochondrial Function | AIF; CytC; SMAC (Diablo); Aifm-1; Aifm-2 |
| Developmental Neurology | BMP-4; Chordin (Chrd); Noggin (Nog); WNT (Wnt2; |
| | Wnt2b; Wnt3a; Wnt4; Wnt5a; Wnt6; Wnt7b; Wnt8b; |
| | Wnt9a; Wnt9b; Wnt10a; Wnt10b; Wnt16); beta-catenin; |
| | Dkk-1; Frizzled related proteins; Otx-2; Gbx2; FGF-8; |
| | Reelin; Dab1; unc-86 (Pou4fl or Brn3a); Numb; Reln |

Chimeric Antigen Receptors and Engineered T Cell Receptors

The payload protein(s) can comprise a chimeric antigen receptor (CAR) or T-cell receptor (TCR). In some embodiments, the CAR comprises a T-cell receptor (TCR) antigen binding domain. The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. The terms “CAR” and “CAR molecule” are used interchangeably. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some embodiments, the set of polypeptides are in the same polypeptide chain (e.g., comprise a chimeric fusion protein). In some aspects, the set of polypeptides are contiguous with each other. In some embodiments, the set of polypeptides are not contiguous with each other, e.g., are in different polypeptide chains. In some embodiments, the set of polypeptides include a dimerization switch that, upon the presence of a dimerization molecule, can couple the polypeptides to one another, e.g., can couple an antigen binding domain to an intracellular signaling domain. In one aspect, the stimulatory molecule is the zeta chain associated with the T cell receptor complex. In one aspect, the cytoplasmic signaling domain further comprises one or more functional signaling domains derived from at least one costimulatory molecule as defined below. In some embodiments, the costimulatory molecule is chosen from the costimulatory molecules described herein, e.g., 4-1BB (i.e., CD137), CD27 and/or CD28. In some embodiments, the CAR comprises a chimeric fusion protein comprising an extracellular antigen binding domain, a transmembrane domain and an intracellular signaling domain comprising a functional signaling domain derived from a stimulatory molecule. In some embodiments, the CAR comprises a chimeric fusion protein comprising an extracellular antigen binding domain, a transmembrane domain and an intracellular signaling domain comprising a functional signaling domain derived from a costimulatory molecule and a functional signaling domain derived from a stimulatory molecule. In some embodiments, the CAR comprises a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen binding domain, a transmembrane domain and an intracellular signaling domain comprising at least two functional signaling domains derived from one or more costimulatory molecule(s) and a functional signaling domain derived from a stimulatory molecule. In some embodiments the CAR comprises an optional leader sequence at the amino-terminus (N-ter) of the CAR fusion protein. In some embodiments, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

The CAR and/or TCR can comprise one or more of an antigen binding domain, a transmembrane domain, and an intracellular signaling domain. The CAR or TCR further can comprise a leader peptide. The TCR further can comprise a constant region and/or CDR4. The term “signaling domain” refers to the functional portion of a protein which acts by transmitting information within the cell to regulate cellular activity via defined signaling pathways by generating second messengers or functioning as effectors by responding to such messengers. An “intracellular signaling domain,” as the term is used herein, refers to an intracellular portion of a molecule. The intracellular signaling domain generates a signal that promotes an immune effector function of the CAR containing cell, e.g., a CART cell. Examples of immune effector function, e.g., in a CART cell, include cytolytic activity and helper activity, including the secretion of cytokines. In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule. A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, common FcR gamma (FCER1G), Fc gamma RIIa, FcR beta (Fc Epsilon R1b), CD3 gamma, CD3 delta, CD3 epsilon, CD79a, CD79b, DAP10, and DAP12.

The intracellular signaling domain can comprise a primary signaling domain, a costimulatory domain, or both of a primary signaling domain and a costimulatory domain. The cytoplasmic domain or region of the CAR includes an intracellular signaling domain. An intracellular signaling domain is generally responsible for activation of at least one of the normal effector functions of the immune cell in which the CAR has been introduced. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The term “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are contribute to an efficient immune response. Costimulatory molecules include, but are not limited to an MHC class I molecule, BTLA and a Toll ligand receptor, as well as OX40, CD27, CD28, CD5, ICAM-1, LFA-1 (CD11a/CD18), ICOS (CD278), and 4-1BB (CD137). Further examples of such costimulatory molecules include CD5, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83. A costimulatory intracellular signaling domain can be the intracellular portion of a costimulatory molecule. A costimulatory molecule can be represented in the following protein families: TNF receptor proteins, Immunoglobulin-like proteins, cytokine receptors, integrins, signaling lymphocytic activation molecules (SLAM proteins), and activating NK cell receptors. The intracellular signaling domain can comprise the entire intracellular portion, or the entire native intracellular signaling domain, of the molecule from which it is derived, or a functional fragment or derivative thereof.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability. It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain). A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs. The primary signaling domain can comprise a functional signaling domain of one or more proteins selected from the group consisting of CD3 zeta, CD3 gamma, CD3 delta, CD3 epsilon, common FcR gamma (FCER1G), FcR beta (Fc Epsilon R1b), CD79a, CD79b, Fcgamma RIIa, DAP10, and DAP12, or a functional variant thereof.

In some embodiments, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue. The costimulatory domain can comprise a functional domain of one or more proteins selected from the group consisting of CD27, CD28, 4-1BB (CD137), OX40, CD28-OX40, CD28-4-1BB, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D, or a functional variant thereof.

The portion of the CAR comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody, or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In some embodiments, the antigen binding domain of a CAR composition of the invention comprises an antibody fragment. In a further aspect, the CAR comprises an antibody fragment that comprises a scFv.

In some embodiments, the CAR of the invention comprises a target-specific binding element otherwise referred to as an antigen binding domain. The choice of moiety depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen binding domain in a CAR of the invention include those associated with viral, bacterial and parasitic infections, autoimmune disease and cancer cells.

In some embodiments, the CAR-mediated T-cell response can be directed to an antigen of interest by way of engineering an antigen binding domain that specifically binds a desired antigen into the CAR. In some embodiments, the portion of the CAR comprising the antigen binding domain comprises an antigen binding domain that targets a tumor antigen, e.g., a tumor antigen described herein. The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment. In some embodiments, the antigen binding domain comprises a humanized antibody or an antibody fragment. In some aspects, a non-human antibody is humanized, where specific sequences or regions of the antibody are modified to increase similarity to an antibody naturally produced in a human or fragment thereof. In some embodiments, the antigen binding domain is humanized.

The antigen binding domain can comprise an antibody, an antibody fragment, an scFv, a Fv, a Fab, a (Fab′)2, a single domain antibody (SDAB), a VH or VL domain, a camelid VHH domain, a Fab, a Fab′, a F(ab′)2, a Fv, a scFv, a dsFv, a diabody, a triabody, a tetrabody, a multispecific antibody formed from antibody fragments, a single-domain antibody (sdAb), a single chain comprising cantiomplementary scFvs (tandem scFvs) or bispecific tandem scFvs, an Fv construct, a disulfide-linked Fv, a dual variable domain immunoglobulin (DVD-Ig) binding protein or a nanobody, an aptamer, an affibody, an affilin, an affitin, an affimer, an alphabody, an anticalin, an avimer, a DARPin, a Fynomer, a Kunitz domain peptide, a monobody, or any combination thereof.

In some embodiments, the antigen binding domain is a T cell receptor (“TCR”), or a fragment thereof, for example, a single chain TCR (scTCR). Methods to make such TCRs are known in the art. See, e.g., Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Vα and Vβ genes from a T cell clone linked by a linker (e.g., a flexible peptide). This approach is very useful to cancer associated target that itself is intracelluar, however, a fragment of such antigen (peptide) is presented on the surface of the cancer cells by MHC.

In some embodiments, the antigen binding domain is a multispecific antibody molecule. In some embodiments, the multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope.

The antigen binding domain can be configured to bind to a tumor antigen. The terms “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell. In some embodiments, the CARs of the present invention includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+ T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The tumor antigen can be a solid tumor antigen. The tumor antigen can be selected from the group consisting of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDG1cp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis (Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDG1cp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; survivin; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

The tumor antigen can be selected from the group comprising CD150, 5T4, ActRIIA, B7, BMCA, CA-125, CCNA1, CD123, CD126, CD138, CD14, CD148, CD15, CD19, CD20, CD200, CD21, CD22, CD23, CD24, CD25, CD26, CD261, CD262, CD30, CD33, CD362, CD37, CD38, CD4, CD40, CD40L, CD44, CD46, CD5, CD52, CD53, CD54, CD56, CD66a-d, CD74, CD8, CD80, CD92, CE7, CS-1, CSPG4, ED-B fibronectin, EGFR, EGFRvIII, EGP-2, EGP-4, EPHa2, ErbB2, ErbB3, ErbB4, FBP, GD2, GD3, HER1-HER2 in combination, HER2-HER3 in combination, HERV-K, HIV-1 envelope glycoprotein gp120, HIV-1 envelope glycoprotein gp41, HLA-DR, HM1.24, HMW-MAA, Her2, Her2/neu, IGF-1R, IL-11 Ralpha, IL-13R-alpha2, IL-2, IL-22R-alpha, IL-6, IL-6R, Ia, Ii, L1-CAM, L1-cell adhesion molecule, Lewis Y, L1-CAM, MAGE A3, MAGE-A1, MART-1, MUC1, NKG2C ligands, NKG2D Ligands, NY-ESO-1, OEPHa2, PIGF, PSCA, PSMA, ROR1, T101, TAC, TAG72, TIM-3, TRAIL-R1, TRAIL-R1 (DR4), TRAIL-R2 (DR5), VEGF, VEGFR2, WT-1, a G-protein coupled receptor, alphafetoprotein (AFP), an angiogenesis factor, an exogenous cognate binding molecule (ExoCBM), oncogene product, anti-folate receptor, c-Met, carcinoembryonic antigen (CEA), cyclin (D1), ephrinB2, epithelial tumor antigen, estrogen receptor, fetal acethycholine e receptor, folate binding protein, gp100, hepatitis B surface antigen, kappa chain, kappa light chain, kdr, lambda chain, livin, melanoma-associated antigen, mesothelin, mouse double minute 2 homolog (MDM2), mucin 16 (MUC16), mutated p53, mutated ras, necrosis antigens, oncofetal antigen, ROR2, progesterone receptor, prostate specific antigen, tEGFR, tenascin, β2-Microglobulin, Fc Receptor-like 5 (FcRL5), or molecules expressed by HIV, HCV, HBV, or other pathogens.

The antigen binding domain can be connected to the transmembrane domain by a hinge region. In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge (e.g., an IgG4 hinge, an IgD hinge), a GS linker (e.g., a GS linker described herein), a KIR2DS2 hinge or a CD8a hinge.

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In some embodiments, the transmembrane domain is one that is associated with one of the other domains of the CAR e.g., in one embodiment, the transmembrane domain may be from the same protein that the signaling domain, costimulatory domain or the hinge domain is derived from. In some embodiments, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins, e.g., to minimize interactions with other members of the receptor complex. In some embodiments, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CAR-expressing cell.

The transmembrane domain can comprise a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7Ra, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and NKG2C, or a functional variant thereof. The transmembrane domain may be derived either from a natural or from a recombinant source. Where the source is natural, the domain may be derived from any membrane-bound or transmembrane protein. In some embodiments the transmembrane domain is capable of signaling to the intracellular domain(s) whenever the CAR has bound to a target.

### Pharmaceutically Acceptable Compositions and Methods

Disclosed herein include pharmaceutical compositions. In some embodiments, the pharmaceutical composition comprises: a composition provided herein (e.g., a nucleic acid composition, a population of sender cells), wherein the pharmaceutical composition further comprises one or more pharmaceutically acceptable carriers, diluents and/or excipients.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth: (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (1) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Formulations useful in the methods of the present invention include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient (e.g., sender cells, nucleic acid composition) which can be combined with a carrier material to produce a single dosage form will vary depending upon the host being treated, the particular mode of administration. The amount of active ingredient, which can be combined with a carrier material to produce a single dosage form will generally be that amount of the nucleic acid composition and/or sender cells which produces a therapeutic effect. Generally, out of one hundred percent, this amount will range from about 1% to about 99% of active ingredient, preferably from about 5% to about 70%, most preferably from about 10% to about 30%.

Disclosed herein include methods of treating or preventing a disease or disorder in a subject in need thereof. In some embodiments, the method comprises: administering to the subject an effective amount of a nucleic acid composition disclosed herein, a pharmaceutical composition disclosed herein, or the sender cells disclosed herein, thereby treating or preventing the disease or disorder in the subject. In some embodiments, administering comprises: (i) isolating one or more cells from the subject; (ii) contacting (e.g,. transfecting) said one or more cells with a nucleic acid composition disclosed herein, thereby generating sender cells; and (iii) administering the one or more sender cells into a subject after the contacting step. The method can comprise: administering to the subject an effective amount of a transactivator, a bridge protein, a pro-death agent, or any combination thereof. The sender sends can be configured to travel to and/or accumulate at a target site of a subject. In some embodiments, nucleic acid composition(s) are administered to a subject to generate sender cells in vivo. Alternatively, in some embodiments, sender cells are generated (e.g., by incorporating the nucleic acid composition(s) provided herein) outside the body of the subject and are subsequently administrated to the subject.

The disclosed sender cells described herein may be included in a composition for therapy. In some embodiments, the composition comprises a population of disclosed sender cells. The composition may include a pharmaceutical composition and further include a pharmaceutically acceptable carrier. A therapeutically effective amount of the pharmaceutical composition comprising the disclosed sender cells may be administered. The cells provided herein may be administered either alone, or as a pharmaceutical composition in combination with diluents and/or with other components such as IL-2 or other cytokines or cell populations. Ex vivo procedures are well known in the art. Briefly, cells are isolated from a mammal (e.g., a human) and genetically modified (i.e., transduced or transfected in vitro) with a nucleic acid composition (e.g., a vector) disclosed herein or a composition disclosed herein, thereby generating an engineered population of cells. The disclosed sender cells can be administered to a mammalian recipient to provide a therapeutic benefit. The mammalian recipient may be a human and the disclosed sender cells can be autologous with respect to the recipient. Alternatively, the disclosed sender cells can be allogeneic, syngeneic or xenogeneic with respect to the recipient.

Administering can comprise aerosol delivery, nasal delivery, vaginal delivery, rectal delivery, buccal delivery, ocular delivery, local delivery, topical delivery, intracisternal delivery, intraperitoneal delivery, oral delivery, intramuscular injection, intravenous injection, subcutaneous injection, intranodal injection, intratumoral injection, intraperitoneal injection, intradermal injection, or any combination thereof. The disclosed sender cells can be administered at a therapeutically effective amount. For example, a therapeutically effective amount of the disclosed sender cells can be at least about 10² cells, at least about 10³ cells, at least about 10⁴ cells, at least about 10⁵ cells, at least about 10⁶ cells, at least about 10⁷ cells, at least about 10⁸ cells, at least about 10⁹, or at least about 10¹⁰. In another embodiment, the therapeutically effective amount of the disclosed sender cells is about 10⁴ cells, about 10⁵ cells, about 10⁶ cells, about 10⁷ cells, or about 10⁸ cells. In one particular embodiment, the therapeutically effective amount of the disclosed sender cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg.

Actual dosage levels of the active ingredients in the pharmaceutical compositions of this invention may be determined by the methods of the present invention so as to obtain an amount of the active ingredient, which is effective to achieve the desired therapeutic response for a particular subject, composition, and mode of administration, without being toxic to the subject.

Also provided herein are kits comprising one or more compositions described herein, in suitable packaging, and may further comprise written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. A kit may comprise one or more unit doses described herein.

The disease or disorder can be a blood disease, an immune disease, a neurological disease or disorder, a cancer, an infectious disease, a genetic disease, a disorder caused by aberrant mtDNA, a metabolic disease, a disorder caused by aberrant cell cycle, a disorder caused by aberrant angiogenesis, a disorder cause by aberrant DNA damage repair, a solid tumor, or any combination thereof. The disease or disorder can be an infectious disease selected from the group consisting of an Acute Flaccid Myelitis (AFM), Anaplasmosis, Anthrax, Babesiosis, Botulism, Brucellosis, Campylobacteriosis, Carbapenem-resistant Infection, Chancroid, Chikungunya Virus Infection, Chlamydia, Ciguatera, Difficile Infection, Perfringens, Coccidioidomycosis fungal infection, coronavirus infection, Covid-19 (SARS-CoV-2), Creutzfeldt-Jacob Disease/transmissible spongiform encephalopathy, Cryptosporidiosis (Crypto), Cyclosporiasis, Dengue 1,2,3 or 4, Diphtheria, E. coli infection/Shiga toxin-producing (STEC), Eastern Equine Encephalitis, Hemorrhagic Fever (Ebola), Ehrlichiosis, Encephalitis, Arboviral or parainfectious, Non-Polio Enterovirus, D68 Enteroviru (EV-D68), Giardiasis, Glanders, Gonococcal Infection, Granuloma inguinale, Haemophilus influenza disease Type B (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster (Shingles), Histoplasmosis infection, Human Immunodeficiency Virus/AIDS (HIV/AIDS), Human Papillomavirus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LGV), Malaria, Measles, Meliodosis, Meningitis (Viral), Meningococcal Disease (Meningitis (Bacterial)), Middle East Respiratory Syndrome Coronavirus (MERS-CoV), Mumps, Norovirus, Pediculosis, Pelvic Inflammatory Disease (PID), Pertussis (Whooping Cough), Plague (Bubonic, Septicemic, Pneumonic), Pneumococcal Disease (Pneumonia), Poliomyelitis (Polio), Powassan, Psittacosis, Pthiriasis, Pustular Rash diseases (Small pox, monkeypox, cowpox), Q-Fever, Rabies, Rickettsiosis (Rocky Mountain Spotted Fever), Rubella (German Measles), Salmonellosis gastroenteritis (Salmonella), Scabies, Scombroid, Sepsis, Severe Acute Respiratory Syndrome (SARS), Shigellosis gastroenteritis (Shigella), Smallpox, Staphylococcal Infection Methicillin-resistant (MRSA), Staphylococcal Food Poisoning Enterotoxin B Poisoning (Staph Food Poisoning), Staphylococcal Infection Vancomycin Intermediate (VISA), Staphylococcal Infection Vancomycin Resistant (VRSA), Streptococcal Disease Group A (invasive) (Strep A (invasive), Streptococcal Disease, Group B (Strep-B), Streptococcal Toxic-Shock Syndrome STSS Toxic Shock, Syphilis (primary, secondary, early latent, late latent, congenital), Tetanus Infection, Trichomoniasis, Trichinosis Infection, Tuberculosis (TB), Tuberculosis Latent (LTBI), Tularemia, Typhoid Fever Group D, Vaginosis, Varicella (Chickenpox), Vibrio cholerae (Cholera), Vibriosis (Vibrio), Ebola Virus Hemorrhagic Fever, Lasa Virus Hemorrhagic Fever, Marburg Virus Hemorrhagic Fever, West Nile Virus, Yellow Fever, Yersenia, and Zika Virus Infection.

The disease can be associated with expression of a tumor-associated antigen. The disease associated with expression of a tumor antigen-associated can be selected from the group consisting of a proliferative disease, a precancerous condition, a cancer, and a non-cancer related indication associated with expression of the tumor antigen. The cancer can be selected from the group consisting of colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. The cancer can be a hematologic cancer chosen from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, or pre-leukemia.

Target Sites

In some embodiments, a target site of a subject comprises a site of disease or disorder or is proximate to a site of a disease or disorder. The target site can comprise a solid tumor. The location of the one or more sites of a disease or disorder can be predetermined, can be determined during the method, or both. The target site can be an immunosuppressive environment. The target site can comprise a tissue. The tissue can be inflamed tissue and/or infected tissue. The tissue can comprise adrenal gland tissue, appendix tissue, bladder tissue, bone, bowel tissue, brain tissue, breast tissue, bronchi, coronal tissue, ear tissue, esophagus tissue, eye tissue, gall bladder tissue, genital tissue, heart tissue, hypothalamus tissue, kidney tissue, large intestine tissue, intestinal tissue, larynx tissue, liver tissue, lung tissue, lymph nodes, mouth tissue, nose tissue, pancreatic tissue, parathyroid gland tissue, pituitary gland tissue, prostate tissue, rectal tissue, salivary gland tissue, skeletal muscle tissue, skin tissue, small intestine tissue, spinal cord, spleen tissue, stomach tissue, thymus gland tissue, trachea tissue, thyroid tissue, ureter tissue, urethra tissue, soft and connective tissue, peritoneal tissue, blood vessel tissue and/or fat tissue. The tissue can comprise: (i) grade I, grade II, grade III or grade IV cancerous tissue; (ii) metastatic cancerous tissue; (iii) mixed grade cancerous tissue; (iv) a sub-grade cancerous tissue; (v) healthy or normal tissue; and/or (vi) cancerous or abnormal tissue.

The method can comprise: applying a stimulus to a target site of the subject configured to induce RNA exporter expression, fusogen expression, and/or LN secretion at said target site (e.g., applying thermal energy to a target site of the subject sufficient to increase the local temperature of the target site). Applying thermal energy to a target site of the subject can comprise the application of one or more of focused ultrasound (FUS), magnetic hyperthermia, microwaves, infrared irradiation, liquid-based heating (e.g., intraperitoneal chemotherapy (HIPEC)), and contact heating.

Target sites can comprise target cells. In some embodiments, the receiver cells are target cells (e.g., cells of a unique cell type and/or unique cell state). The sender cells can be configured (e.g., via sender circuits) to release LNs in the vicinity of said target cells. Receiver circuits can be configured such that the therapeutic program is only activated in target cells (e.g., cells of a unique cell type and/or unique cell state).

At least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the plurality of LNs and/or payload molecules can be released at the target site. Less than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the plurality of LNs and/or payload molecules can be released at a location other than the target site. The ratio of the concentration of sender cells, receiver cells, LNs, and/or payload molecules at the subject's target site to the concentration of sender cells, receiver cells, LNs, and/or payload molecules in subject's blood, serum, or plasma can be about 2:1 to about 3000:1, about 2:1 to about 2000:1, about 2:1 to about 1000:1, or about 2:1 to about 600:1.

Additional Agents

In some embodiments, the method comprises administering one or more additional agents to the subject. In some embodiments, the one or more additional agents increases the efficacy of the population of sender cells and/or receiver cells. The one or more additional agents can comprise a protein phosphatase inhibitor, a kinase inhibitor, a cytokine, an inhibitor of an immune inhibitory molecule, and/or or an agent that decreases the level or activity of a TREG cell. The one or more additional agents can comprise an immune modulator, an anti-metastatic, a chemotherapeutic, a hormone or a growth factor antagonist, an alkylating agent, a TLR agonist, a cytokine antagonist, a cytokine antagonist, or any combination thereof. The one or more additional agents can comprise an agonistic or antagonistic antibody specific to a checkpoint inhibitor or checkpoint stimulator molecule such as PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, TIM-3.

### EXAMPLES

Some aspects of the embodiments discussed above are disclosed in further detail in the following examples, which are not in any way intended to limit the scope of the present disclosure.

### Example 1

### Cell-to-Cell Delivery of RNA Circuits

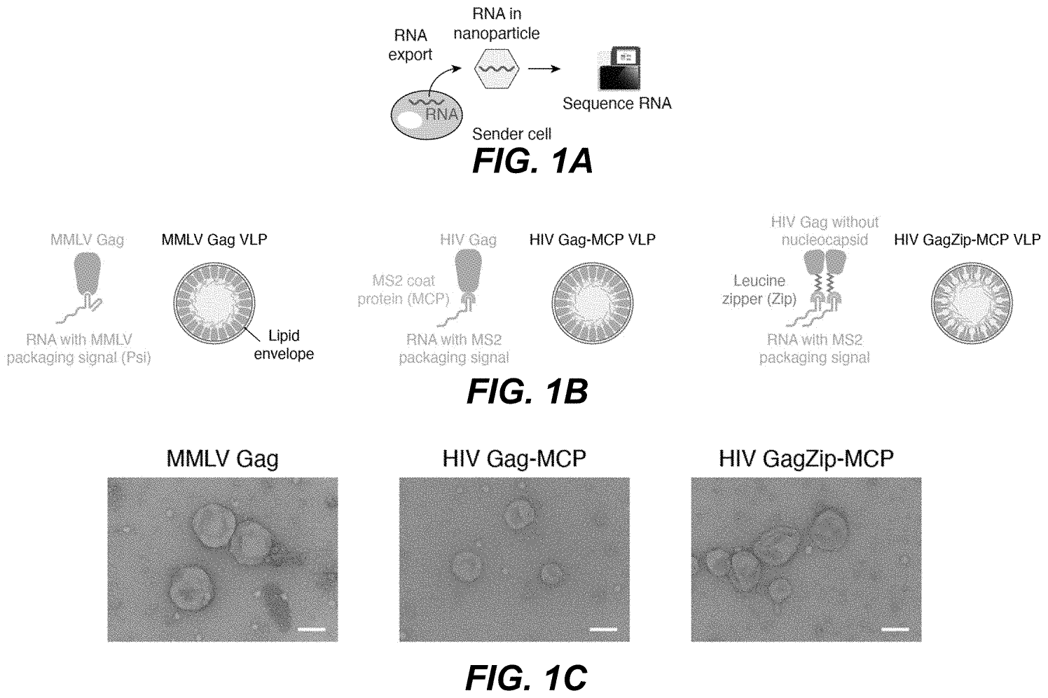

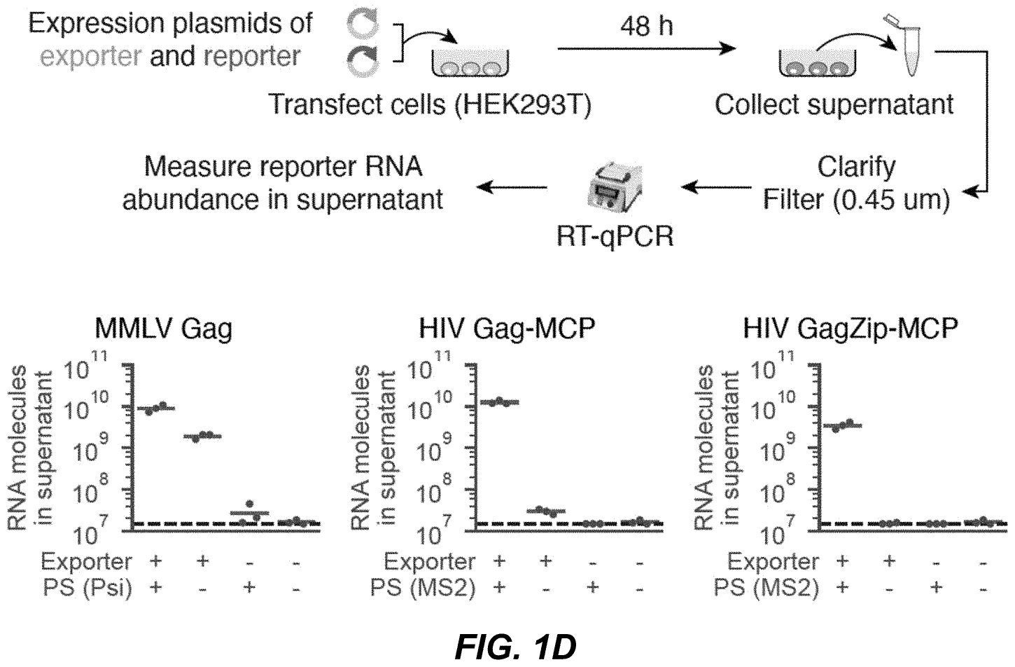

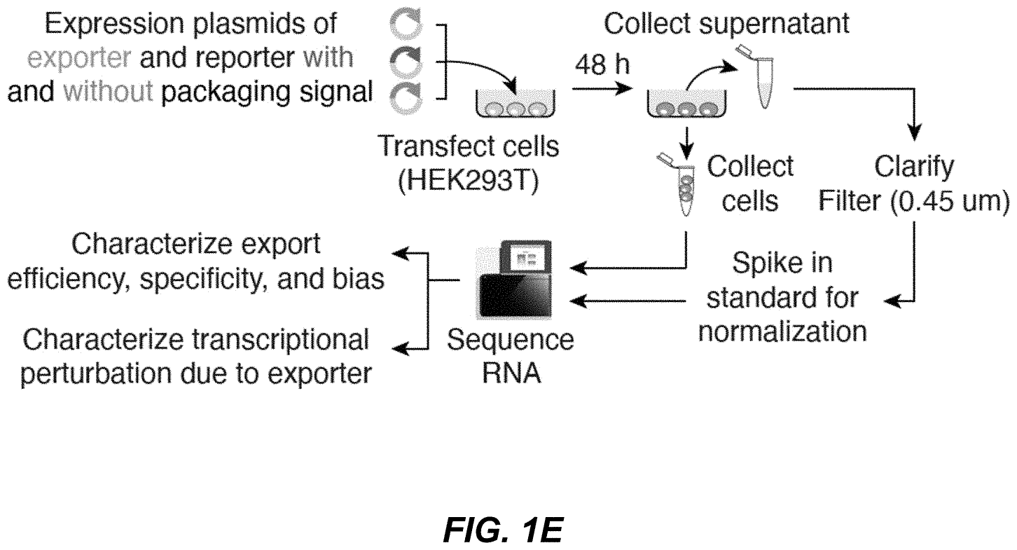

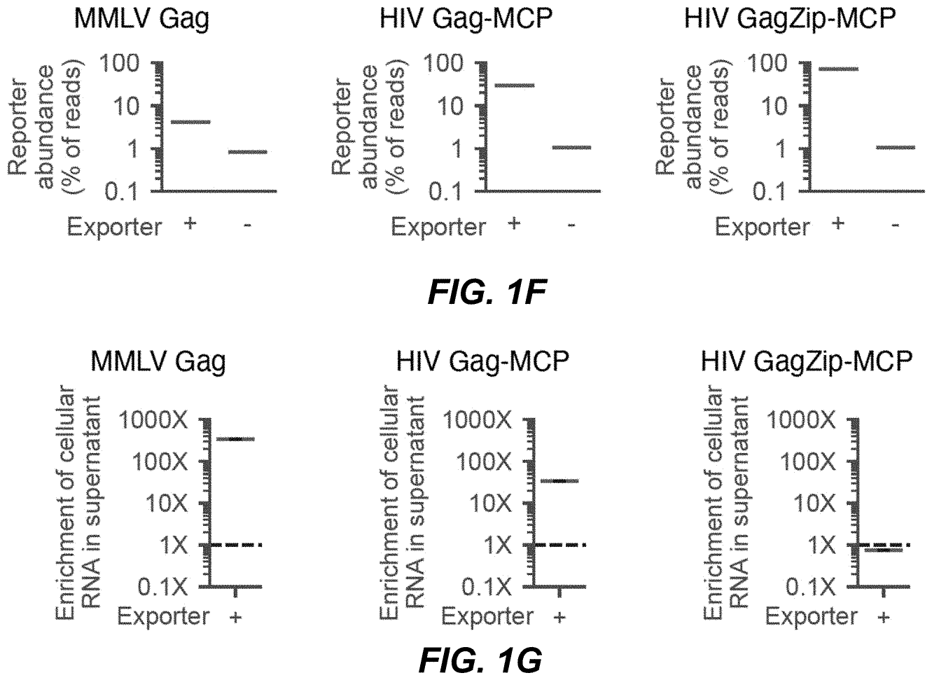

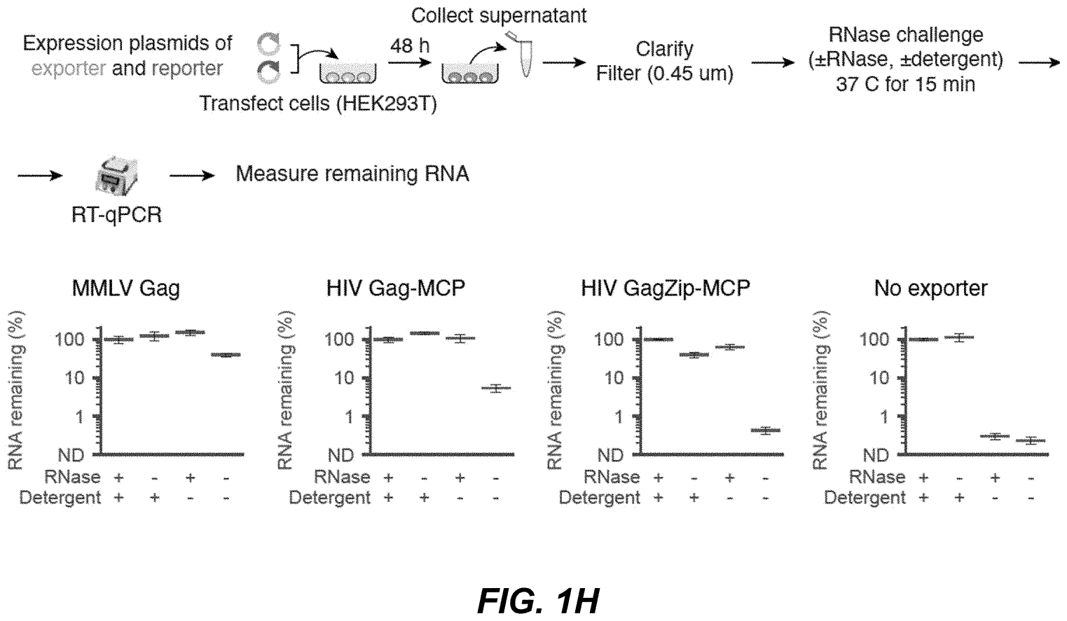

This example provides proof of concept for the compositions, methods, and systems provided herein. For proof of principle of RNA export in LNs by the disclosed RNA exporter proteins, reporter RNA molecules comprising packing signal(s) were employed. FIGS. 1A-1H depict non-limiting exemplary schematics and data related to engineered viral RNA exporters efficiently and specifically packaging, secreting, and protecting RNA. FIG. 1A depicts a non-limiting exemplary schematic of RNA export via secretion of LNs. RNA sequence encodes information about the sender cell which is read out by collecting and sequencing the exported reporter RNA molecule. FIG. 1B depicts non-limiting exemplary schematics of viral RNA exporter designs. Left, exporter based on Moloney Murine Leukemia Virus (MMLV) Gag capsid protein. MMLV Gag self-assembles to form viral capsid and packages RNA tagged with MMLV packaging signal (Psi) via interaction between MMLV Gag and Psi. Capsid is secreted from cell within a lipid envelope. Middle, exporter based on Human Immunodeficiency Virus (HIV) Gag capsid protein fused with RNA binding domain MS2 coat protein (MCP). HIV Gag domain self-assembles to form viral capsid. MCP binds RNA tagged with MS2 packaging signal, enabling specific packaging of target RNA. Right, exporter based on HIV Gag without nucleocapsid domain fused with leucine zipper dimerization domain from GCN4 (Zip), designated GagZip, which nucleates self-assembly via homodimerization of Zip to form viral capsid. RNA is specifically packaged via interaction of MS2 packaging signal with MCP. FIG. 1C depicts data related to negative stain transmission electron microscopy showing secretion of enveloped particles from HEK293T cells transfected with plasmids encoding RNA exporters. Scale bar, 100 nm. FIG. 1D depicts data related to the efficiency and specificity of RNA export to supernatant determined by reverse transcription followed by quantitative PCR (RT-qPCR). Top, schematic of experiment. HEK293T cells were transfected with expression plasmids of RNA exporter and reporter, the supernatant was collected after 48 hours, the supernatant was clarified by centrifugation at 3000 g for 5 minutes, the supernatant was passed through a 0.45 um filter, the RNA was extracted, and RT-qPCR was carried out to measure reporter RNA abundance in supernatant. Bottom, reporter RNA molecules detected in supernatant. Results indicate that viral RNA export systems efficiently release RNA from cells, and the specificity of release is improved by engineering specific RNA binding interactions into HIV Gag-MCP and GagZip-MCP. FIG. 1E depicts a non-limiting exemplary schematic of an experiment to characterize RNA exporters by sequencing. HEK293T cells were transfected with expression plasmids of RNA exporter and reporters with and without packaging signals, supernatant and cells were collected after 48 hours, the supernatant was clarified by centrifugation at 3000 g for 5 minutes, and the supernatant was passed through a 0.45 um filter. Then RNA standards were spiked into supernatant to enable determination of relative abundance of exported RNA across samples. RNA was extracted, libraries were prepared, and the RNA was sequenced. The reads from supernatant and cells were used to characterize export efficiency, specificity, and bias. The reads from cells were used to characterize transcriptional perturbations due to exporter expression. FIG. 1F depicts data related to the specificity of RNA export determined by sequencing. Improved packaging specificity of Gag-MCP and, to an even greater degree, GagZip-MCP results in a greater abundance of target reporter RNA among supernatant RNA, in comparison with MMLV Gag. This improvement may enhance the sensitivity of RNA export-based measurement systems. FIG. 1G depicts data related to the quantification of endogenous cellular RNA among supernatant RNA. Improved packaging specificity of Gag-MCP and, to an even greater degree, GagZip-MCP reduces the abundance of endogenous cellular RNA among supernatant RNA. FIG. 1H depicts data related to RNA exporters packaging and protecting RNA from degradation. Top, schematic of experiment to measure protection from RNase challenge. Bottom, RNA remaining after RNase challenge. Results show that RNA packaged and secreted by RNA exporters is protected from degradation, except in the presence of detergent, supporting a model of RNA export within lipid enveloped nanoparticles.

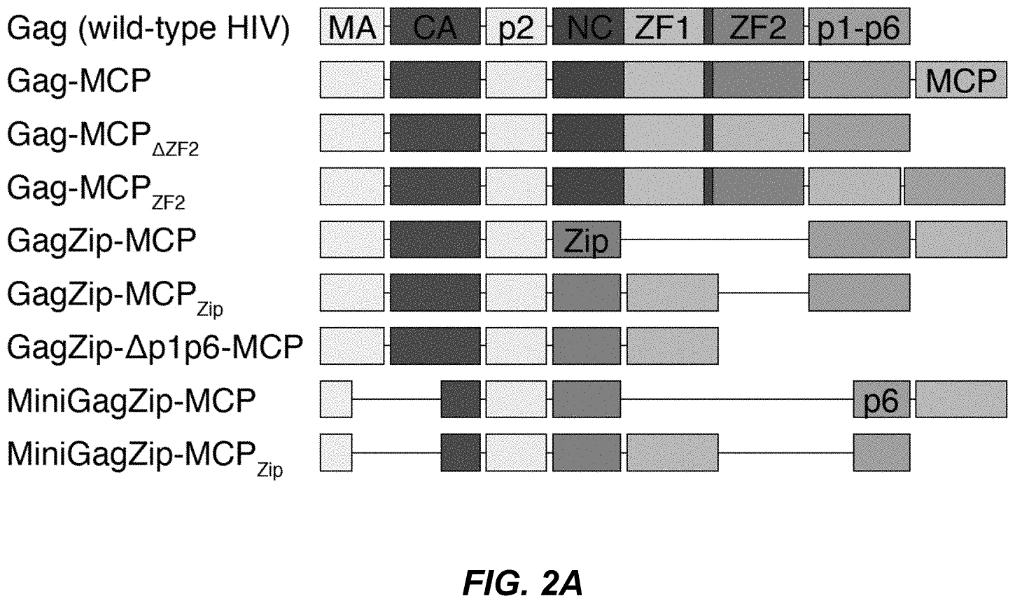

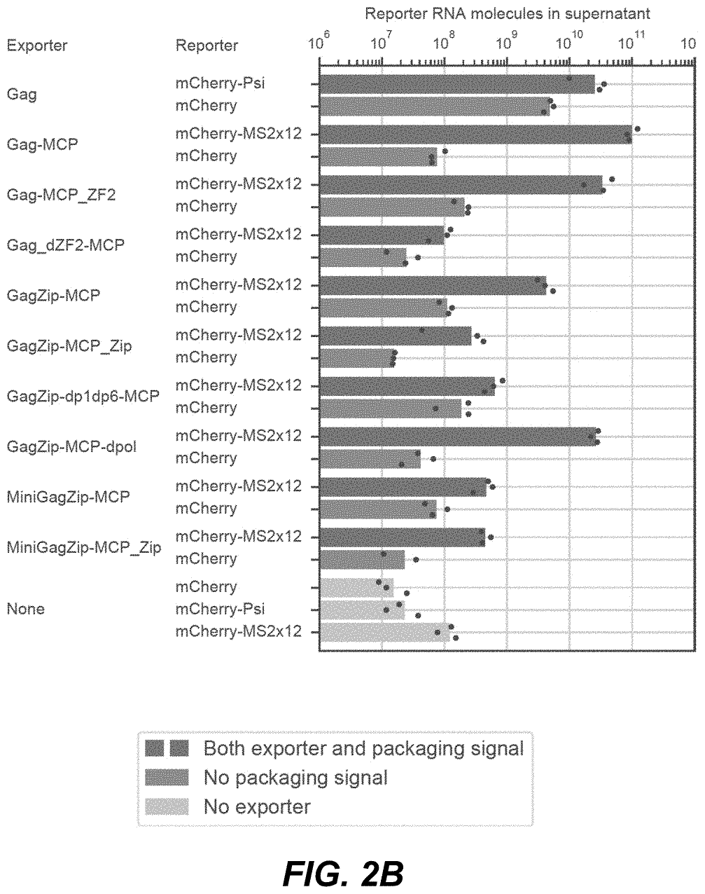

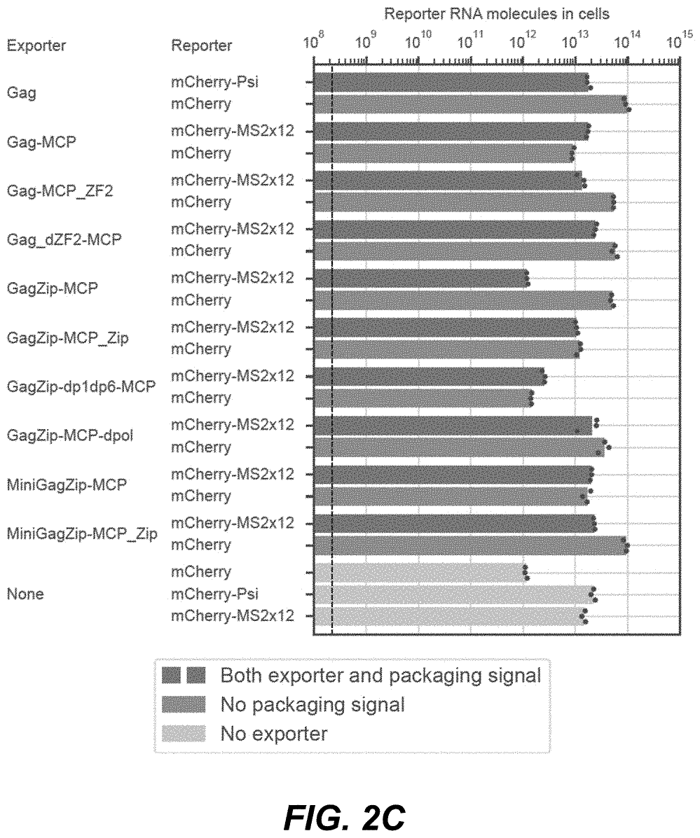

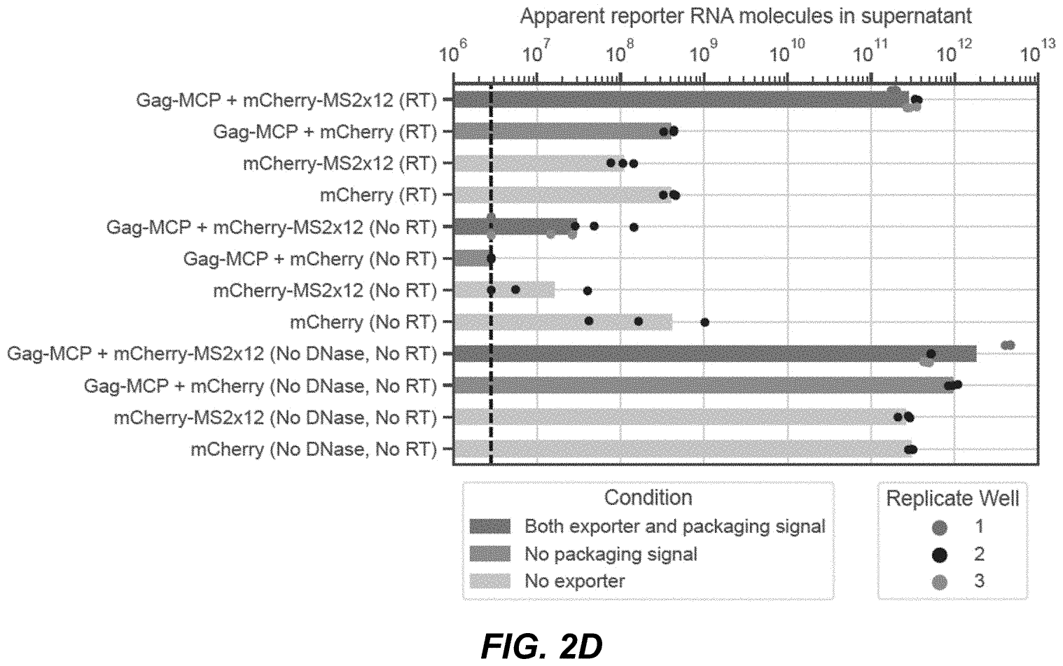

FIGS. 2A-2D depict non-limiting exemplary schematics and data related to the design and characterization of engineered viral RNA exporters. FIG. 2A depicts non-limiting exemplary domain architectures of viral RNA exporters. Left to right, N- to C-terminus. Colors indicate distinct protein domains. FIG. 2B depicts data related to the quantification of RNA export by viral RNA exporters with various architectures using reverse transcription followed by quantitative PCR (RT-qPCR). Cells were transfected with the indicated components of the RNA export system, supernatant was collected after 48 hours, RNA was extracted, and RT-qPCR was performed to measure the number of reporter RNA (mCherry mRNA) molecules in supernatant. Bars indicate mean of three technical replicates of qPCR, which are shown individually as dots. Colors indicate experimental condition, with grays showing key technical controls of omitting exporter or packaging signal. Results indicate that viral RNA export systems with a variety of protein architectures are capable of efficient and specific export of target RNA. Export can yield >1000-fold increased abundance of reporter RNA in supernatant, in comparison with no exporter and no packaging signal controls. FIG. 2C depicts data related to the quantification of RNA export system components in cellular RNA by RT-qPCR, using the same specimens as (B). Results indicate that expression of system components cannot explain the observed variation in RNA export efficiency in key technical controls, supporting that RNA export is exporter- and packaging signal-dependent. Dashed line indicates limit of detection, as determined by signal in control sample lacking DNA in transfection. FIG. 2D depicts data related to technical characteristics of RT-qPCR assay for RNA export to supernatant, indicating that assay faithfully and reproducibly measures RNA, not DNA, abundance. qPCR was performed with omission of key protocol steps: DNase treatment (labeled No DNase) or reverse transcription (labeled No RT). These conditions reveal that DNase treatment substantially reduces DNA contamination (which likely originates from transfected plasmid) to levels below the abundance of cDNA in samples lacking exporter. Thus, the assay faithfully measures RNA abundance within the relevant range for characterization of exporter performance. Assay was performed on three replicate wells, which were transfected independently and are indicated by different color dots, with three technical replicates of qPCR per well. Bars indicate mean of all replicates per condition. Consistency across replicate wells demonstrates that the assay is reproducible. Dashed line indicates limit of detection, as determined by signal in control sample lacking DNA in transfection.

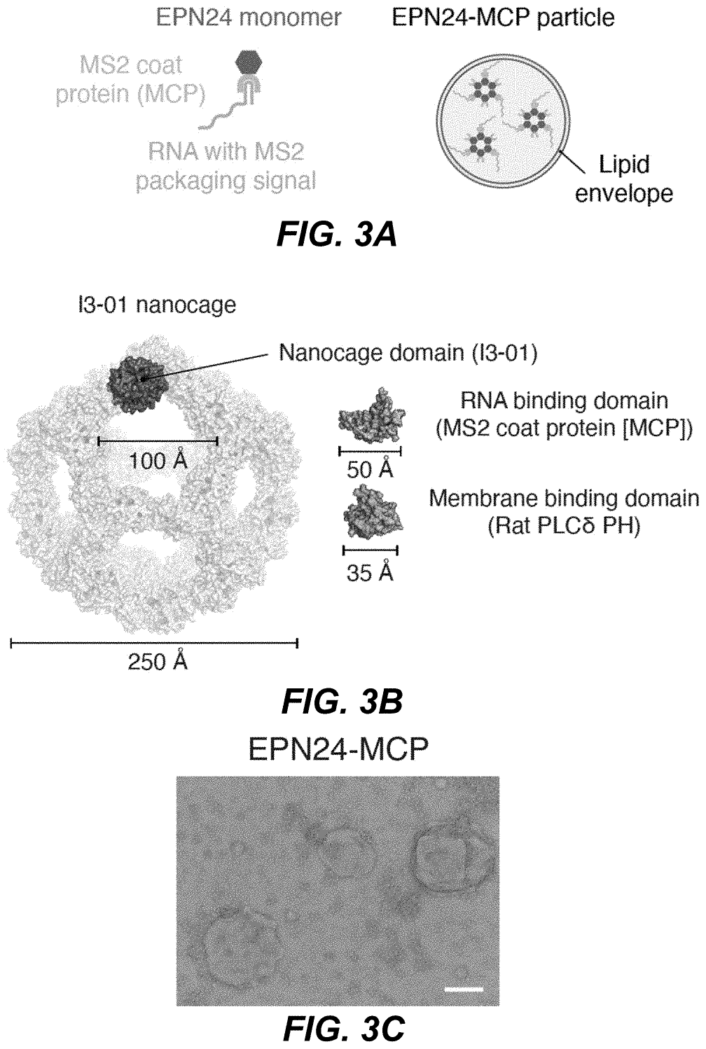

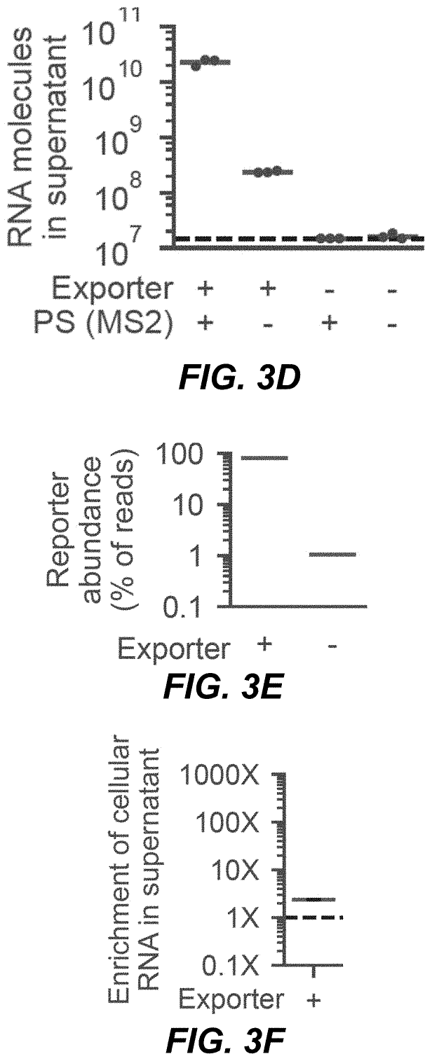

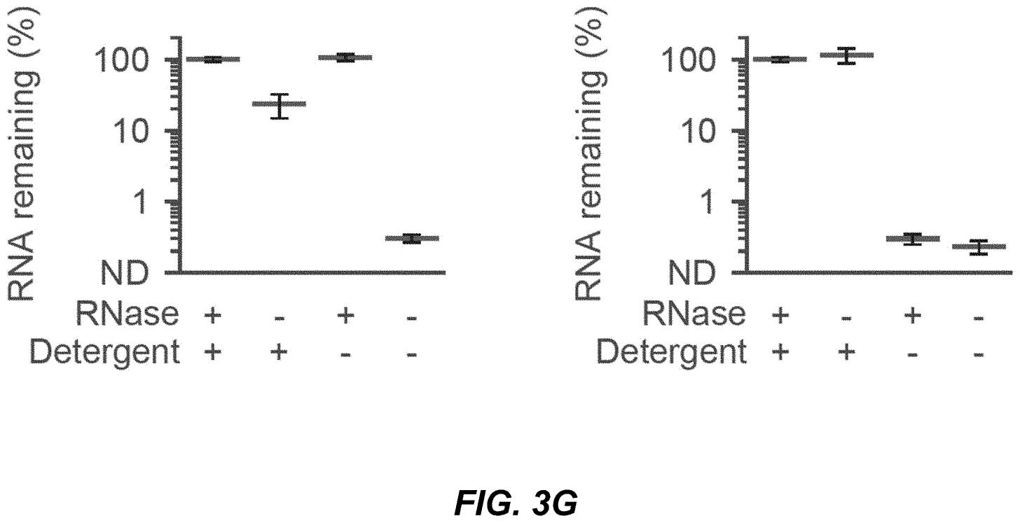

FIGS. 3A-3G depict non-limiting exemplary schematics and data related to synthetic non-viral RNA exporters efficiently and specifically packaging, secreting, and protecting RNA. FIG. 3A depicts a non-limiting exemplary schematic of synthetic non-viral RNA exporter design. Exporter comprises enveloped protein nanocage domain EPN24 fused with RNA binding domain MS2 coat protein (MCP). Exporter self-assembles to form nanocages, which package RNA tagged with MS2 packaging signal via interaction between MCP and MS2 aptamer. Exporter particles are composed of multiple nanocages secreted from the cell within an enveloped vesicle. FIG. 3B depicts a non-limiting exemplary protein design model of an RNA-exporting nanocage. Left, experimentally determined model of protein nanocage (PDB 5KP9). Monomer of I3-01 interaction domain is shown in purple. Right, x-ray crystal structures of MCP (PDB 1 MSC) and membrane binding domain (rat phospholipase C delta Pleckstrin Homology domain, PLCD PH; PDB 1 MAI), which are fused to I3-01 to form EPN24-MCP. Physical dimensions suggest that sufficient space exists within the I3-01 nanocage to accommodate the MCP and PLCD PH domains. FIG. 3C depicts data related to negative stain transmission electron microscopy showing secretion of enveloped particles from HEK293T cells transfected with plasmids encoding RNA exporter. Scale bar, 100 nm. FIG. 3D depicts data related to the efficiency and specificity of RNA export to supernatant determined by reverse transcription followed by quantitative PCR (RT-qPCR). Schematic of experiment is shown in FIG. 1D. Results indicate that synthetic RNA exporter EPN24-MCP releases reporter RNA from cells similarly efficiently and specifically as viral RNA exporters. FIG. 3E depicts data related to the specificity of RNA export determined by sequencing. Results show that the reporter RNA is the vast majority of exported RNA, indicating that RNA export is efficient and specific, similar to engineered viral RNA exporters. FIG. 3F depicts data related to the quantification of endogenous cellular RNA among supernatant RNA showing specific RNA export, similar to engineered viral RNA exporters. FIG. 3G depicts data related to the RNA exporters packaging and protecting RNA from degradation. Results show that RNA packaged and secreted by synthetic RNA exporters is protected from degradation, except in the presence of detergent, supporting a model of RNA export within lipid enveloped nanoparticles.

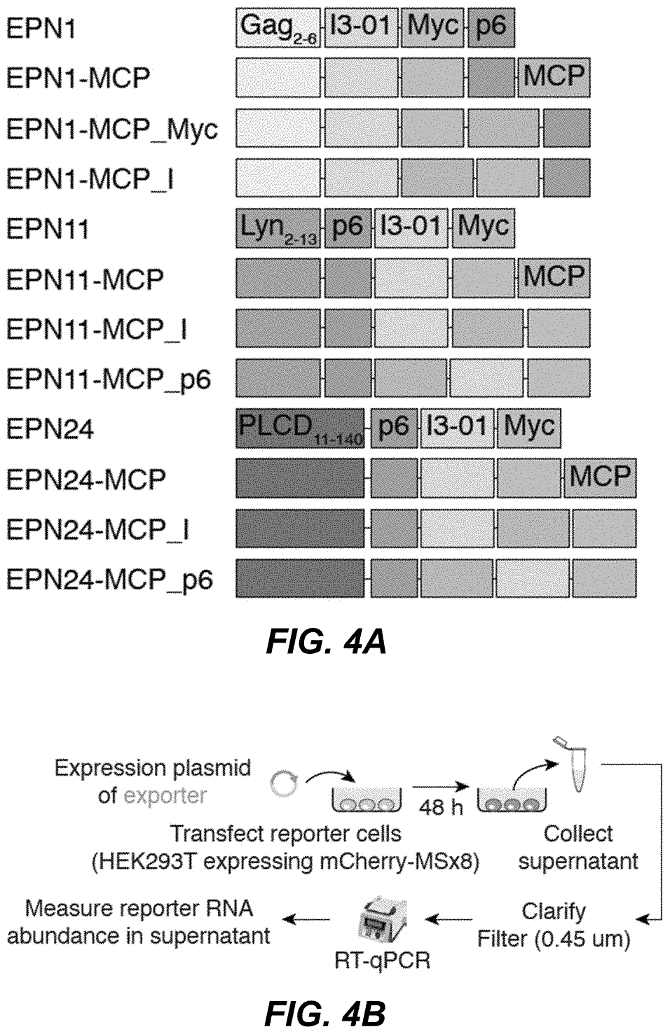

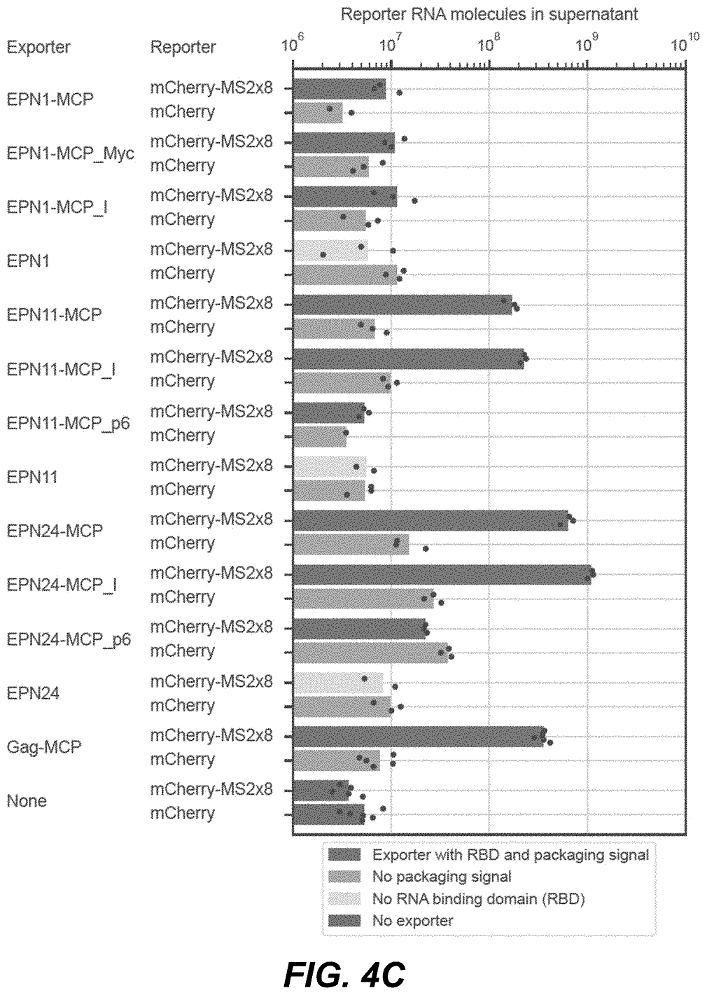

FIGS. 4A-4C depict non-limiting exemplary schematics and data related to the design and characterization of synthetic RNA exporters. FIG. 4A depicts non-limiting exemplary domain architectures of synthetic RNA exporters. Left to right, N- to C-terminus. Colors indicate distinct protein domains. Gag₂₋₆ is the myristoylation motif from HIV-1 NL4-3 Gag (amino acid (AA) residues 2-6). Lyn₂₋₁₃ is the myristoylation/palmitoylation motif from Lyn kinase (AA residues 2-13). PLCD₁₁₋₁₄₀ is the Pleckstrin Homology domain from rat phospholipase Cδ. p6 is the p6 domain from HIV-1 NL4-3 Gag. MCP is the MS2 coat protein, which serves as an RNA binding domain. MCP was fused in various positions within the RNA exporter sequence. FIG. 4B depicts a non-limiting exemplary schematic of assay for RNA export using stable reporter cell line. Lentivirus was used to create stable reporter cell lines that constitutively express mCherry either with or without the MS2 packaging signal, consisting of a tandem array of eight MS2 aptamers in the 3′ untranslated region (3′ UTR) of the mCherry transcript. To perform the assay, an expression plasmid of the RNA exporter was transfected into reporter cells, supernatant was collected after 48 hours, the supernatant was clarified by centrifugation at 3000 g for 5 minutes, the supernatant was passed through a 0.45 um filter, RNA was extracted, and RT-qPCR was carried out to measure reporter RNA abundance in supernatant. FIG. 4C depicts data related to the quantification of RNA export by synthetic RNA exporters with various architectures using RT-qPCR. Bars indicate mean of three technical replicates of qPCR, which are shown individually as dots. Colors indicate experimental condition, with grays showing key technical controls of omitting exporter, RNA binding domain, or packaging signal. Results indicate that synthetic RNA exporters with various architectures are capable of efficient and specific export of RNA. Export yields similarly increased abundance of reporter RNA in supernatant compared with the viral exporter Gag-MCP, suggesting similar export efficiency.

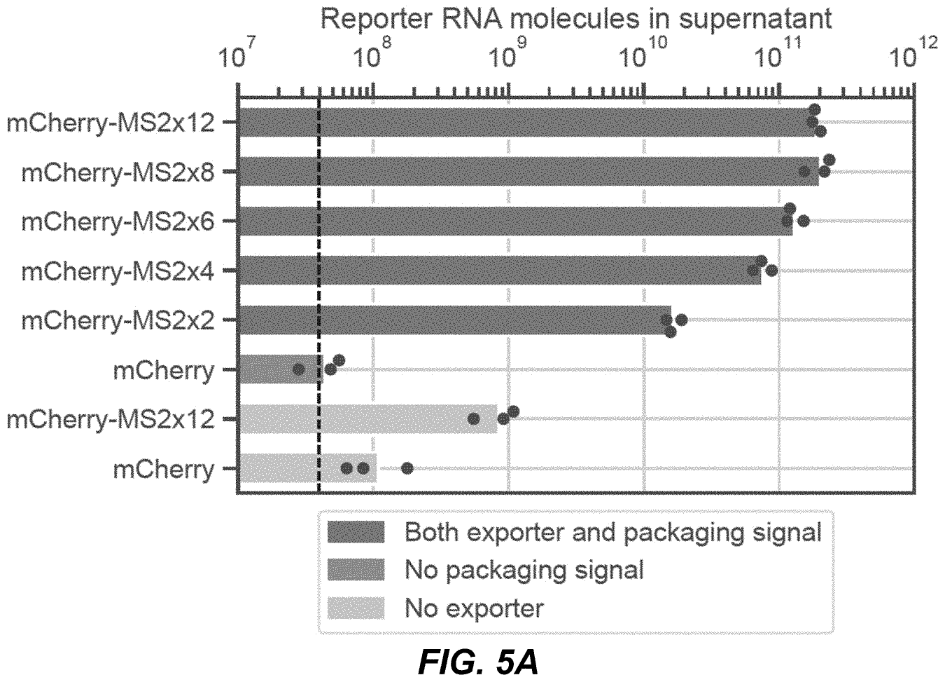

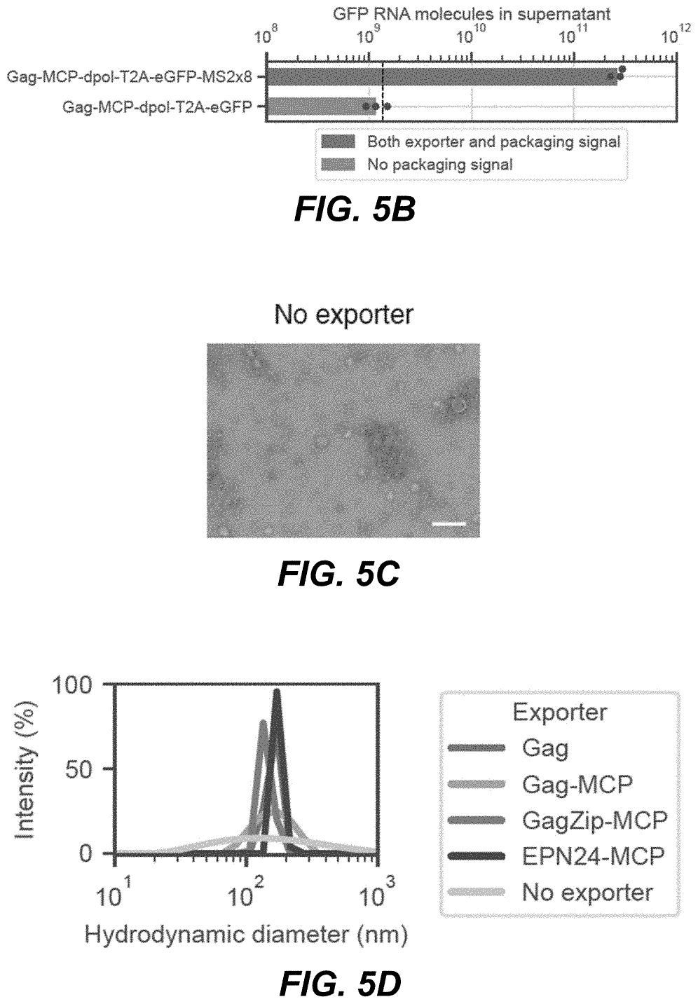

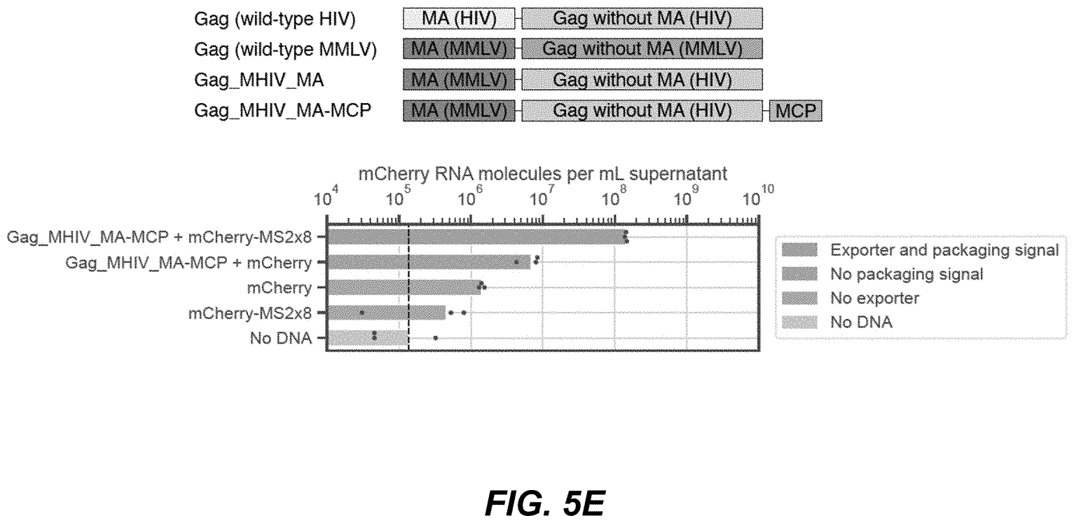

FIGS. 5A-5E depict data related to physical and functional features of RNA export systems. FIG. 5A depicts data related to the quantification of RNA export by RT-qPCR revealing that the rate of RNA export depends on copy number of the packaging signal, which is a tandem array of MS2 aptamers. Cells were transfected with RNA export system components, including reporters with varying numbers of MS2 aptamers within a tandem repeat array in the 3′ UTR, supernatant was collected, and exported RNA was measured using RT-qPCR, as shown in FIG. 1D. Results show that the rate of RNA export increases with the copy number of MS2 aptamers, and saturates at 8 copies. Dashed line indicates limit of detection, as determined by signal in control sample lacking DNA in transfection. FIG. 5B depicts data related to the quantification of RNA export by RT-qPCR reveals that RNA exporters can package their own RNA. An RNA export system was created that packages its own transcript by incorporating the packaging signal, consisting of a tandem array of eight MS2 aptamers, into the 3′ UTR of the RNA exporter transcript itself. Cells were transfected with this single RNA export system component, which serves as its own reporter, collected supernatant, and exported RNA was measured using RT-qPCR targeting GFP. Results indicate that the RNA exporter Gag-MCP efficiently exports its own transcript, if its transcript contains the packaging signal. Dashed line indicates limit of detection, as determined by signal in control sample lacking DNA in transfection. FIG. 5C depicts data related to negative stain transmission electron microscopy showing the lack of enveloped particles in supernatant collected from HEK293T cells transfected with plasmids encoding the reporter, but not the exporter. Scale bar, 100 nm. FIG. 5D depicts data related to the physical size of RNA export particles, as determined by dynamic light scattering. FIG. 5E depicts data related to the quantification of RNA export by RT-qPCR from mouse embryonic stem cells (mESCs). Species-chimeric viral RNA exporters were designed that are adapted to mouse by replacing the matrix domain (MA) from HIV Gag with MA domain from MMLV, then fusing the RNA binding domain MCP, designated Gag_MHIV_MA-MCP (top). mESCs were transfected with this chimeric RNA exporter and other components of the RNA export system and quantified RNA export (bottom). This revealed that this engineered viral species chimera can export RNA in mESCs, indicating that RNA exporters can be adapted to a broad range of cell types and species.

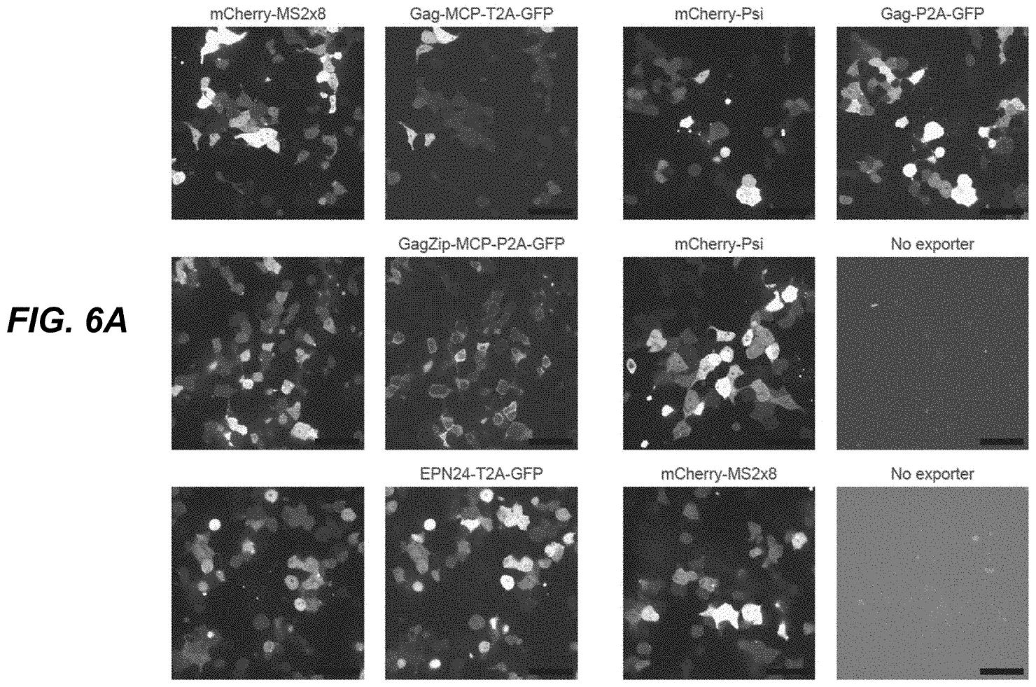

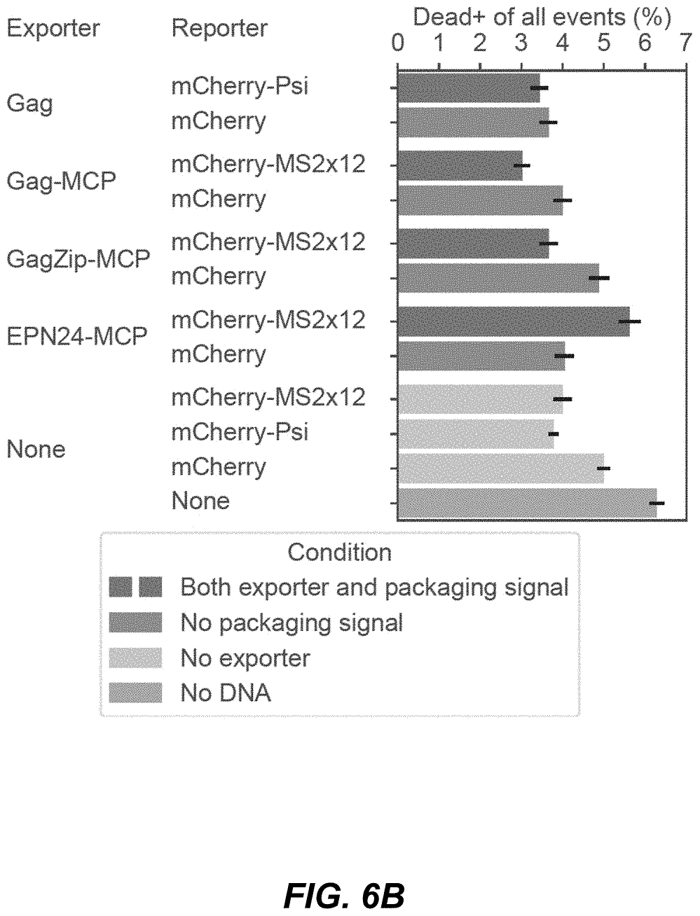

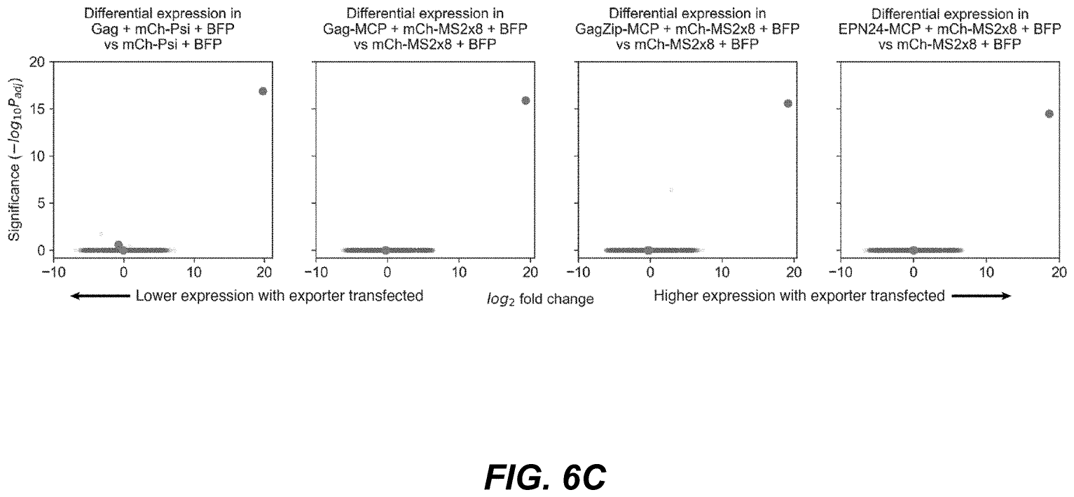

FIGS. 6A-6C depict data showing RNA exporters are non-toxic and do not perturb cellular morphology and transcriptome. FIG. 6A depicts non-limiting exemplary images of HEK293T cells transfected with RNA exporters and reporters. Scale bar, 50 um. Images reveal no apparent differences in cellular morphology between cells expressing RNA exporters and those that are not. FIG. 6B depicts data related to the quantification of toxicity of RNA exporter expression in HEK293T cells using flow cytometry with dead stain (ethidium homodimer-1). Results show that RNA exporter expression is non-toxic. Error bars show 95% confidence interval based on binomial sampling. FIG. 6C depicts data related to differential expression analysis of cellular transcriptomes of cells transfected with and without exporters. Each dot is a gene. Transgenes are shown in color. Results indicate that no genes are significantly differentially expressed in association with exporter expression, indicating that exporters do not detectably perturb cellular transcriptomes.

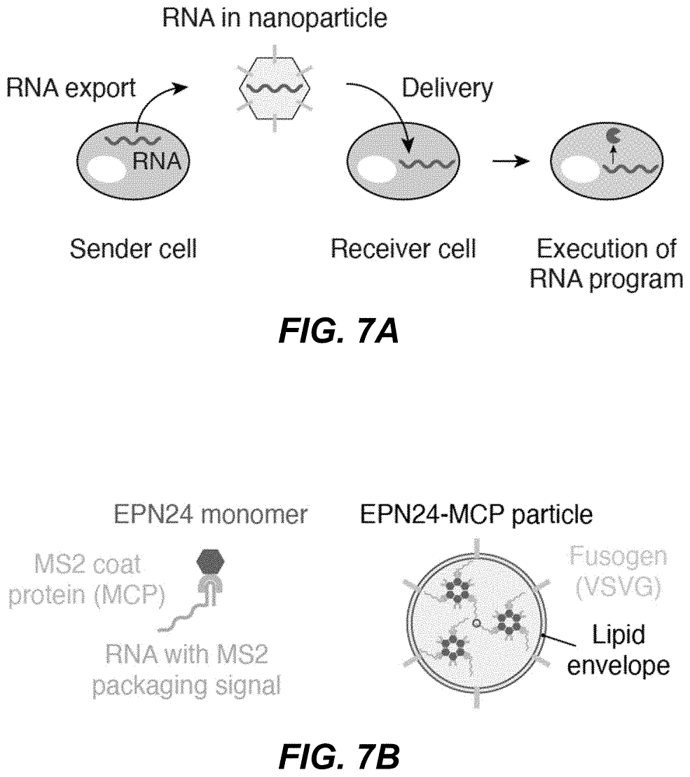

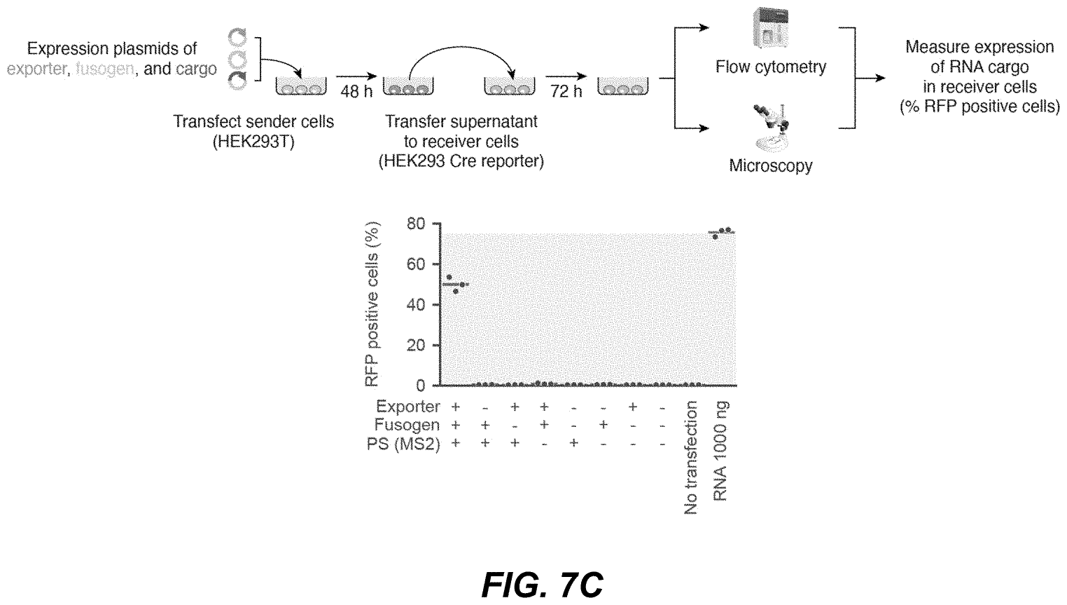

FIGS. 7A-7C depict non-limiting exemplary schematics and data related to RNA export enabling cell-to-cell delivery of therapeutic gene circuits. FIG. 7A depicts a non-limiting exemplary schematic of in situ control of living cells by cell-to-cell delivery of RNA. A sender cell exports RNA within genetically encoded nanoparticle, which delivers the RNA to a receiver cell. RNA program is executed in receiver cell, enabling control of the receiver cell's state. FIG. 7B depicts a non-limiting exemplary design of synthetic RNA delivery system. Exporter comprises enveloped protein nanocage domain EPN24 fused with RNA binding domain MS2 coat protein (MCP). Exporter self-assembles to form nanocages, which package RNA tagged with MS2 packaging signal via interaction between MCP and MS2 aptamer. Exporter particles are composed of multiple nanocages secreted from the cell within an enveloped vesicle. Delivery system also includes a fusogen, such as vesicular stomatitis virus G (VSVG) protein, which enables the nanoparticle to fuse with and deliver its RNA cargo into a receiver cell. FIG. 7C depicts a demonstration of cell-to-cell delivery of functional mRNA. Top, schematic of experiment. HEK293T sender cells were transfected with expression plasmids of exporter (EPN24-MCP), fusogen (VSVG), and cargo (Cre), supernatant was collected after 48 hours, and the supernatant was passed through 0.45 um filter. Then the supernatant was transferred to HEK293 receiver cells stably transformed with a Cre reporter locus, which activate red fluorescent protein (RFP) after Cre-mediated recombination. Cells were analyzed by flow cytometry and microscopy after 72 hours to determine activity of the RNA circuit cargo. Bottom, quantification of Cre mRNA delivery to receiver cells using flow cytometry. Cre induces expression of RFP in receiver cells. Results indicate that RNA is delivered from sender to receiver cells in an exporter-, fusogen-, and packaging signal-dependent manner. Delivery is efficient, as evidenced by Cre activity comparable to transfection of 1000 ng of Cre mRNA into receiver cells.

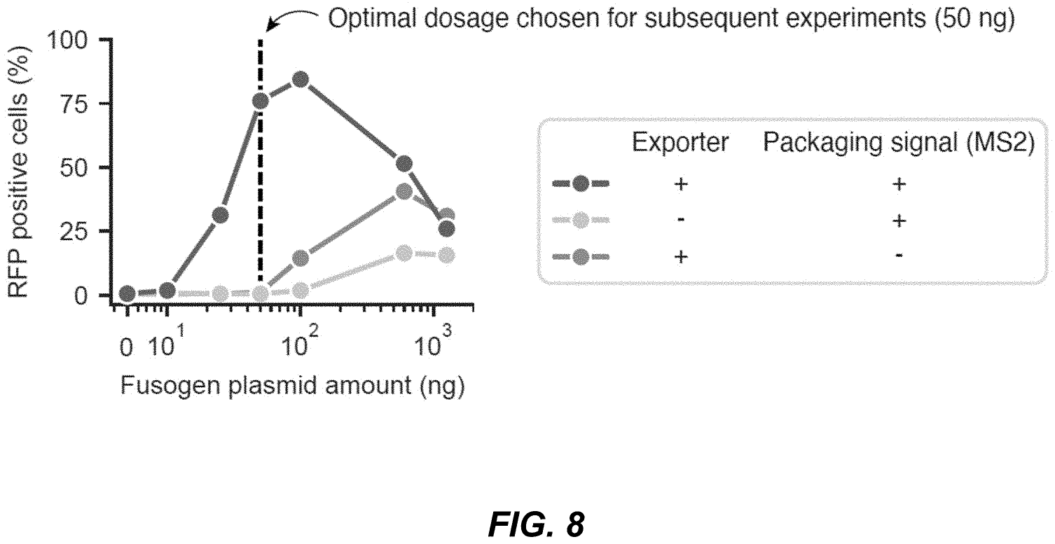

FIG. 8 depicts data related to characterization and optimization of cell-to-cell RNA delivery system. Titration of fusogen reveals optimal dosage for exporter-dependent RNA delivery. The RNA delivery assay shown in FIG. 7C was performed with transfection of varying amounts of fusogen plasmid, and determined efficiency of Cre mRNA delivery using flow cytometry. Results indicate that 50 ng of fusogen plasmid enables efficient RNA delivery, with undetectable delivery of cargo in the absence of exporter and packaging signal. This suggests that 50 ng of fusogen plasmid is optimal and this amount was used for subsequent experiments.

FIG. 9 depicts a non-limiting exemplary schematic of export systems provided herein. Engineered sender cell expresses RNA cargo and RNA exporter, which exports the cargo into an extracellular compartment. This compartment has a fusogen, enabling the compartment to fuse with and thereby deliver the RNA cargo to a receiver cell. Cargo is expressed in the receiver cell.

FIGS. 10A-10B depict non-limiting exemplary schematics and data related to demonstration of cell-to-cell transfer of RNA circuits. FIG. 10A depicts a non-limiting exemplary schematic of one exemplary system provided herein. Engineered HEK293T sender cell expresses RNA cargo (mCherry with MS2 stem-loops in 3′ untranslated region) and RNA exporter (GagZip-MCP fusion protein, consisting of modified HIV Gag protein fused to RNA binding protein MS2 coat protein). RNA exporter binds to and induces export of RNA cargo into extracellular compartment. Sender cell further expresses fusogen (e.g. VSVG), which renders the extracellular compartment capable of fusing with the receiver cell. Upon fusion, RNA cargo is delivered to the receiver cell and expressed, producing mCherry fluorescent protein. FIG. 10B depicts data related to flow cytometry revealing expression of RNA circuits transferred from sender to receiver cells. Sender cells were transfected with DNA plasmids encoding exporter (GagZip-MCP), fusogen (VSVG), and cargo (mCherry-MS2). After 48 hours, supernatant was collected from sender cells, passed through 0.45 μm filter, and transferred to receiver HEK293T cells. Expression of mCherry fluorescent protein was measured in receiver cells after 9 days. In control experiments, fusogen was omitted (No VSV-G), exporter and fusogen were omitted (mCh-MS2 alone), DNA was omitted (polybrene alone), or DNA and polybrene were omitted (no DNA, no polybrene). Results indicate that all of the system components are required for delivery and expression of RNA in receiver cells.

FIGS. 11A-11B depict data demonstrating the prevention of cargo expression in sender cells. Engineered HEK293T sender cells that constitutively express TEV Protease (TEVP) were transfected with DNA plasmid encoding RNA exporter (Gag-MCP), fusogen (VSVG), and RNA cargo either having (FIG. 11A) or lacking (FIG. 11B) a protease-regulatable N-terminal degron (N-Deg-mCherry-MS2×12 and mCherry-MS2×12, respectively). Expression of cargo was assessed by fluorescence microscopy after 48 hours. Results show that this system substantially reduces expression of the mCherry fluorescent protein reporter encoded by the RNA cargo within these sender cells.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A nucleic acid composition, comprising:
one or more first polynucleotide(s) encoding an RNA exporter protein, one or more second polynucleotide(s) each encoding one or more cargo RNA molecule(s), and one or more third polynucleotide(s) encoding a fusogen;
wherein the RNA exporter protein comprises:
an RNA-binding domain,
a membrane-binding domain, and
an interaction domain capable of nucleating self-assembly, and
wherein a plurality of RNA exporter proteins are capable of self-assembling into lipid-enveloped nanoparticles (LNs) secreted from a sender cell in which the RNA exporter proteins are expressed, thereby generating a population of LNs comprising the fusogen and exported cargo RNA molecule(s).

2. The nucleic acid composition of claim 1, wherein the population of LNs are capable of fusing with receiver cells, thereby delivering the cargo RNA molecule(s) to said receiver cells.

3. The nucleic acid composition of claim 1, wherein the fusogen is capable of mediating the fusion of the lipid envelope of the LN and a lipid bilayer of a receiver cell, and wherein the fusogen comprises or is derived from a SNARE protein, dynamin, an FF protein, a FAST protein, a viral fusogenic glycoprotein, or any combination thereof.

4. The nucleic acid composition of claim 1, wherein the one or more cargo RNA molecule(s) comprise packing signal(s) and/or wherein the cargo RNA molecule(s) are mRNA.

5. The nucleic acid composition of claim 4, wherein the RNA binding domain is capable of binding the packing signal(s), and wherein the cargo RNA molecule(s) is specifically packaged into the LNs via interaction of the packing signal(s) with the RNA-binding domain of the RNA exporter protein.

6. The nucleic acid composition of claim 4, wherein the abundance of cargo RNA molecule(s) exported to the exterior of a sender cell is at least about 2-fold higher as compared to a sender cell wherein (i) the packing signal(s) are absent from the cargo RNA molecule(s) and/or (ii) the RNA exporter protein does not comprise an RNA binding domain.

7. The nucleic acid composition of claim 4, wherein the packing signal(s) comprise an array of at least about 2, 3, 4, 5, 6, 7, 8, 9, 10, 11, 12, 13, or 14, tandem repeats of an aptamer.

8. The nucleic acid composition of claim 4, wherein the RNA binding domain comprises or is derived from an RNA binding protein, and wherein:
the packing signal(s) comprise a Ku binding hairpin and the RNA binding protein is Ku;
the packing signal(s) comprise a telomerase Sm7 binding motif and the RNA binding protein is Sm7;
the packing signal(s) comprise an MS2 phage operator stem-loop and the RNA binding protein is MS2 Coat Protein (MCP),
the packing signal(s) comprise a PP7 phage operator stem-loop and the RNA binding protein is PP7 Coat Protein (PCP);
the packing signal(s) comprise an SfMu phage Com stem-loop and the RNA binding protein is Com RNA binding protein;
the packing signal(s) comprise a PUF binding site (PBS) and the RNA binding protein is Pumilio/fem-3 mRNA binding factor (PUF); and/or
the packing signal(s) comprise an MMLV packing signal (Psi) and the RNA binding protein is MMLV.

9. The nucleic acid composition of claim 1, wherein the RNA exporter protein comprises at least a portion of a viral capsid protein.

10. The nucleic acid composition of claim 1, wherein the RNA exporter protein comprises an amino acid sequence that is at least 80%, 85%, 90%, 95%, 98%, 99%, or 100% identical to any one of SEQ ID NOS: 1-24.

11. The nucleic acid composition of claim 1, wherein the expression of the RNA exporter protein in the sender cell(s) does not alter the endogenous transcriptome, morphology, and/or physiology of said sender cell(s).

12. The nucleic acid composition of claim 2, wherein one or more cargo RNA molecule(s) encode one or more payload protein(s), and wherein said payload proteins are capable of being translated upon delivery to the receiver cell(s).

13. The nucleic acid composition of claim 12, wherein the cargo RNA molecule(s) and/or payload protein(s) encoded by said cargo RNA molecule(s) constitute two or more components of a receiver circuit.

14. The nucleic acid composition of claim 13, wherein the receiver circuit:
is capable of modulating cell states, cell types, and/or cell behaviors;
is configured to selectively activate cell death and/or immune recruitment to tumor cells;
is configured to detect the intracellular state of the receiver cell and classifying it as tumor or normal based on the levels or activities of relevant molecules or pathways;
is configured to reprogram a receiver cell type and/or cell state via expression of transcription factors and/or epigenetic modifiers;
is configured to reprogram a receiver cell type and/or cell state via expression of transcription factors and/or epigenetic modifiers;
is capable of directly or indirectly inducing cell death in the presence of the aberrant signaling of one or more signal transducer(s);
is capable of detecting aberrant signaling, an activity of a signal transducer, an activity of a signal transducer activator and/or an activity of a signal transducer repressor, wherein the detecting comprises detecting a modification selected from the group comprising phosphorylation, dephosphorylation, acetylation, methylation, acylation, glycosylation, glycosylphosphatidylinositol (GPI) anchoring, sulfation, disulfide bond formation, deamidation, ubiquitination, sumoylation, nitration of tyrosine, hydrolysis of ATP or GTP, binding of ATP or GTP, cleavage, or any combination thereof; and/or
is capable of reprogramming a receiver cell from a first cell type and/or first cell state to a second cell type and/or second cell state.

15. The nucleic acid composition of claim 12, wherein a payload protein is capable of modulating the expression, concentration, localization, stability, and/or activity of the one or more endogenous proteins of a receiver cell.

16. The nucleic acid composition of claim 12, wherein a payload protein comprises:
fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof;
nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof;
a therapeutic protein configured to prevent or treat a disease or disorder of a subject, wherein said subject suffers from a deficiency of said therapeutic protein;
a cellular reprogramming factor capable of differentiating a given cell into a desired differentiated state selected from the group comprising nerve growth factor (NGF), fibroblast growth factor (FGF), interleukin-6 (IL-6), bone morphogenic protein (BMP), neurogenin3 (Ngn3), pancreatic and duodenal homeobox 1 (Pdx1), Mafa, or any combination thereof;
an agonistic or antagonistic antibody or antigen-binding fragment thereof specific to a checkpoint inhibitor or checkpoint stimulator molecule selected from the group comprising PD1, PD-L1, PD-L2, CD27, CD28, CD40, CD137, OX40, GITR, ICOS, A2AR, B7-H3, B7-H4, BTLA, CTLA4, IDO, KIR, LAG3, PD-1, and/or TIM-3;
a secretion tag, wherein the secretion tag is selected from the group comprising AbnA, AmyE, AprE, BglC, BglS, Bpr, Csn, Epr, Ggt, GlpQ, HtrA, LipA, LytD, MntA, Mpr, NprE, OppA, PbpA, PbpX, Pel, PelB, PenP, PhoA, PhoB, PhoD, PstS, TasA, Vpr, WapA, WprA, XynA, XynD, YbdN, Ybxl, YcdH, YclQ, YdhF, YdhT, YfkN, YflE, YfmC, Yfnl, YhcR, YlqB, YncM, YnfF, YoaW, YocH, YolA, YqiX, Yqxl, YrpD, YrpE, YuaB, Yurl, YvcE, YvgO, YvpA, YwaD, YweA, YwoF, YwtD, YwtF, YxaLk, YxiA, and YxkC;
a constitutive signal peptide for protein degradation;
a nuclear localization signal (NLS) or a nuclear export signal (NES);
a dosage indicator protein, wherein the dosage indicator protein is detectable, and wherein the dosage indicator protein comprises green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof;
a cellular reprogramming factor capable of converting an at least partially differentiated cell to a less differentiated cell selected from the group comprising Oct-3, Oct-4, Sox2, c-Myc, Klf4, Nanog, Lin28, ASCL1, MYT1L, TBX3b, SV40 large T, hTERT, miR-291, miR-294, miR-295, or any combinations thereof;
a programmable nuclease selected from the group comprising: SpCas9 or a derivative thereof; VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; Cas13-adenosine deaminase fusions;
a CRE recombinase, GCaMP, a cell therapy component, a knock-down gene therapy component, a cell-surface exposed epitope, or any combination thereof;
a bispecific T cell engager (BiTE);
a cytokine selected from the group consisting of interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, interleukin-1 (IL-1), IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, granulocyte macrophage colony stimulating factor (GM-CSF), M-CSF, SCF, TSLP, oncostatin M, leukemia-inhibitory factor (LIF), CNTF, Cardiotropin-1, NNT-1/BSF-3, growth hormone, Prolactin, Erythropoietin, Thrombopoietin, Leptin, G-CSF, or receptor or ligand thereof;
a member of the TGF-β/BMP family selected from the group consisting of TGF-β1, TGF-β2, TGF-β3, BMP-2, BMP-3a, BMP-3b, BMP-4, BMP-5, BMP-6, BMP-7, BMP-8a, BMP-8b, BMP-9, BMP-10, BMP-11, BMP-15, BMP-16, endometrial bleeding associated factor (EBAF), growth differentiation factor-1 (GDF-1), GDF-2, GDF-3, GDF-5, GDF-6, GDF-7, GDF-8, GDF-9, GDF-12, GDF-14, mullerian inhibiting substance (MIS), activin-1, activin-2, activin-3, activin-4, and activin-5;
a member of the TNF family of cytokines selected from the group consisting of TNF-alpha, TNF-beta, LT-beta, CD40 ligand, Fas ligand, CD 27 ligand, CD 30 ligand, and 4-1 BBL;
a member of the immunoglobulin superfamily of cytokines selected from the group consisting of B7.1 (CD80) and B7.2 (B70);
an interferon selected from the group comprising interferon alpha, interferon beta, or interferon gamma;
a chemokine selected from the group comprising CCL1, CCL2, CCL3, CCR4, CCL5, CCL7, CCL8/MCP-2, CCL11, CCL13/MCP-4, HCC-1/CCL14, CTAC/CCL17, CCL19, CCL22, CCL23, CCL24, CCL26, CCL27, VEGF, PDGF, lymphotactin (XCL1), Eotaxin, FGF, EGF, IP-10, TRAIL, GCP-2/CXCL6, NAP-2/CXCL7, CXCL8, CXCL10, ITAC/CXCL11, CXCL12, CXCL13, or CXCL15;
an interleukin selected from the group comprising IL-10 IL-12, IL-1, IL-6, IL-7, IL-15, IL-2, IL-18 or IL-21;
a tumor necrosis factor (TNF) selected from the group comprising TNF-alpha, TNF-beta, TNF-gamma, CD252, CD154, CD178, CD70, CD153, or 4-1BBL;
a factor locally down-regulating the activity of endogenous immune cells;
is capable of remodeling a tumor microenvironment and/or reducing immunosuppression at a target site of a subject; and/or
a chimeric antigen receptor (CAR) or T-cell receptor (TCR).

17. The nucleic acid composition of claim 1, wherein the LNs contacted with RNase are capable of protecting cargo RNA molecule(s) comprised therein from RNase-mediated degradation.

18. The nucleic acid composition of claim 1, wherein:
the average diameter of the LNs of the population of LNs is about 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 300 nm, 400 nm, or 500 nm; and/or
the LNs have a minimum diameter and/or a maximum diameter of about 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, 205 nm, 210 nm, 215 nm, 220 nm, 225 nm, 230 nm, 235 nm, 240 nm, 245 nm, 250 nm, 300 nm, 400 nm, or 500 nm.

19. A population of sender cells, comprising:
one or more first polynucleotide(s) encoding an RNA exporter protein, one or more second polynucleotide(s) each encoding one or more cargo RNA molecule(s), and one or more third polynucleotide(s) encoding a fusogen;
wherein the RNA exporter protein comprises:
an RNA-binding domain,
a membrane-binding domain, and
an interaction domain capable of nucleating self-assembly, and
wherein a plurality of RNA exporter proteins are capable of self-assembling into lipid-enveloped nanoparticles (LNs) secreted from a sender cell in which the RNA exporter proteins are expressed, thereby generating a population of LNs comprising the fusogen and exported cargo RNA molecule(s).

20. A method of treating or preventing a disease or disorder in a subject in need thereof, comprising:
administering to the subject an effective amount of a nucleic acid composition or a population of sender cells, wherein the nucleic acid composition or the population of sender cells comprises:
one or more first polynucleotide(s) encoding an RNA exporter protein, one or more second polynucleotide(s) each encoding one or more cargo RNA molecule(s), and one or more third polynucleotide(s) encoding a fusogen,
wherein the RNA exporter protein comprises:
an RNA-binding domain,
a membrane-binding domain, and
an interaction domain capable of nucleating self-assembly, and
wherein a plurality of RNA exporter proteins are capable of self-assembling into lipid-enveloped nanoparticles (LNs) secreted from a sender cell in which the RNA exporter proteins are expressed, thereby generating a population of LNs comprising the fusogen and exported cargo RNA molecule(s), thereby treating or preventing the disease or disorder in the subject.


SEQUENCE LISTING (24 sequences)

SEQ ID NO: 1 - protein, 282 aa, synthetic construct
MGARASGSKSGSGSDSGSKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLQSRPEPTAPPEESFRSGVETTTPPQKQEPIDKELYPLTSLRSLFGNDPSSQ

SEQ ID NO: 2 - protein, 411 aa, synthetic construct
MGARASGSKSGSGSDSGSKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLQSRPEPTAPPEESFRSGVETTTPPQKQEPIDKELYPLTSLRSLFGNDPSSQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 3 - protein, 411 aa, synthetic construct
MGARASGSKSGSGSDSGSKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYEQKLISEEDLQSRPEPTAPPEESFRSGVETTTPPQKQEPIDKELYPLTSLRSLFGNDPSSQ

SEQ ID NO: 4 - protein, 412 aa, synthetic construct
MGARASGSKSGSGSDSGSKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYLQSRPEPTAPPEESFRSGVETTTPPQKQEPIDKELYPLTSLRSLFGNDPSSQ

SEQ ID NO: 5 - protein, 278 aa, synthetic construct
MGCIKSKRKDNLNLQSRPEPTAPPEESFRSGVETTTPPQKQEPIDKELYPLTSLRSLFGNDPSSQKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDL

SEQ ID NO: 6 - protein, 407 aa, synthetic construct
MGCIKSKRKDNLNLQSRPEPTAPPEESFRSGVETTTPPQKQEPIDKELYPLTSLRSLFGNDPSSQKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 7 - protein, 407 aa, synthetic construct
MGCIKSKRKDNLNLQSRPEPTAPPEESFRSGVETTTPPQKQEPIDKELYPLTSLRSLFGNDPSSQKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYEQKLISEEDL

SEQ ID NO: 8 - protein, 407 aa, synthetic construct
MGCIKSKRKDNLNLQSRPEPTAPPEESFRSGVETTTPPQKQEPIDKELYPLTSLRSLFGNDPSSQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDL

SEQ ID NO: 9 - protein, 396 aa, synthetic construct
MHGLQDDPDLQALLKGSQLLKVKSSSWRRERFYKLQEDCKTIWQESRKVMRSPESQLFSIEDIQEVRMGHRTEGLEKFARDIPEDRCFSIVFKDQRNTLDLIAPSPADAQHWVQGLRKIIHHSGSMDQRQKLQSRPEPTAPPEESFRSGVETTTPPQKQEPIDKELYPLTSLRSLFGNDPSSQKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDL

SEQ ID NO: 10 - protein, 525 aa, synthetic construct
MHGLQDDPDLQALLKGSQLLKVKSSSWRRERFYKLQEDCKTIWQESRKVMRSPESQLFSIEDIQEVRMGHRTEGLEKFARDIPEDRCFSIVFKDQRNTLDLIAPSPADAQHWVQGLRKIIHHSGSMDQRQKLQSRPEPTAPPEESFRSGVETTTPPQKQEPIDKELYPLTSLRSLFGNDPSSQKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDLASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 11 - protein, 525 aa, synthetic construct
MHGLQDDPDLQALLKGSQLLKVKSSSWRRERFYKLQEDCKTIWQESRKVMRSPESQLFSIEDIQEVRMGHRTEGLEKFARDIPEDRCFSIVFKDQRNTLDLIAPSPADAQHWVQGLRKIIHHSGSMDQRQKLQSRPEPTAPPEESFRSGVETTTPPQKQEPIDKELYPLTSLRSLFGNDPSSQKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYEQKLISEEDL

SEQ ID NO: 12 - protein, 525 aa, synthetic construct
MHGLQDDPDLQALLKGSQLLKVKSSSWRRERFYKLQEDCKTIWQESRKVMRSPESQLFSIEDIQEVRMGHRTEGLEKFARDIPEDRCFSIVFKDQRNTLDLIAPSPADAQHWVQGLRKIIHHSGSMDQRQKLQSRPEPTAPPEESFRSGVETTTPPQKQEPIDKELYPLTSLRSLFGNDPSSQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYKIEELFKKHKIVAVLRANSVEEAKKKALAVFLGGVHLIEITFTVPDADTVIKELSFLKEMGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPVEVAEKAKAFVEKIRGCTEQKLISEEDL

SEQ ID NO: 13 - protein, 500 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 14 - protein, 534 aa, synthetic construct
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLL

SEQ ID NO: 15 - protein, 628 aa, synthetic construct
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 16 - protein, 629 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 17 - protein, 629 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTEASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYRQANFLGKIWPSYKGRPGNFLQSRPEPTAPPEESFRSGVETTTPPQKQEPIDKELYPLTSLRSLFGNDPSSQ

SEQ ID NO: 18 - protein, 616 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTHNPPIPVGEIYKRWIILGLNKIVRMYSPTSILDIRQGPKEPFRDYVDRFYKTLRAEQASQEVKNWMTETLLVQNANPDCKTILKALGPGATLEEMMTACQGVGGPGHKARVLAEAMSQVTNPATIMIQKGNFRNQRKTVKCFNCGKEGHIAKNCRAPRKKGVASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYTERQANFLGKIWPSHKGRPGNFLQSRPEPTAPPEESFRFGEETTTPSQKQEPIDKELYPLASLRSLFGSDPSSQ

SEQ ID NO: 19 - protein, 608 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMLQRMKQLEDKVEELLSKNYHLENEVARLKKLVGEFLGKIWPSYKGRPGNFLQSRPEPTAPPEESFRSGVETTTPPQKQEPIDKELYPLTSLRSLFGNDPSSQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 20 - protein, 629 aa, synthetic construct; identical to SEQ ID NO: 16

SEQ ID NO: 21 - protein, 608 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMLQRMKQLEDKVEELLSKNYHLENEVARLKKLVGEASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYFLGKIWPSYKGRPGNFLQSRPEPTAPPEESFRSGVETTTPPQKQEPIDKELYPLTSLRSLFGNDPSSQ

SEQ ID NO: 22 - protein, 540 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMLQRMKQLEDKVEELLSKNYHLENEVARLKKLVGEASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 23 - protein, 322 aa, synthetic construct
MGARASVSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMLQRMKQLEDKVEELLSKNYHLENEVARLKKLVGELQSRPEPTAPPEESFRSGVETTTPPQKQEPIDKELYPLTSLRSLFGNDPSSQASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY

SEQ ID NO: 24 - protein, 270 aa, synthetic construct
MGARASVSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMLQRMKQLEDKVEELLSKNYHLENEVARLKKLVGEASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIY